(19) United States
(12) Patent Application Publication
(10) Pub. No.: US 2011/0059091 A1
(43) Pub. Date: Mar. 10, 2011

(54) INHIBITORS OF ONCOGENIC ISOFORMS AND USES THEREOF

(76) Inventors: Xiao-Jia Chang, Lincoln, MA (US); Ullrich S. Schwertschlag, Danvers, MA (US)

(21) Appl. No.: 12/866,013

(22) PCT Filed: Feb. 4, 2009
(86) PCT No.: PCT/US2009/033031

§ 371 Date: Nov. 22, 2010

Related U.S. Application Data
(60) Provisional application No. 61/025,947, filed on Feb. 4, 2008.

(51) Int. Cl. A61K 39/395; C07K 16/40; C07K 16/28; C07K 16/22; C07K 2/00; C07H 21/00; C07K 16/46; A61K 31/7088; A61K 38/02; C12N 5/071; C12P 21/02; G01N 33/53; C07K 14/47; C07K 7/06; C07K 5/103; C07K 7/08; C12N 5/09; C12Q 1/68; G01N 33/68; A61P 35/00

(52) U.S. Cl. 424/138.1; 530/387.7; 530/300; 536/24.5; 530/387.3; 530/391.3; 530/391.7; 514/44 R; 514/1.1; 536/23.4; 536/23.53; 536/23.1; 435/325; 435/69.2; 435/69.6; 435/69.7; 435/69.1; 436/501; 530/324; 530/327; 530/328; 530/330; 530/326; 435/375; 435/6; 435/7.1

(57) ABSTRACT

Isoform-binding molecules that specifically bind to one or more isoforms expressed and/or associated with oncogenic phenotypes in a hyperproliferative cell (e.g., a cancerous or tumor cell) are disclosed. The isoform-binding molecules can be used to treat, prevent and/or diagnose cancerous conditions and/or disorders. Methods of using the isoform-binding molecules to selectively detect oncogenic isoforms, to reduce the activity and/or induce the killing of a hyperproliferative cell expressing an oncogenic isoform in vitro, ex vivo or in vivo are also disclosed. Diagnostic and/or screening methods and kits for evaluating the function or expression of an oncogenic isoform are also disclosed.

### CROSS REFERENCE TO RELATED APPLICATIONS

This application claims priority to U.S. Ser. No. 61/025,947 filed on Feb. 4, 2008. The contents of the aforementioned application are hereby incorporated by reference in their entirety.

### GOVERNMENT SUPPORT

The work described herein was carried out, at least in part, using funds from the United States government under contract number 1R43CA137929-01, from the National Institutes of Health (NIH). The U.S. government may therefore have certain rights in the invention.

### BACKGROUND

In spite of numerous advances in medical research, cancer remains a leading cause of death in the United States. Traditional modes of clinical care, such as surgical resection, radiotherapy and chemotherapy, have a significant failure rate, especially for solid tumors. Failure occurs either because the initial tumor is unresponsive, or because of recurrence due to re-growth at the original site and/or metastases. The etiology, diagnosis and ablation of cancer remain a central focus for medical research and development.

Since the probability of complete remission of cancer is, in most cases, greatly enhanced by early diagnosis, it is desirable for physicians to be able to identify cancerous tumors as early as possible. Identification of cancerous cells based on changes in gene expression is desirable because changes in gene expression are likely to occur prior to the histological changes that distinguish malignant cells from normal cells. Using biomarkers that identify such changes in gene expression, one can identify cancerous or pre-cancerous cells when changes in gene expression are apparent, and thereby effectively target individuals who would most likely benefit from adjuvant therapy. However, the development of methods and compositions that permit early, rapid, and accurate detection of many forms of cancers continues to challenge the medical community. Thus, a significant problem in the treatment of cancer remains detection and prognosis to enable appropriate therapeutic treatment and ablation of cancer.

For example, prostate cancer (CaP) is one of the most common malignancies in men, with an increasing incidence. In 2007, approximately 218,900 men were diagnosed and approximately 27,050 men died of the disease in the U.S. alone. Despite important progress in the early diagnosis of prostate malignancies through the measurement of PSA levels, about 10% of newly diagnosed patients have some evidence of locally advanced CaP and 5% already have distant metastasis at the time of diagnoses (Draisma et al., (2003) J. Natl. Cancer Inst. 95:868-878; Thompson et al., (2003) N. Engl. J. Med. 349, 215-224; Makinen et al., (2003) Clin. Cancer Res. 9, 2435-2439). Curative treatments for locally advanced CaP are available (Bolla et al., (2002) Lancet 360, 103-106; Messing et al., (1999) N. Engl. J. Med. 341, 1781-1788; D'Amico et al., (2004) J. Am. Med. Assoc. 292, 821-827). In contrast, patients with evidence of distant metastases have a very poor prognosis and limited curative treatment exists (Cheville et al., (2002) Cancer 95, 1028-1036). Tumor metastasis is the main cause for mortality associated with prostate cancer. Hormone-refractory prostate cancer (HRPC) is an example of an invasive type of prostate cancer.

Limited treatment modalities currently exist for prostate cancer once it has metastasized. For example, systemic therapy is limited to various forms of androgen deprivation. While most patients will demonstrate initial clinical improvement, virtually inevitably, androgen-independent cells develop. Endocrine therapy is thus palliative, not curative. In a study of 1387 patients with metastatic disease detectable by imaging (e.g., bone or CT scan), the median time to objective disease progression (excluding biochemical/PSA progression) after initiation of hormonal therapy (i.e., development of androgen-independence) was 16-48 months (Eisenberger M. A., et al. (1998) NEJM 339:1036-42). Median overall survival in these patients was 28-52 months from the onset of hormonal treatment (Eisenberger M. A., et al. (1998) supra.). Subsequent to developing androgen-independence, there is no effective standard therapy and the median duration of survival is 9-12 months (Vollmer, R. T., et al. (1999) Clin Can Res 5: 831-7; Hudes G., et al., (1997) Proc Am Soc Clin Oncol 16:316a (abstract); Pienta K. J., et al. (1994) J Clin Oncol 12(10):2005-12; Pienta K. J., et al. (1997) Urology 50:401-7; Tannock I. F., et al., (1996) J Clin Oncol 14:1756-65; Kantoff P. W., et al., (1996) J. Clin. Oncol. 15 (Suppl):25:110-25). Cytotoxic chemotherapy is poorly tolerated in this age group and generally considered ineffective and/or impractical. In addition, prostate cancer is relatively resistant to cytotoxic agents. Thus, chemotherapeutic regimen has not demonstrated a significant survival benefit in this patient group.

In 2004, two landmark trials using docetaxel-based chemotherapy, TAX 327 and SWOG 99-16, showed a survival benefit for the first time in metastatic, hormone-refractory prostate cancer (Tannock et al., (2004) N. Engl. J. Med. 351, 1502-1512; Petrylak et al. (2004) N. Engl. J. Med. 351, 1513-1520). However, these chemotherapies have multiple toxicities and only prolonged patients' lives for approximately 2.5 months. Current research suggests that several distinct mechanisms of androgen-refractory disease may converge in patients with disease progression on androgen deprivation therapy. These findings have identified several potential targets for therapeutic intervention. Ongoing studies for investigational new drugs include anti-angiogenic therapies, signal transduction inhibitors, immunomodulatory agents, and nuclear receptor targets (reviewed in Mendiratta et al., (2007) Rev Urol. 9(Suppl 1): S9-S19).

In view of the shortcomings of existing therapies and diagnostics, the need still exists for improved targeted modalities for preventing, treating and/or diagnosing cancers, such as prostate cancer.

### SUMMARY

The present invention features, at least in part, isoform-specific inhibitors that inhibit or reduce one or more isoform-associated activities, wherein the isoform-specific inhibitors include but are not limited to, binding molecules (also referred to herein as “isoform-binding molecules”) that specifically interact with, e.g., bind to, one or more isoforms (e.g., isoform polypeptides or nucleic acids encoding the same) that arise from, e.g., one or more of: alternative splicing, frameshifting, translational and/or post-translational events, thereby resulting in different transcription or translation products. In one embodiment, the isoform-specific inhibitors specifically bind to, and/or inhibit the activity of one or more isoforms expressed and/or associated with oncogenic or malignant phenotypes (referred to herein as “oncogenic isoforms”). For example, the isoform-specific inhibitor can be an oncogenic isoform-binding molecule, e.g., an antibody molecule or a nucleic acid inhibitor that specifically interacts with, e.g., binds to, one or more oncogenic isoforms (e.g., oncogenic isoform polypeptides or nucleic acids encoding the same). In another embodiment, the isoform-specific inhibitor is a soluble receptor polypeptide or a fusion form thereof, or a peptide or a functional variant thereof that reduces or inhibits one or more isoform- (e.g., oncogenic isoform-) associated activities. In embodiments, the soluble receptor or fusion reduce or inhibit (e.g., competitively inhibit) an interaction of the isoform (e.g., the oncogenic isoform) polypeptide and its cognate ligand or receptor.

The oncogenic isoforms can arise from, e.g., alternative splicing, frameshifting, translational and/or post-translational events, of various proto-oncogene expression products in a cell, e.g., a hyperproliferative cell (e.g., a cancerous or tumor cell). The isoform-binding molecules described herein specifically bind to such oncogenic isoforms, and do not substantially bind to the proto-oncogene from which the isoform is derived. In certain embodiments, the isoform-binding molecule specifically interacts with, e.g., binds to, an oncogenic isoform of: fibroblast growth factor receptor 2 (FGFR2) (e.g., an oncogenic FGFR2 isoform IIIc); fibroblast growth factor receptor 1 (FGFR1) (e.g., an oncogenic FGFR1L); RON receptor tyrosine kinase (c-met-related tyrosine kinase) (e.g., an oncogenic RON receptor tyrosine kinase comprising a deletion of exons 5 and 6); KIT receptor tyrosine kinase (e.g., an oncogenic KIT receptor tyrosine kinase comprising a deletion in exon 11); platelet-derived growth factor (PDGF) (e.g., an oncogenic PDGF isoform having a deletion in exon 6); or PDGF-receptor alpha (e.g., an oncogenic PDGF-receptor alpha comprising a deletion of exons 7 and 8). Thus, the binding molecules that specifically bind to an oncogenic isoform provided herein can be used to identify cancerous or tumor cells associated with expression of the oncogenic isoform.

Accordingly, the present invention provides, in part, isoform-specific inhibitors (e.g., antibody molecules, soluble receptor polypeptides and fusion forms thereof, peptides and functional variants thereof, and nucleic acid inhibitors), pharmaceutical compositions thereof, as well as nucleic acids, recombinant expression vectors and host cells for making such isoform-binding molecules. In certain embodiments, the isoform-specific inhibitors selectively bind to and/or reduce, inhibit or otherwise block an interaction of an oncogenic isoform with a ligand or co-receptor, thereby reducing or inhibiting oncogenic activity. In some embodiments, the isoform-specific inhibitors compete for binding of a cognate ligand (e.g., FGF8b) to the isoform (e.g., FGFR2-IIIc). In other embodiments, the isoform-specific inhibitors act as dominant negative competitors, e.g., a dominant negative competitor that binds to the isoform but does not produce intracellular signal. In other embodiments, the isoform-binding molecules may selectively target a cytotoxic or cytostatic agent to a hyperproliferative cell, e.g., a cancer or tumor cell. The isoform-specific inhibitors disclosed herein can be used to treat, prevent and/or diagnose cancerous or malignant conditions and/or disorders, such as cancers or tumors (primary, recurring or metastasizing), including but not limited to, prostatic, bladder, breast, pancreatic, ovarian, brain (glioblastoma) and gastrointestinal cancers. Methods of using the isoform-binding molecules of the invention to detect oncogenic isoforms, to reduce the activity and/or or kill a hyperproliferative cell expressing an oncogenic isoform in vitro, ex vivo or in vivo are also encompassed by the invention. Diagnostic and/or screening methods and kits for evaluating the function or expression of an oncogenic isoform are also disclosed.

Accordingly, in one aspect, the invention features an isoform-specific inhibitor (e.g., an antibody molecule, a soluble receptor polypeptide and a fusion form thereof, a peptide and a functional variant thereof, and a nucleic acid inhibitor (e.g., an antisense nucleic molecule, an RNAi molecule or an aptamer molecule)), which interacts with, or more preferably specifically binds to, one or more isoform polypeptides or fragments thereof, or nucleic acids encoding one or more isoform polypeptides or fragments thereof. Typical isoform-binding molecules bind to one or more isoform polypeptides or fragments thereof, or nucleic acids encoding one or more isoform polypeptides or fragments thereof, with high affinity, e.g., with an affinity constant of at least about 10⁷ M⁻¹, typically about 10⁸ M⁻¹, and more typically, about 10⁹ M⁻¹ to 10¹⁰ M⁻¹ or stronger; and reduce and/or inhibit one or more activities of the isoforms, e.g., oncogenic isoforms, in a hyperproliferative (e.g., cancerous or malignant) cell and/or tissue. For example, the binding molecule may selectively and specifically reduce or inhibit an oncogenic isoform-associated activity chosen from one or more of: (i) binding of a ligand or co-receptor (e.g., FGF ligand, e.g., FGF8b, FGF2, FGF17 or FGF18 to FGFR2 isoform IIIc); (ii) receptor dimerization (e.g., FGFR2 isoform IIIc dimerization); (iii) isoform signaling, e.g., FGFR2 isoform IIIc signaling; (iv) hyperproliferative (e.g., cancerous or tumor) cell proliferation, growth and/or survival, for example, by induction of apoptosis of the hyperproliferative cell; and/or (v) angiogenesis and/or vascularization of a tumor. In certain embodiments, the inhibitor may exert its effects directly in the hyperproliferative (e.g., cancerous or malignant) cell and/or tissue (e.g., inducing cell killing or apoptosis directly). In other embodiments, the inhibitor can exert its effects by acting on proximal cells, e.g., cells in the vicinity, of the hyperproliferative (e.g., cancerous or malignant) cell and/or tissue. For example, the inhibitor may reduce the angiogenesis and/or vascularization of a tumor tissue.

In one embodiment, the isoform-binding molecule is an antibody molecule that binds to a mammalian, e.g., human, isoform polypeptide or a fragment thereof. For example, the antibody molecule binds to an isoform polypeptide or fragment expressed and/or associated with a hyperproliferative cell, e.g., a cancerous or tumor cell. For example, the antibody molecule binds specifically to an epitope, e.g., a linear or conformational epitope, located or expressed primarily on the surface of a hyperproliferative cell, e.g., a cancerous or tumor cell. In embodiments, the epitope recognized by the antibody molecule is expressed or associated with a hyperproliferative disease, e.g., a cancerous or malignant disease. For example, the epitope recognized by the antibody molecule is expressed or associated with an exon sequence predominantly expressed or associated with one or more cancerous or tumor cells or disorders; the epitope may be located at the junctional region between two exons that are predominantly joined together in one or more cancerous or tumor cells or disorders, e.g., as a result of an in-frame exon deletion or the use of an alternatively spliced exon. Exemplary isoform polypeptides or fragments recognized by isoform-binding molecules of the invention include, but are not limited to, oncogenic isoforms of FGFR2, FGFR1, RON receptor tyrosine kinase, KIT receptor tyrosine kinase, PDGF and PDGF-receptor alpha.

In one embodiment, the antibody molecule binds to an isoform, e.g., an oncogenic isoform, of FGFR2, e.g., human FGFR2. The antibody molecule can bind specifically to FGFR2 isoform IIIc or a fragment thereof, e.g., does not substantially bind to other non-oncogenic isoforms of the FGF receptors, such as other alternative splice variants of FGFR2 (e.g., FGFR2IIIb (SEQ ID NO: 21), FGFR2 isoform 4 (SEQ ID NO: 22), FGFR2 isoform 7 (SEQ ID NO: 23), FGFR2 isoform 9 (SEQ ID NO: 24), FGFR2 isoform 10 (SEQ ID NO: 25), FGFR2 isoform 11 (SEQ ID NO: 26), FGFR2 isoform 12 (SEQ ID NO: 27), FGFR2 isoform 13 (SEQ ID NO: 28), FGFR2 isoform 14 (SEQ ID NO: 29), FGFR2 isoform 15 (SEQ ID NO: 30), FGFR isoform 17 (SEQ ID NO: 31), FGFR2 isoform 18 (SEQ ID NO: 52), or FGFR2 isoform 19 (SEQ ID NO: 53)). For example, the antibody molecule binds preferentially to FGFR2 isoform IIIc or a fragment thereof, but does not substantially bind to (e.g., shows less than 10%, 8%, 5%, 4%, 3%, 2%, 1% cross-reactivity with) FGFR2 isoform IIIb, e.g., about amino acids 314 to 351 of human FGFR2 isoform IIIb (HSGINSSNAEVLALFNVTEADAGEYICKVSNYIGQANQ; SEQ ID NO: 56); about amino acids 314 to 328 of human FGFR2 isoform IIIb (HSGINSSNAEVLALF; SEQ ID NO: 57); or about amino acids 340 to 351 of human FGFR2 isoform IIIb (CKVSNYIGQANQ; SEQ ID NO: 58). In those embodiments, the antibody molecule binds specifically to at least one epitope located in the alternative spliced form of Exon III, e.g., from about amino acids 301 to 360 of FGFR2-IIIc (SEQ ID NO:2); about amino acids 314 to 324 of FGFR2-IIIc (AAGVNTTDKEI, SEQ ID NO:4); about amino acids 328 to 337 of FGFR2-IIIc (YIRNVTFEDA, SEQ ID NO:6); about amino acids 350 to 353 of FGFR2-IIIc (ISFH, SEQ ID NO:8), or an amino acid sequence encoded by a nucleotide sequence of SEQ ID NOs: 1, 3, 5 or 7; or an amino acid or nucleotide sequence substantially identical thereto.

In another embodiment, the antibody molecule binds specifically to an isoform, e.g., an oncogenic isoform, of FGFR1, e.g., human FGFR1. For example, the antibody molecule binds specifically to isoform FGFR1L having a deletion of about 105 amino acids between exons 7 and 8, corresponding to part of immunoglobulin domain II (Ig-II) and part of Ig-III of FGFR1, thus forming a junctional region between II:III. For example, the antibody molecule binds preferentially to FGFR1L or a fragment thereof, but does not substantially bind to (e.g., shows less than 10%, 8%, 5%, 4%, 3%, 2%, 1% cross-reactivity with) FGFR1 (e.g., non-oncogenic human FGFR1, e.g., FGFR1 isoform 4 (SEQ ID NO: 39), FGFR1 isoform 14 (SEQ ID NO: 40), FGFR1 isoform 16 (SEQ ID NO: 41), FGFR1 isoform 17 (SEQ ID NO: 42), FGFR1 isoform 3 (SEQ ID NO: 43), or FGFR1 isoform 18 (SEQ ID NO: 44). In those embodiments, the antibody molecule binds specifically to at least one epitope found at the junctional region between Ig-II and Ig-III of SEQ ID NO:10 or a fragment thereof, or an amino acid sequence encoded by a nucleotide sequence of SEQ ID NO:9 or a fragment thereof; or an amino acid or nucleotide sequence substantially identical thereto.

In yet other embodiments, the antibody molecule binds to an isoform, e.g., an oncogenic isoform, of RON receptor tyrosine kinase, e.g., human RON receptor tyrosine kinase. For example, the antibody molecule binds specifically to isoform RONΔ160 having an in-frame deletion of about 109 amino acids skipping exons 5 and 6 of the extracellular domain of RON, thus forming a junctional region between exon 4 and exon 7. For example, the antibody molecule binds preferentially to RONΔ160 or a fragment thereof, but does not substantially bind to (e.g., shows less than 10%, 8%, 5%, 4%, 3%, 2%, 1% cross-reactivity with) RON receptor tyrosine kinase (e.g., non-oncogenic human RON receptor tyrosine kinase, e.g., SEQ ID NO: 45). In those embodiments, the antibody molecule binds specifically to at least one epitope found at the junctional region between exon 4 and exon 7 of SEQ ID NO: 12 or a fragment thereof, or an amino acid sequence encoded by a nucleotide sequence of SEQ ID NO: 11 or a fragment thereof; or an amino acid or nucleotide sequence substantially identical thereto.

In yet another embodiment, the antibody molecule binds specifically to an isoform, e.g., an oncogenic isoform, of KIT receptor tyrosine kinase, e.g., human KIT receptor tyrosine kinase. For example, the antibody molecule binds specifically to a KIT isoform having a deletion of exon 11. For example, the antibody molecule binds preferentially to exon 11-deleted KIT isoform (SEQ ID NO: 46) or a fragment thereof, but does not substantially bind to (e.g., shows less than 10%, 8%, 5%, 4%, 3%, 2%, 1% cross-reactivity with) KIT (e.g., non-oncogenic human KIT, e.g., full-length receptor (SEQ ID NO: 47)). In those embodiments, the antibody molecule binds specifically to at least one epitope found at the junctional region between exons 10 and 12 of SEQ ID NO:14 or a fragment thereof, or an amino acid sequence encoded by a nucleotide sequence of SEQ ID NO:13 or a fragment thereof; or an amino acid or nucleotide sequence substantially identical thereto.

In yet another embodiment, the antibody molecule binds specifically to an isoform, e.g., an oncogenic isoform, of PDGF, e.g., human PDGF. For example, the antibody molecule binds specifically to a PDGF isoform having an in-frame deletion of exon 6. For example, the antibody molecule binds preferentially to exon 6-deleted PDGF isoform or a fragment thereof, but does not substantially bind to (e.g., shows less than 10%, 8%, 5%, 4%, 3%, 2%, 1% cross-reactivity with) PDGF (e.g., non-oncogenic human PDGF, e.g., PDGF isoform 1 (SEQ ID NO: 49)). In those embodiments, the antibody molecule binds specifically to at least one epitope found at the junctional region between exons 5 and 7 of SEQ ID NO: 16 or a fragment thereof, or an amino acid sequence encoded by a nucleotide sequence of SEQ ID NO: 15 or a fragment thereof; or an amino acid or nucleotide sequence substantially identical thereto.

In another embodiment, the antibody molecule binds specifically to an isoform, e.g., an oncogenic isoform, of PDGF receptor alpha, e.g., human PDGF receptor alpha. For example, the antibody molecule binds specifically to a PDGFR-alpha isoform having an in-frame deletion of exons 7 and 8. For example, the antibody molecule binds preferentially to exon 7/8-deleted PDGFR-alpha isoform (SEQ ID NO: 51) or a fragment thereof, but does not substantially bind to (e.g., shows less than 10%, 8%, 5%, 4%, 3%, 2%, 1% cross-reactivity with) PDGFR-alpha (e.g., non-oncogenic human PDGFR-alpha, e.g., PDGFR-alpha isoform 1 (SEQ ID NO: 50) In those embodiments, the antibody molecule binds specifically to at least one epitope found at the junctional region between exons 6 and 9 of SEQ ID NO:18 or a fragment thereof, or an amino acid sequence encoded by a nucleotide sequence of SEQ ID NO:17 or a fragment thereof; or an amino acid or nucleotide sequence substantially identical thereto.

The antibody molecule can be a monoclonal or single specificity antibody, or an antigen-binding fragment thereof (e.g., an Fab, F(ab′)₂, Fv, a single chain Fv fragment, a single domain antibody, a diabody (dAb), a bivalent or bispecific antibody or fragment thereof, a single domain variant thereof, or a camelid antibody) that binds to an isoform (e.g., an oncogenic isoform) polypeptide or a fragment or an epitope thereof as described herein. Typically, the antibody molecule is a human, humanized, chimeric, camelid or in vitro generated antibody to an isoform polypeptide or a fragment or an epitope thereof as described herein. The antibody molecule can be full-length (e.g., can include at least one, and typically two, complete heavy chains, and at least one, and typically two, complete light chains) or can include an antigen-binding fragment (e.g., a Fab, F(ab′)₂, Fv, a single chain Fv fragment, or a single domain antibody or fragment thereof). In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function and/or complement function). In one embodiment, the constant region is altered to increase Fc receptor binding, effector cell function and/or complement fixation). For example, the constant region is mutated at positions 296 (M to Y), 298 (S to T), 300 (T to E), 477 (H to K) and 478 (N to F) of SEQ ID NO: 55 to increase Fc receptor binding.

In embodiments, the antibody molecule inhibits, reduces or neutralizes one or more activities of the isoforms, e.g., oncogenic isoforms, in a hyperproliferative (e.g., cancerous or tumor) cell and/or tissue. For example, the antibody molecule may selectively and specifically reduce or inhibit an oncogenic isoform-associated activity chosen from one or more of: (i) binding of a ligand or co-receptor (e.g., FGF ligand (e.g., FGF8b, FGF2, FGF17 or FGF18)) to FGFR2 isoform IIIc); (ii) receptor dimerization (e.g., FGFR2 isoform IIIc dimerization); (iii) receptor signaling, e.g., FGFR2 isoform IIIc signaling; (iv) hyperproliferative (e.g., cancerous or tumor) cell proliferation, growth and/or survival, for example, by induction of apoptosis of the hyperproliferative cell; and/or (v) angiogenesis and/or vascularization of a tumor. In certain embodiments, the antibody molecule is conjugated to one or more cytotoxic or cytostatic agents or moieties, e.g., a therapeutic drug; a compound emitting radiation; molecules of plant, fungal, or bacterial origin, or a biological protein (e.g., a protein toxin); or a particle (e.g., a recombinant viral particle, e.g., via a viral coat protein). Upon binding of the conjugated antibody molecule to an epitope located on an exon sequence or a junctional region predominantly expressed and/or associated with one or more cancerous or tumor cells or disorders (e.g., an epitope as described herein), the conjugated antibody molecule selectively targets or delivers the cytotoxic or cytostatic agent to the hyperproliferative (e.g., cancerous or tumor) cell and/or tissue. In other embodiments, the antibody molecule can be used alone in unconjugated form to thereby reduce an activity (e.g., cell growth or proliferation) and/or kill the hyperproliferative (e.g., cancerous or tumor) cell and/or tissue by, e.g., antibody-dependent cell killing mechanisms, such as complement-mediated cell lysis and/or effector cell-mediated cell killing. In other embodiments, the antibody molecule can disrupt a cellular interaction, e.g., binding of the isoform, e.g., the oncogenic isoform, to a cognate receptor or ligand, thereby reducing or blocking the activity of the hyperproliferative (e.g., cancerous or tumor) cell and/or tissue. For example, the antibody molecule that selectively binds to exon IIIc of FGFR2 can reduce or inhibit the interaction of FGFR2 isoform IIIc to one or more of its ligands, e.g., one or more of: FGF8b, FGF2, FGF17 or FGF18, thus reducing the proliferation and/or survival of FGFR2 isoform IIIc-expressing cells.

In other embodiments, the isoform-specific inhibitor is a full length or a fragment of an isoform receptor polypeptide, e.g., an inhibitory ligand-binding domain of an isoform receptor polypeptide. For example, the isoform-binding molecule can be a soluble form of an FGFR2 isoform IIIc receptor (e.g., a soluble form of mammalian (e.g., human) FGFR2 isoform IIIc comprising a ligand (e.g., FGF)-binding domain. For example, the isoform-specific inhibitor can include about amino acids 1 to 262 of human FGFR2 isoform IIIc receptor (FIG. 13C; amino acids 1-262 of SEQ ID NO: 55 (includes signal peptide)); or an amino acid sequence substantially identical thereto. Alternatively, the isoform-specific inhibitor can include an amino acid sequence encoded by the nucleotide sequence from about nucleotides 1 to 786 of human FGFR2 isoform IIIc (FIG. 13B; nucleotides 1-786 of SEQ ID NO: 54); or an amino acid sequence substantially identical thereto.

A soluble form of an isoform receptor polypeptide can be used alone or functionally linked (e.g., by chemical coupling, genetic or polypeptide fusion, non-covalent association or otherwise) to a second moiety, e.g., an immunoglobulin Fc domain, serum albumin, pegylation, a GST, Lex-A or an MBP polypeptide sequence. The fusion proteins may additionally include a linker sequence joining the first moiety, e.g., a soluble isoform receptor polypeptide, to the second moiety. In other embodiments, additional amino acid sequences can be added to the N- or C-terminus of the fusion protein to facilitate expression, steric flexibility, detection and/or isolation or purification. For example, a soluble form of an isoform receptor polypeptide can be fused to a heavy chain constant region of the various isotypes, including: IgG1, IgG2, IgG3, IgG4, IgM, IgA1, IgA2, IgD, and IgE). For example, the fusion protein can include the extracellular domain of a human FGFR2 isoform IIIc receptor (or a sequence homologous thereto), and, e.g., fused to, a human immunoglobulin Fc chain, e.g., human IgG (e.g., human IgG1 or human IgG2, or a mutated form thereof). The Fc sequence can be mutated at one or more amino acids to enhance or reduce effector cell function, Fc receptor binding and/or complement activity. One exemplary fusion protein that includes the amino acid sequence from about amino acids 1 to 262 of human FGFR2 isoform IIIc receptor (FIG. 13C; amino acids 1-262 of SEQ ID NO: 55) fused via an Arg-Ser linker to a human IgG1 Fc is shown in FIG. 13C (SEQ ID NO: 55).

In yet another embodiment, the isoform-specific inhibitor includes a peptide or a functional variant thereof (e.g., a functional analog or derivative thereof). In some embodiments, the peptide or functional variant thereof consists of, or includes, an amino acid sequence located at the junctional region between two exons that are predominantly joined together in protein isoforms expressed or associated with one or more cancerous or tumor cells or disorders, e.g., as a result of an in-frame exon deletion or the use of an alternatively spliced exon. In one embodiment, the peptide or functional variant thereof consists of, or includes, an amino acid sequence, up to 60 amino acids or less (e.g., up to 50, 40, 30, 20, 10 or less amino acids), and which is identical to the alternative spliced form of Exon III, e.g., from about amino acids 301 to 360 of FGFR2-IIIc (SEQ ID NO:2); about amino acids 314 to 324 of FGFR2-IIIc (AAGVNTTDKEI, SEQ ID NO:4); about amino acids 328 to 337 of FGFR2-IIIc (YIRNVTFEDA, SEQ ID NO:6); about amino acids 350 to 353 of FGFR2-IIIc (ISFH, SEQ ID NO:8), or an amino acid sequence encoded by a nucleotide sequence of SEQ ID NOs: 1, 3, 5 or 7; or an amino acid or nucleotide sequence substantially identical thereto. In another embodiment, the peptide or functional variant thereof consists of, or includes, an amino acid sequence, up to 60 amino acids or less (e.g., up to 50, 40, 30, 20, 10 or less amino acids), and which is identical the junctional region between Ig-II and Ig-III of FGFR1L (SEQ ID NO:10) or a fragment thereof, or an amino acid sequence encoded by a nucleotide sequence of SEQ ID NO:9 or a fragment thereof; or an amino acid or nucleotide sequence substantially identical thereto. In yet other embodiments, the peptide or functional variant thereof consists of, or includes, an amino acid sequence, up to 60 amino acids or less (e.g., up to 50, 40, 30, 20, 10 or less amino acids), and which is identical to the junctional region between exon 4 and exon 7 of isoform RONΔ160 (SEQ ID NO:12) or a fragment thereof, or an amino acid sequence encoded by a nucleotide sequence of SEQ ID NO:11 or a fragment thereof; or an amino acid or nucleotide sequence substantially identical thereto. In yet another embodiment, the peptide or functional variant thereof consists of, or includes, an amino acid sequence, up to 60 amino acids or less (e.g., up to 50, 40, 30, 20, 10 or less amino acids), and which is identical to the junctional region of KIT between exons 10 and 12 of SEQ ID NO:14 or a fragment thereof, or an amino acid sequence encoded by a nucleotide sequence of SEQ ID NO:13 or a fragment thereof; or an amino acid or nucleotide sequence substantially identical thereto. In yet another embodiment, the peptide or functional variant thereof consists of, or includes, an amino acid sequence, up to 60 amino acids or less (e.g., up to 50, 40, 30, 20, 10 or less amino acids), and which is identical to the junctional region of PDGF between exons 5 and 7 of SEQ ID NO:16 or a fragment thereof, or an amino acid sequence encoded by a nucleotide sequence of SEQ ID NO:15 or a fragment thereof; or an amino acid or nucleotide sequence substantially identical thereto. In another embodiment, the peptide or functional variant thereof consists of, or includes, an amino acid sequence, up to 60 amino acids or less (e.g., up to 50, 40, 30, 20, 10 or less amino acids), and which is identical to the junctional region of PDGFR-alpha between exons 6 and 9 of SEQ ID NO:18 or a fragment thereof, or an amino acid sequence encoded by a nucleotide sequence of SEQ ID NO:17 or a fragment thereof; or an amino acid or nucleotide sequence substantially identical thereto.

The peptides or a functional variant thereof can be made recombinantly or synthetically, e.g., using solid phase synthesis. The isoform-specific inhibitor may include at least one, or alternatively, two or more peptide or variants thereof as described herein. For example, any combination of two or more peptide or peptide variants can be arranged, optionally, via a linker sequence. The peptides can be functionally linked (e.g., by chemical coupling, genetic fusion, non-covalent association or otherwise) to one or more other molecular entities, e.g., carriers (e.g., an immunoglobulin Fc domain, serum albumin, pegylation, a GST, Lex-A or an MBP polypeptide sequence) to enhance the peptide stability in vivo. Alternatively, the peptides can be modified by, e.g., addition of chemical protecting groups, to enhance the peptide stability in vivo.

It will be understood that the antibody molecules, soluble or fusion proteins, peptides, and nucleic acid inhibitors described herein can be functionally linked or derivatized (e.g., by chemical coupling, genetic fusion, non-covalent association or otherwise) to one or more other molecular entities, such as an antibody (e.g., a bispecific or a multispecific antibody), toxins, radioisotopes, cytotoxic or cytostatic agents, a label, among others. For example, the antibody molecules, soluble or fusion proteins, peptides, and nucleic acid inhibitors described herein can be coupled to a label, such as a fluorescent label, a biologically active enzyme label, a radioisotope (e.g., a radioactive ion), a nuclear magnetic resonance active label, a luminescent label, or a chromophore. In other embodiments, the antibody molecules, soluble or fusion proteins and peptides described herein can be coupled to a therapeutic agent, e.g., a cytotoxic moiety (e.g., a therapeutic drug; a radioisotope: molecules of plant, fungal, or bacterial origin: or biological proteins (e.g., protein toxins); or particles (e.g., recombinant viral particles, e.g., via a viral coat protein); or mixtures thereof. The therapeutic agent can be an intracellularly active drug or other agent, such as short-range radiation emitters, including, for example, short-rage, high-energy α-emitters, as described herein. In some preferred embodiments, the antibody molecules, soluble or fusion proteins and peptides described herein, can be coupled to a molecule of plant or bacterial origin (or derivative thereof), e.g., a maytansinoid, a taxane, or a calicheamicin. A radioisotope can be an α-, β-, or γ-emitter, or an β- and γ-emitter. Radioisotopes useful as therapeutic agents include yttrium (⁹⁰Y), lutetium (¹⁷⁷Lu), actinium (²²⁵Ac), praseodymium, astatine (²¹¹At), rhenium (¹⁸⁶Re), bismuth (²¹²Bi ²¹³Bi), and rhodium (¹⁸⁸Rh). Radioisotopes useful as labels, e.g., for use in diagnostics, include iodine (¹³¹I or ¹²⁵I, indium (¹¹¹In), technetium (⁹⁹mTc), phosphorus (³²P), carbon (¹⁴C), and tritium (³H). The antibody molecules, soluble or fusion proteins and peptides described herein can also be linked to another antibody to form, e.g., a bispecific or a multispecific antibody.

In another embodiment, the isoform-binding molecule inhibits the expression of nucleic acid encoding the isoform, e.g., the oncogenic isoform (e.g., an oncogenic isoform as described herein). Examples of such isoform-binding molecules include nucleic acid molecules, for example, antisense molecules, ribozymes, RNAi, triple helix molecules that hybridize to a nucleic acid encoding the isoform, e.g., the oncogenic isoform, or a transcription regulatory region, and blocks or reduces mRNA expression of the isoform, e.g., the oncogenic isoform.

In another aspect, the invention provides, compositions, e.g., pharmaceutical compositions, which include a pharmaceutically acceptable carrier, excipient or stabilizer, and at least one of the isoform-specific inhibitors described herein. In one embodiment, the isoform-specific inhibitor is conjugated to a label or a therapeutic agent. In one embodiment, the compositions, e.g., the pharmaceutical compositions, comprise a combination of two or more of the aforesaid the isoform-specific inhibitors, or different antibody molecules. For example, a composition, e.g., pharmaceutical composition, which comprises an isoform-specific inhibitor as described herein, in combination with other growth factor inhibitors, such as antibodies against FGF 1-23, FGF receptors 1-4, VEGF, EGF or EGF receptor, PSMA antibody, or Her-2/neu, etc. Combinations of an isoform-specific inhibitor and a drug, e.g., a therapeutic agent (e.g., a cytotoxic or cytostatic drug, e.g., DM1, calicheamicin, or taxanes, topoisomerase inhibitors, or an immunomodulatory agent, e.g., IL-1, 2, 4, 6, or 12, interferon alpha or gamma, or immune cell growth factors such as GM-CSF) are also within the scope of the invention.

The invention also features nucleic acid sequences that encode the isoform-binding molecules described herein described herein. For example, the invention features, a first and second nucleic acid encoding a modified heavy and light chain variable region, respectively, of an antibody molecule as described herein. In other embodiments, the invention provides nucleic acids comprising nucleotide sequences encoding the soluble receptors, fusions, peptides and functional analogs thereof described herein. In another aspect, the invention features host cells and vectors containing the nucleic acids of the invention. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NS0), Chinese hamster ovary cells (CHO), COS cells, oocyte cells, and cells from a transgenic animal, e.g., mammary epithelial cell. For example, nucleic acids encoding the isoform binding molecule described herein can be expressed in a transgenic animal. In one embodiment, the nucleic acids are placed under the control of a tissue-specific promoter (e.g., a mammary specific promoter) and the antibody is produced in the transgenic animal. For example, the isoform binding molecule is secreted into the milk of the transgenic animal, such as a transgenic cow, pig, horse, sheep, goat or rodent.

In one aspect, the invention features a method of providing an isoform binding antibody molecule that specifically binds to an isoform (e.g., an oncogenic isoform) polypeptide. The method includes: providing a isoform-specific antigen (e.g., an antigen comprising at least a portion of an epitope as described herein); obtaining an antibody molecule that specifically binds to the isoform polypeptide; and evaluating if the antibody molecule specifically binds to the isoform polypeptide (e.g., evaluating if there is a decrease in binding between the antibody molecule and the isoform polypeptide in the present of one or more of the epitopes described herein), or evaluating efficacy of the antibody molecule in modulating, e.g., inhibiting, the activity of the isoform (e.g., an oncogenic isoform) polypeptide. The method can further include administering the antibody molecule to a subject, e.g., a human or non-human animal.

Isoform-specific epitopes, e.g., isolated epitopes, as described herein are also encompassed by the present invention. The epitopes can be linear or conformational protein of the isoform (e.g., oncogenic) isoform, e.g., from about 2 to 80, about 4 to 75, about 5 to 70, about 10 to 60, about 10 to 50, about 10 to 40, about 10 to 30, about 10 to 20, amino acid residues. In certain embodiments, the epitope consists of, or includes, an amino acid sequence located at the junctional region between two exons that are predominantly joined together in protein isoforms expressed or associated with one or more cancerous or tumor cells or disorders, e.g., as a result of an in-frame exon deletion or the use of an alternatively spliced exon. For example, the epitope can consist of, or include, an amino acid sequence identical to the alternative spliced form of Exon III, e.g., from about amino acids 301 to 360 of FGFR2-IIIc (SEQ ID NO:2); about amino acids 314 to 324 of FGFR2-IIIc (AAGVNTTDKEI, SEQ ID NO:4); about amino acids 328 to 337 of FGFR2-IIIc (YIRNVTFEDA, SEQ ID NO:6); about amino acids 350 to 353 of FGFR2-IIIc (ISFH, SEQ ID NO:8), or an amino acid sequence encoded by a nucleotide sequence of SEQ ID NOs: 1, 3, 5 or 7; or an amino acid or nucleotide sequence substantially identical thereto. In another embodiment, the epitope consists of, or includes, an amino acid sequence identical the junctional region between Ig-II and Ig-III of FGFR1L (SEQ ID NO: 10) or a fragment thereof, or an amino acid sequence encoded by a nucleotide sequence of SEQ ID NO: 9 or a fragment thereof; or an amino acid or nucleotide sequence substantially identical thereto. In yet other embodiments, the epitope consists of, or includes, an amino acid sequence identical to the junctional region between exon 4 and exon 7 of isoform RONΔ160 (SEQ ID NO: 12) or a fragment thereof, or an amino acid sequence encoded by a nucleotide sequence of SEQ ID NO: 11 or a fragment thereof; or an amino acid or nucleotide sequence substantially identical thereto. In yet another embodiment, the epitope consists of, or includes, an amino acid sequence identical to the junctional region of KIT between exons 10 and 12 of SEQ ID NO: 14 or a fragment thereof, or an amino acid sequence encoded by a nucleotide sequence of SEQ ID NO:13 or a fragment thereof; or an amino acid or nucleotide sequence substantially identical thereto. In yet another embodiment, the epitope consists of, or includes, an amino acid sequence identical to the junctional region of PDGF between exons 5 and 7 of SEQ ID NO: 16 or a fragment thereof, or an amino acid sequence encoded by a nucleotide sequence of SEQ ID NO: 15 or a fragment thereof; or an amino acid or nucleotide sequence substantially identical thereto. In another embodiment, the epitope consists of, or includes, an amino acid sequence identical to the junctional region of PDGFR-alpha between exons 6 and 9 of SEQ ID NO: 18 or a fragment thereof, or an amino acid sequence encoded by a nucleotide sequence of SEQ ID NO: 17 or a fragment thereof; or an amino acid or nucleotide sequence substantially identical thereto.

The invention also features a method of reducing the activity (e.g., cell growth or proliferation), or inducing the killing (e.g., inducing apoptosis of), a hyperproliferative cell, e.g., a cancerous or tumor cell (e.g., a cancerous or tumor cell expressing an oncogenic isoform, such as FGFR2-IIIc and exon deleted-isoforms of FGFR1, RON, KIT, PDGF and PDGFR-alpha, as described herein). The method includes contacting the hyperproliferative cell, or a cell (e.g., a vascular cell) in proximity to the hyperproliferative cell, with one or more isoform-specific inhibitors as described herein, e.g., an isoform-specific antibody molecule described herein, in an amount sufficient to reduce the expression or activity of the isoform, e.g., the oncogenic isoform, thereby reducing the activity of, or killing, the hyperproliferative cell. The isoform-specific inhibitors as described herein can be used in conjugated or unconjugated form, alone as a monotherapy or in combination with one or more therapeutic agents, to thereby kill, or reduce the activity, e.g., inhibit cell growth of, the hyperproliferative cell.

In embodiments, the isoform-binding molecule is an antibody molecule that specifically binds to FGFR2-IIIc, e.g., an antibody molecule that specifically binds to an amino acid sequence identical to the alternative spliced form of Exon III, e.g., from about amino acids 301 to 360 of FGFR2-IIIc (SEQ ID NO:2); about amino acids 314 to 324 of FGFR2-IIIc (AAGVNTTDKEI, SEQ ID NO:4); about amino acids 328 to 337 of FGFR2-IIIc (YIRNVTFEDA, SEQ ID NO:6); about amino acids 350 to 353 of FGFR2-IIIc (ISFH, SEQ ID NO:8), or an amino acid sequence encoded by a nucleotide sequence of SEQ ID NOs: 1, 3, 5 or 7; or an amino acid or nucleotide sequence substantially identical thereto. In such embodiments, the hyperproliferative cell is a cancerous or tumor cell from the prostate, breast, pancreas, ovary, brain (glioblastoma), gastric cancers, lung squamous cell carcinoma, non-small cell lung carcinoma, tyroid cancer, endometrial carcinoma, hematopoietic cancers, and skeletal disorders, such as craniofacial dysostosis 1, Crouzon syndrome, Pfeiffer syndrome, Jackson-Weiss syndrome and Apert syndrome.

The methods can be used on cells in culture, e.g., in vitro or ex vivo. For example, hyperproliferative cells (e.g., cancerous or metastatic cells (e.g., prostatic, renal, urothelial (e.g., bladder), testicular, ovarian, breast, colon, rectal, lung (e.g., non-small cell lung carcinoma), liver, brain, neural (e.g., neuroendocrine), glial (e.g., glioblastoma), pancreatic, melanoma (e.g., malignant melanoma), or soft tissue sarcoma cancerous or metastatic cells) can be cultured in vitro in culture medium and the contacting step can be effected by adding the isoform binding molecule, to the culture medium. Alternatively, the method can be performed on hyperproliferative cells present in a subject, as part of an in vivo (e.g., therapeutic or prophylactic) protocol.

Methods of the invention can be used, for example, to treat or prevent a hyperproliferative disorder, e.g., a cancer (primary, recurring or metastasizing) of, e.g. prostate, breast, pancreas and brain (glioblastoma), by administering to a subject an isoform-specific inhibitor described herein, in an amount effective to treat or prevent such disorder. In one embodiment, the cancer is an adenocarcinoma or carcinoma of the prostate and/or testicular tumors. For example, the cancer is hormone-resistant or refractory prostate cancer. In one embodiment, the cancer is an androgen-resistant or refractory prostate cancer associated with elevated expression of FGFR2-IIIc. For example, the cancer shows elevated level or expression of FGFR2-IIIc protein or mRNA compared to a reference value (e.g., a non-cancerous prostatic tissue), optionally, accompanied by a reduction in one or more epithelial markers (e.g., reduction in the level or expression of epithelial cell surface adhesion molecules (Ep-CAM) and/or gain of mesenchymal markers. In certain embodiments, the cancer is a metastatic cancer showing elevated levels of prostate-derived circulating tumor cells (e.g., prostate-derived circulating FGFR2IIIc-expressing prostatic tumor cells). Methods and compositions disclosed herein are particularly useful for treating metastatic lesions associated with prostate cancer. In some embodiments, the patient will have undergone one or more of prostatectomy, chemotherapy, or other anti-tumor therapy and the primary or sole target will be metastatic lesions, e.g., metastases in the bone marrow or lymph nodes.

In other embodiments, the cancer treated with the isoform-specific inhibitor(s) described herein includes, but is not limited to, solid tumors, soft tissue tumors, and metastatic lesions. Examples of solid tumors include malignancies, e.g., sarcomas, adenocarcinomas, and carcinomas, of the various organ systems, such as those affecting lung, breast, lymphoid, gastrointestinal (e.g., colon), genitals and genitourinary tract (e.g., renal, urothelial, bladder cells), pharynx, CNS (e.g., brain, neural or glial cells), skin (e.g., melanoma), and pancreas, as well as adenocarcinomas which include malignancies such as most colon cancers, rectal cancer, renal-cell carcinoma, liver cancer, non-small cell-carcinoma of the lung, cancer of the small intestine and cancer of the esophagus. Methods and compositions disclosed herein are particularly useful for treating metastatic lesions associated with the aforementioned cancers. In some embodiments, the patient will have undergone one or more of surgical removal of a tissue, chemotherapy, or other anti-cancer therapy and the primary or sole target will be metastatic lesions, e.g., metastases in the bone marrow or lymph nodes. For example, a reduction in expression or activity of an FGFR2-IIIc oncogenic isoform can be used to prevent and/or treat hormone-refractory prostate cancer, breast cancer, bladder cancer, thyroid cancer, or other form of cancer. A reduction in expression or activity of FGFR1L can be used to prevent and/or treat pancreatic adenocarcinoma, prostate cancer, or other form of cancer. A reduction in expression or activity of a RON receptor tyrosine kinase Δ160 isoform may be used to prevent and/or treat metastatic colorectal cancer, breast cancer, ovarian cancer, lung cancer, bladder cancer, or other form of cancer. A reduction in expression or activity of a KIT receptor tyrosine kinase oncogenic isoform can be used to prevent and/or treat gastrointestinal stromal tumors (GISTs) or other form of cancer. A reduction in expression or activity of a PDGFR-alpha isoform can be used to prevent and/or treat brain cancer, glioblastoma, prostate cancer, bone metastasis, GIST, or other form of cancer.

In one embodiment, the subject is treated to prevent a hyperproliferative disorder, e.g., a hyperproliferative disorder as described herein. The subject can be a mammal, e.g., a primate, preferably a higher primate, e.g., a human (e.g., a patient having, or at risk of, a hyperproliferative disorder described herein, e.g., a prostatic cancer disorder). In one embodiment, the subject is a patient having prostate cancer (e.g., a patient suffering from recurrent or metastatic prostate cancer). The subject can be one at risk for the disorder, e.g., a subject having a relative afflicted with the disorder, e.g., a subject with one or more of a grandparent, parent, uncle or aunt, sibling, or child who has or had the disorder, or a subject having a genetic trait associated with risk for the disorder. In one embodiment, the subject can be symptomatic or asymptomatic. For example, the subject can suffer from symptomatic or asymptomatic prostatic cancer, e.g., hormone-resistant or refractory prostate cancer. In some embodiments, the subject suffers from metastatic prostate cancer. In some embodiments, the subject has elevated levels of prostate-derived circulating tumor cells (e.g., prostate-derived circulating FGFR2IIIc-expressing prostatic tumor cells). In other embodiments, the subject has abnormal levels of one or more markers for a cancer, e.g., prostatic cancer. For example, the subject has abnormal levels of prostate-specific antigen (PSA), prostate specific membrane antigen (PSMA), prostate stem cell antigen (PSCA), androgen receptor (AR), chromogranin, synaptophysin, MIB-1, and/or α-methylacyl-CoA racemase (AMACR).

The isoform-specific inhibitors described herein can be administered to the subject systemically (e.g., orally, parenterally, subcutaneously, intravenously, rectally, intramuscularly, intraperitoneally, intranasally, transdermally, or by inhalation or intracavitary installation), topically, or by application to mucous membranes, such as the nose, throat and bronchial tubes.

The methods of the invention, e.g., methods of treatment or preventing, can further include the step of monitoring the subject, e.g., for a change (e.g., an increase or decrease) in one or more of: tumor size; levels of a cancer marker (e.g., level or expression of FGFR2IIIc; levels of circulating prostate-derived FGFR2IIIc-expressing cells, epithelial cell markers (Ep-CAM), FGF ligands (e.g., FGF8), stromal derived factor α (SDFα), VEGF (e.g., VEGF121), mesenchymal markers, PSA, PSMA, PSCA, AR, chromogranin, synaptophysin, MIB-1, AMACR, alkaline phosphatase, and/or serum hemoglobin for a patient with prostate cancer); the rate of appearance of new lesions, e.g., in a bone scan; the appearance of new disease-related symptoms; the size of soft tissue mass, e.g., a decreased or stabilization; quality of life, e.g., amount of disease associated pain, e.g., bone pain; or any other parameter related to clinical outcome. The subject can be monitored in one or more of the following periods: prior to beginning of treatment; during the treatment; or after one or more elements of the treatment have been administered. Monitoring can be used to evaluate the need for further treatment with the same isoform-binding molecule or for additional treatment with additional agents. Generally, a decrease in one or more of the parameters described above is indicative of the improved condition of the subject, although with serum hemoglobin levels, an increase can be associated with the improved condition of the subject.

The methods of the invention can further include the step of analyzing a nucleic acid or protein from the subject, e.g., analyzing the genotype of the subject. In one embodiment, a nucleic acid encoding the isoform, e.g., the oncogenic isoform, and/or an upstream or downstream component(s) of the isoform signalling, e.g., an extracellular or intracellular activator or inhibitor of the isoform, is analyzed. The analysis can be used, e.g., to evaluate the suitability of, or to choose between alternative treatments, e.g., a particular dosage, mode of delivery, time of delivery, inclusion of adjunctive therapy, e.g., administration in combination with a second agent, or generally to determine the subject's probable drug response phenotype or genotype. The nucleic acid or protein can be analyzed at any stage of treatment, but preferably, prior to administration of the isoform-specific inhibitor to thereby determine appropriate dosage(s) and treatment regimen(s) of the isoform-specific inhibitor (e.g., amount per treatment or frequency of treatments) for prophylactic or therapeutic treatment of the subject.

The isoform-specific inhibitor (e.g., the isoform-specific binding agent) can be used alone in unconjugated form to thereby reduce the activity or induce the killing of the isoform-expressing hyperproliferative or cancerous cells by, e.g., antibody-dependent cell killing mechanisms such as complement-mediated cell lysis and/or effector cell-mediated cell killing. In other embodiments, the isoform-specific inhibitor can be bound to a substance, e.g., a cytotoxic agent or moiety (e.g., a therapeutic drug; a compound emitting radiation; molecules of plant, fungal, or bacterial origin; or a biological protein (e.g., a protein toxin) or particle (e.g., a recombinant viral particle, e.g., via a viral coat protein). For example, the isoform-specific inhibitor can be coupled to a radioactive isotope such as an α-, β-, or γ-emitter, or β- and γ-emitter. Examples of radioactive isotopes include iodine (¹³¹I or ¹²⁵I, yttrium (⁹⁰Y), lutetium (¹⁷⁷Lu), actinium (²²⁵Ac), praseodymium, or bismuth (²¹²Bi or ²¹³Bi). Alternatively, the isoform-binding molecule can be coupled to a biological protein, a molecule of plant or bacterial origin (or derivative thereof), e.g., a maytansinoid (e.g., maytansinol or DM1), as well as a taxane (e.g., taxol or taxotere), or calicheamicin. The maytansinoid can be, for example, maytansinol or a maytansinol analogue. Examples of maytansinol analogues include those having a modified aromatic ring (e.g., C-19-decloro, C-20-demethoxy, C-20-acyloxy) and those having modifications at other positions (e.g., C-9-CH, C-14-alkoxymethyl, C-14-hydroxymethyl or aceloxymethyl, C-15-hydroxy/acyloxy, C-15-methoxy, C-18-N-demethyl 4,5-deoxy). Maytansinol and maytansinol analogues are described, for example, in U.S. Pat. No. 6,333,410, the contents of which is incorporated herein by reference. The calicheamicin can be, for example, a bromo-complex calicheamicin (e.g., an alpha, beta or gamma bromo-complex), an iodo-complex calicheamicin (e.g., an alpha, beta or gamma iodo-complex), or analogs and mimics thereof. Bromo-complex calicheamicins include α₁-BR, α₂-BR, α₃-BR, α₄-BR, β₁-BR, β₂-BR and γ₁-BR. Iodo-complex calicheamicins include α₁-I, α₂-I, α₃-I, β₁-I, β₂-I, δ₁-I and γ₁-BR. Calicheamicin and mutants, analogs and mimics thereof are described, for example, in U.S. Pat. No. 4,970,198, issued Nov. 13, 1990, U.S. Pat. No. 5,264,586, issued Nov. 23, 1993, U.S. Pat. No. 5,550,246, issued Aug. 27, 1996, U.S. Pat. No. 5,712,374, issued Jan. 27, 1998, and U.S. Pat. No. 5,714,586, issued Feb. 3, 1998, the contents of which are incorporated herein by reference. Maytansinol can be coupled to antibodies using, e.g., an N-succinimidyl 3-(2-pyridyldithio)proprionate (also known as N-succinimidyl 4-(2-pyridyldithio)pentanoate or SPP), 4-succinimidyl-oxycarbonyl-a-(2-pyridyldithio)-toluene (SMPT), N-succinimidyl-3-(2-pyridyldithio)butyrate (SDPB), 2-iminothiolane, or S-acetylsuccinic anhydride.

The methods and compositions of the invention can be used in combination with other therapeutic modalities. In one embodiment, the methods of the invention include administering to the subject an isoform-specific inhibitor as described herein, in combination with a cytotoxic agent, in an amount effective to treat or prevent said disorder. The binding molecule and the cytotoxic agent can be administered simultaneously or sequentially. In other embodiments, the methods and compositions of the invention are used in combination with surgical and/or radiation procedures. In yet other embodiments, the methods can be used in combination with immunodulatory agents, e.g., IL-1, 2, 4, 6, or 12, or interferon alpha or gamma, or immune cell growth factors such as GM-CSF. Exemplary cytotoxic agents that can be administered in combination with the isoform-specific inhibitor include antimicrotubule agents, topoisomerase inhibitors, antimetabolites, mitotic inhibitors, alkylating agents, intercalating agents, agents capable of interfering with a signal transduction pathway, agents that promote apoptosis and radiation.

In therapies of prostatic disorders, e.g., prostate cancer, the isoform-specific inhibitor can be used in combination with existing therapeutic modalities, e.g., prostatectomy (partial or radical), radiation therapy, hormonal therapy, androgen ablation therapy, and cytotoxic chemotherapy. Typically, hormonal therapy works to reduce the levels of androgens in a patient, and can involve administering a leuteinizing hormone-releasing hormone (LHRH) analog or agonist (e.g., Lupron, Zoladex, leuprolide, buserelin, or goserelin), as well as antagonists (e.g., Abarelix). Non-steroidal anti-androgens, e.g., flutamide, bicalutimade, or nilutamide, can also be used in hormonal therapy, as well as steroidal anti-androgens (e.g., cyproterone acetate or megastrol acetate), estrogens (e.g., diethylstilbestrol), surgical castration, PROSCAR®, secondary or tertiary hormonal manipulations (e.g., involving corticosteroids (e.g., hydrocortisone, prednisone, or dexamnethasone), ketoconazole, and/or aminogluthethimide), inhibitors of 5a-reductase (e.g., finisteride), herbal preparations (e.g., PC-SPES), hypophysectomy, and adrenalectomy. Furthermore, hormonal therapy can be performed intermittently or using combinations of any of the above treatments, e.g., combined use of leuprolide and flutamide.

Any combination and sequence of isoform-specific inhibitor and other therapeutic modalities can be used. The isoform-specific inhibitor and other therapeutic modalities can be administered during periods of active disorder, or during a period of remission or less active disease. The isoform-specific inhibitor and other therapeutic modalities can be administered before treatment, concurrently with treatment, posttreatment, or during remission of the disorder.

In another aspect, the invention features methods for detecting the presence of an isoform (e.g., an oncogenic isoform as described herein) polypeptide or gene expression product in a sample in vitro (e.g., a biological sample, e.g., serum, semen or urine, or a tissue biopsy, e.g., from a hyperproliferative or cancerous lesion). The subject method can be used to evaluate (e.g., monitor treatment or progression of, diagnose and/or stage a disorder described herein, e.g., a hyperproliferative or cancerous disorder, in a subject). The method includes: (i) contacting the sample (and optionally, a reference, e.g., a control sample) with an isoform binding molecule (e.g., an antibody molecule), as described herein, under conditions that allow interaction of the isoform binding molecule and the polypeptide or gene expression product to occur, and (ii) detecting formation of a complex between the isoform binding molecule, and the sample (and optionally, the reference, e.g., control, sample). Formation of the complex is indicative of the presence of the polypeptide or gene expression product, and can indicate the suitability or need for a treatment described herein. For example, a statistically significant change in the formation of the complex in the sample relative to the reference sample, e.g., the control sample, is indicative of the presence of the isoform, e.g., the oncogenic isoform, in the sample. In some embodiments, the methods can include the use of more than one isoform-binding molecules, e.g., two antibody molecules that bind to different epitopes on the same oncogenic isoform (e.g., FGFR2 isoform IIIc) or different oncogenic isoform. For example, the method can involve an immunohistochemistry, immunocytochemistry, FACS, antibody molecule complexed magnetic beads, ELISA assays, PCR-techniques (e.g., RT-PCR), e.g., as described in the appended Examples.

In yet another aspect, the invention provides a method for detecting the presence of an isoform (e.g., an oncogenic isoform as described herein) polypeptide or gene expression product in vivo (e.g., in vivo imaging in a subject). The method can be used to evaluate (e.g., monitor treatment or progression of, diagnose and/or stage a disorder described herein, e.g., a hyperproliferative or cancerous disorder), in a subject, e.g., a mammal, e.g., a primate, e.g., a human. The method includes: (i) administering to a subject an isoform binding molecule (e.g., an antibody molecule as described herein), under conditions that allow interaction of the isoform binding molecule and the polypeptide or gene expression product to occur; and (ii) detecting formation of a complex between the isoform binding molecule and the polypeptide or gene expression product. A statistically significant change in the formation of the complex in the subject relative to the reference, e.g., the control subject or subject's baseline, is indicative of the presence of the polypeptide or gene expression product.

In other embodiments, a method of evaluating (e.g., monitoring treatment or progression of, diagnosing and/or staging a hyperproliferative or cancerous disorder as described herein, in a subject, is provided. The method includes: (i) identifying a subject having, or at risk of having, the disorder, (ii) obtaining a sample of a tissue or cell affected with the disorder, (iii) contacting said sample or a control sample with an isoform binding molecule as described herein, e.g., an antibody molecule as described herein, under conditions that allow an interaction of the binding molecule and the isoform polypeptide or gene product to occur, and (iv) detecting formation of a complex. A statistically significant increase in the formation of the complex with respect to a reference sample, e.g., a control sample, is indicative of the disorder or the stage of the disorder.

Typically, the isoform binding molecule used in the in vivo and in vitro diagnostic methods is directly or indirectly labeled with a detectable substance to facilitate detection of the bound or unbound binding agent. Suitable detectable substances include various biologically active enzymes, prosthetic groups, fluorescent materials, luminescent materials, paramagnetic (e.g., nuclear magnetic resonance active) materials, and radioactive materials. In some embodiments, the isoform binding molecule is coupled to a radioactive ion, e.g., indium (¹¹¹In), iodine (¹³¹I or ¹²⁵I), yttrium (⁹⁰Y) lutetium (¹⁷⁷Lu), actinium (²²⁵Ac), bismuth (²¹²Bi or ²¹³Bi), sulfur (³⁵S), carbon (¹⁴C), tritium (³H), rhodium (¹⁸⁸Rh), technetium (99mTc), praseodymium, or phosphorous (³²P).

The detection/diagnostic methods described herein can further include the step of monitoring the subject, e.g., for a change (e.g., an increase or decrease) in one or more of: tumor size; levels of a cancer marker (e.g., level or expression of FGFR2IIIc; levels of circulating prostate-derived FGFR2IIIc-expressing cells, epithelial cell markers (Ep-CAM), FGF ligands (e.g., FGF8), stromal derived factor alpha (SDFalpha, VEGF (e.g., VEGF121), mesenchymal markers, PSA, PSMA, PSCA, AR, chromogranin, synaptophysin, MIB-1, AMACR, alkaline phosphatase, and/or serum hemoglobin for a patient with prostate cancer); the rate of appearance of new lesions, e.g., in a bone scan; the appearance of new disease-related symptoms; the size of soft tissue mass, e.g., a decreased or stabilization; quality of life, e.g., amount of disease associated pain, e.g., bone pain; or any other parameter related to clinical outcome. The subject can be monitored in one or more of the following periods: prior to beginning of treatment; during the treatment; or after one or more elements of the treatment have been administered. Monitoring can be used to evaluate the need for further treatment with the same isoform-binding molecule or for additional treatment with additional agents. Generally, a decrease in one or more of the parameters described above is indicative of the improved condition of the subject, although with serum hemoglobin levels, an increase can be associated with the improved condition of the subject.

In another aspect, the invention features diagnostic or therapeutic kits that include the isoform-specific inhibitors described herein and instructions for use.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

The terms “proteins” and “polypeptides” are used interchangeably herein.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

All publications, patent applications, patents, and other references mentioned herein are incorporated by reference in their entirety.

Other features, objects, and advantages of the invention will be apparent from the description and drawings, and from the claims.

### BRIEF DESCRIPTION OF DRAWINGS

FIG. 1 depicts the isoform structure of FGFR2 receptor tyrosine kinase. Top: Isoform IIIb is expressed on normal prostate epithelial cells. Bottom: Isoform IIIc is expressed in hormone-refractory prostate cancer. TM=Transmembrane; AB=Acid box; I, II, or III=Ig-like loop I, II, or III.

FIG. 2 depicts the sequence alignment of IIIc (SEQ ID NO: 2) and IIIb isoforms (SEQ ID NO: 65).

FIG. 3A depicts the amino acid sequence of human FGFR2 IIc (SEQ ID NO: 19).

FIG. 3B depicts the nucleotide sequence of human FGFR2 IIc (SEQ ID NO: 20).

FIG. 4A depicts the nucleotide sequence of FGFR2 Exon-IIIc (SEQ ID NO: 1).

FIG. 4B depicts the nucleotide sequence of FGFR2 Exon-IIIb (SEQ ID NO: 64).

FIG. 5A depicts the amino acid (SEQ ID NO: 4) and nucleotide (SEQ ID NO: 3) sequences of peptide IIIc-314.

FIG. 5B depicts the amino acid (SEQ ID NO: 6) and nucleotide (SEQ ID NO: 5) sequences of peptide IIIc-328.

FIG. 5C depicts the amino acid (SEQ ID NO: 8) and nucleotide (SEQ ID NO: 7) sequences of peptide IIIc-350.

FIG. 6A depicts the amino acid (SEQ ID NO: 56) and nucleotide (SEQ ID NO: 60) sequences of IIIb (Loop3-C′) fragment: amino acids 314-351.

FIG. 6B depicts the amino acid (SEQ ID NO: 57) and nucleotide (SEQ ID NO: 61) sequences of IIIb epitope: amino acids 314-328.

FIG. 6C depicts the amino acid (SEQ ID NO: 58) and nucleotide (SEQ ID NO: 62) sequences of IIIb epitope: amino acids 340-351.

FIG. 7 depicts the isoform structure of FGFR1.

FIG. 8 depicts the nucleotide (SEQ ID NO: 9) and amino acid (SEQ ID NO: 10) sequences of FGFR1L epitope sequence at the junction.

FIG. 9 depicts the nucleotide (SEQ ID NO: 11) and amino acid (SEQ ID NO: 12) sequences of RONΔ160 epitope at the junction between exon 4 and exon 7.

FIG. 10 depicts the nucleotide (SEQ ID NO: 13) and amino acid (SEQ ID NO: 14) sequences of the epitope designed for antibody targeting KIT isoform.

FIG. 11 depicts the nucleotide (SEQ ID NO: 15) and amino acid (SEQ ID NO: 16) sequences of the epitope designed for antibody targeting PDGF isoform.

FIG. 12 depicts the nucleotide (SEQ ID NO: 17) and amino acid (SEQ ID NO: 18) sequences of the epitope of PDGFR-alpha isoform.

FIG. 13A depicts the structure of the soluble FGFR2 IIIc-Fc fusion protein.

FIG. 13B depicts the nucleotide sequence (SEQ ID NO: 54) of the soluble FGFR2 IIIc-Fc fusion protein.

FIG. 13C depicts the amino acid sequence (SEQ ID NO: 55) of the soluble FGFR2 IIIc-Fc fusion protein. The signal peptide corresponds to amino acids 1 to 21 of SEQ ID NO: 55.

FIG. 13D depicts a Western blot of SDS-PAGE analysis of CHO stable cell lines expressing the recombinant fusion protein of soluble FGFR2 IIIc-Fc.

FIG. 14 depicts sequence alignments of FGFR2 receptor Ig-like loop-3 regions from human and rat. The C-terminal half of loop-3 is encoded by either exon-8 to give rise to IIIc (shown in bold) (SEQ ID NOs: 2 and 67), or exon-9 to give rise to IIIb (italic) (SEQ ID NOs: 65 and 68). Human and rat sequences are 100% identical in these regions.

FIG. 15 depicts the dual targeting strategy for FGFR2 receptor. Antibody Ab-1 targets the extracellular ligand binding site of the receptor; and TKI (e.g., RO4383596 or Pazopanib) targets the intracellular tyrosine kinase domain.

FIG. 16 depicts the isoform specific primers for PCR analysis of FGFR2 IIc and IIIb.

FIG. 17A depicts the amino acid sequence of human FGFR2 gene (SEQ ID NO: 32).

FIGS. 17B-17C depict the amino acid (SEQ ID NO: 21) and nucleotide sequences (SEQ ID NO: 63) of human FGFR2IIIb, respectively.

FIGS. 17D-17O depict the amino acid sequence of human FGFR2 isoform 4 (SEQ ID NO: 22), isoform 7 (SEQ ID NO: 23), isoform 9 (SEQ ID NO: 24), isoform 10 (SEQ ID NO: 25), isoform 11 (SEQ ID NO: 26), isoform 12 (SEQ ID NO: 27), isoform 13 (SEQ ID NO: 28), isoform 14 (SEQ ID NO: 29), isoform 15 (SEQ ID NO: 30), isoform 17 (SEQ ID NO: 31), isoform 18 (SEQ ID NO: 52), and isoform 19 (SEQ ID NO: 53), respectively.

FIG. 18A depicts the amino acid sequence of human FGFR1 gene (SEQ ID NO: 33).

FIGS. 18B-18H depict the amino acid sequences of human FGFR1 isoform 1 (SEQ ID NO: 38), isoform 4 (SEQ ID NO: 39), isoform 14 (SEQ ID NO: 40), isoform 16 (SEQ ID NO: 41), isoform 17 (SEQ ID NO: 42), isoform 3 (SEQ ID NO: 43), and isoform 18 (SEQ ID NO: 44), respectively.

FIG. 19A depicts the amino acid sequence of human RON gene (SEQ ID NO: 34).

FIG. 19B depicts the amino acid sequence of human non-oncogenic RON isoform (SEQ ID NO: 45).

FIG. 20A depicts the amino acid sequence of human KIT gene (SEQ ID NO: 35).

FIG. 20B depicts the amino acid sequence of human KIT variant with deletion in exon 11 (SEQ ID NO: 46).

FIG. 20C depict the amino acid sequence of full-length human KIT (SEQ ID NO: 47).

FIG. 21A depicts the amino acid sequence of human PDGF gene (SEQ ID NO: 36).

FIG. 21B depicts the amino acid sequence of human PDGF isoform 2 (SEQ ID NO: 48).

FIG. 21C depict the amino acid sequence of full-length human PDGF (SEQ ID NO: 49).

FIG. 22A depicts the amino acid sequence of human PDGFR alpha gene (SEQ ID NO: 37).

FIG. 22B depicts the amino acid sequence of human PDGFR alpha isoform 1(SEQ ID NO: 50).

FIG. 22C depict the amino acid sequence of human PDGFR alpha isoform with deletion in exons 7-8 (SEQ ID NO: 51).

FIG. 23 depicts the amino acid sequence of human FGF8 (SEQ ID NO: 66)

### DETAILED DESCRIPTION

The present invention provides, at least in part, isoform-specific inhibitors that inhibit or reduce one or more isoform-associated activities. In certain embodiments, the isoforms (e.g., polypeptide or nucleic acid isoforms) are expressed and/or are associated with oncogenic or malignant phenotypes (referred to herein as “oncogenic isoforms”). For example, the isoforms can arise from, e.g., one or more of: alternative splicing, frameshifting, translational and/or post-translational events, thereby resulting in different transcription or translation products. In one embodiment, the isoform-specific inhibitor is an isoform-binding molecule, e.g., an antibody molecule, or a nucleic acid inhibitor. In another embodiment, the isoform-specific inhibitor is a soluble receptor polypeptide and a fusion form thereof, or a peptide and a functional variant thereof. For example, the isoform-specific inhibitor can be an oncogenic isoform-binding molecule, e.g., an antibody molecule or a nucleic acid inhibitor that specifically interacts with, e.g., binds to, one or more oncogenic isoforms (e.g., oncogenic isoform polypeptides or nucleic acids encoding the same). In another embodiment, the isoform-specific inhibitor is a soluble receptor polypeptide or a fusion form thereof, or a peptide or a functional variant thereof that reduces or inhibits one or more isoform- (e.g., oncogenic isoform-) associated activities. In embodiments, the soluble receptor or fusion reduce or inhibit (e.g., competitively inhibit) an interaction of the isoform (e.g., the oncogenic isoform) polypeptide and its cognate ligand or receptor.

The oncogenic isoforms can arise from, e.g., alternative splicing, frameshifting, translational and/or post-translational events, of various proto-oncogene expression products in a cell, e.g., a hyperproliferative cell (e.g., a cancerous or tumor cell). The isoform-binding molecules described herein bind to such oncogenic isoforms, but do not substantially bind a predominantly non-oncogenic sequence of the proto-oncogene from which the isoform is derived.

The term “isoform” in the context of a protein or polypeptide as used herein refers to polymers of amino acids of any length that can be derived from one or more of alternative splicing, frameshifting, translational and/or post-translational events. Alternative splicing events include processes (during transcription) by which one or more alternative exons (i.e., portion of a gene that codes for a protein) within a given RNA molecule are combined (by RNA Polymerase molecules) to yield different mRNAs from the same gene. Each such mRNA is known as a “gene transcript”. Commonly, a single gene can encode several different mRNA transcripts, caused by cell- or tissue-specific combination of different exons. For example, multiple forms of fibroblast growth factor receptor 1-3 (FGFR1-3) are known to be generated by alternative splicing of the mRNAs. A frequent splicing event involving FGFR1 and 2 results in receptors containing three immunoglobulin (ig) domains, commonly referred to the α isoform, or only Immunoglobulin II (IgII) and IgIII, referred to as the β isoform. The α isoform has been identified for FGFR3 and FGFR4. FGF receptors with alternative IgIII domains, referred to herein as “FGFRIIIb” and “FGFR2IIIc,” are generated by splicing events of FGFR1-3 involving the C-terminal half of the IgIII domain encoded by two mutually exclusive alternative exons derived from the FGFR2 gene (reviewed in Galzie, Z. et al. (1997) Biochem. Cell. Biol. 75:669-685; Burke, D. et al. (1998) Trends Biochem Sci 23:59-62). FGFR2-IIIc uses the alternative exon III, which encodes a different sequence than that of isoform FGFR2-IIIb. Other causes/sources of alternative splicing include frameshifting (i.e., different set of triplet codons in the mRNA/transcript is translated by the ribosome) or varying translation start or stop site (on the mRNA during its translation), resulting in a given intron remaining in the mRNA transcript. Different body tissues and some diseases are associated with alternative splicing events, and thus result in different proteins being produced in different tissues; or in diseased tissues.

An “oncogenic isoform” refers to any protein, polypeptide, mRNA, or cDNA that can be derived from one or more of alternative splicing, frameshifting, translational and/or post-translational events, whose presence or abnormal level is associated with cancer or malignant phenotype. For example, it may be found at an abnormal level in cells derived from disease-affected tissues, as compared to a reference value, e.g., a tissue or cells of a non disease control. It may be a protein isoform that is expressed at an abnormally high level, where the altered expression correlates with the occurrence and/or progression of the cancer. An oncogenic isoform may also be the expression product of a gene possessing mutation(s) or genetic variation that is directly responsible or is in linkage disequilibrium with other gene(s) that are responsible for the etiology of cancer. Exemplary oncogenic isoforms include, but are not limited to, FGFR2 (e.g., an oncogenic FGFR2 isoform IIIc), FGFR1 (e.g., an oncogenic FGFR1L), RON receptor tyrosine kinase (e.g., an oncogenic RON receptor tyrosine kinase comprising a deletion of exons 5 and 6), KIT receptor tyrosine kinase (e.g., an oncogenic KIT receptor tyrosine kinase comprising a deletion in exon 11), and PDGF-receptor alpha (e.g., an oncogenic PDGF-receptor alpha comprising a deletion of exons 7 and 8).

Similarly, a “non-oncogenic isoform” or “non-oncogenic protooncogene” refers to a protein, polypeptide, mRNA, or cDNA that is found predominantly in non-cancerous cells or tissues. Such isoforms and protooncogenes may be expressed in malignant conditions, but is not typically associated with the malignant phenotype.

The compositions and methods of the present invention encompass polypeptides and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., SEQ ID NO: 2, SEQ ID NO: 4 or SEQ ID NO: 6 are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., SEQ ID NO: 1, 3, or 5 are termed substantially identical.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid (SEQ ID NO: 1) molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein, the term “polypeptide” refers to two or more amino acids linked by a peptide bond between the alpha-carboxyl group of one amino acid and the alpha-amino group of the next amino acid. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a nonnatural arrangement.

An “oligonucleotide” refers to a single stranded polynucleotide having less than about 100 nucleotides, less than about 75, 50, 25, or 10 nucleotides. An “oligonucleotide,” as used herein, refers to an oligomer or polymer of a ribonucleic acid (RNA) or deoxyribonucleic acid (DNA) or mimetics thereof. This term includes oligonucleotides composed of naturally occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

Various aspects of the invention are described in further detail below. Additional definitions are set out throughout the specification.

### Polypeptides of Oncogenic Isoforms, or Epitopes Thereof

The invention provides isolated polypeptides of oncogenic isoforms or epitope thereof, or substantially identical sequences thereto. The term “epitope” or “epitope fragment” refers to the region of an antigen to which an antibody molecule binds preferentially and specifically. A monoclonal antibody binds preferentially to a single specific epitope of a molecule that can be molecularly defined. An epitope of a particular protein or protein isoform may be constituted by a limited number of amino acid residues, e.g. 2-30 residues, that are either in a linear or non-linear organization on the protein or protein isoform. An epitope that is recognized by the antibody may be, e.g., a short peptide of 2-30 amino acids that spans a junction of two domains or two polypeptide fragments of an oncogenic isoform that is not present in the normal isoforms of the protein. An oncogenic isoform may be a translation product of an alternatively spliced RNA variant that either lacks one or more exon(s) or has additional exon(s) relative to the RNA encoding the normal protein. The epitope may comprise, or consist of, residues at positions 15-16, 15-17, 15-18, 15-19, 15-20, 15-21, 15-22, 15-23, 15-24, 15-25, 15-26, 15-27, 15-28, 15-29, or 15-30 of any one of SEQ ID NOs: 10, 12, 14, or 18. In another embodiment, the epitope may comprise, or consist of, residues at positions 14-16, 14-17, 14-18, 14-19, 14-20, 14-21, 14-22, 14-23, 14-24, 14-25, 14-26, 14-27, 14-28, 14-29, or 14-30 of any one of SEQ ID NOs: 10, 12, 14, or 18. In another embodiment, the epitope may comprise, or consist of, residues at positions 13-16, 13-17, 13-18, 13-19, 13-20, 13-21, 13-22, 13-23, 13-24, 13-25, 13-26, 13-27, 13-28, 13-29, or 13-30 of any one of SEQ ID NOs: 10, 12, 14, or 18. In another embodiment, the epitope may comprise, or consist of, residues at positions 12-16, 12-17, 12-18, 12-19, 12-20, 12-21, 12-22, 12-23, 12-24, 12-25, 12-26, 12-27, 12-28, 12-29, or 12-30 of any one of SEQ ID NOs: 10, 12, 14, or 18. In another embodiment, the epitope may comprise, or consist of, residues at positions 11-16, 11-17, 11-18, 11-19, 11-20, 11-21, 11-22, 11-23, 11-24, 11-25, 11-26, 11-27, 11-28, 11-29, or 11-30 of any one of SEQ ID NOs: 10, 12, 14, or 18. In another embodiment, the epitope may comprise, or consist of, residues at positions 10-16, 10-17, 10-18, 10-19, 10-20, 10-21, 10-22, 10-23, 10-24, 10-25, 10-26, 10-27, 10-28, 10-29, or 10-30 of any one of SEQ ID NOs: 10, 12, 14, or 18. In another embodiment, the epitope may comprise, or consist of, residues at positions 9-16, 9-17, 9-18, 9-19, 9-20, 9-21, 9-22, 9-23, 9-24, 9-25, 9-26, 9-27, 9-28, 9-29, or 9-30 of any one of SEQ ID NOs: 10, 12, 14, or 18. In another embodiment, the epitope may comprise, or consist of, residues at positions 8-16, 8-17, 8-18, 8-19, 8-20, 8-21, 8-22, 8-23, 8-24, 8-25, 8-26, 8-27, 8-28, 8-29, or 8-30 of any one of SEQ ID NOs: 10, 12, 14, or 18. In another embodiment, the epitope may comprise, or consist of, residues at positions 7-16, 7-17, 7-18, 7-19, 7-20, 7-21, 7-22, 7-23, 7-24, 7-25, 7-26, 7-27, 7-28, 7-29, or 7-30 of any one of SEQ ID NOs: 10, 12, 14, or 18. In another embodiment, the epitope may comprise, or consist of, residues at positions 6-16, 6-17, 6-18, 6-19, 6-20, 6-21, 6-22, 6-23, 6-24, 6-25, 6-26, 6-27, 6-28, 6-29, or 6-30 of any one of SEQ ID NOs: 10, 12, 14, or 18. In another embodiment, the epitope may comprise, or consist of, residues at positions 5-16, 5-17, 5-18, 5-19, 5-20, 5-21, 5-22, 5-23, 5-24, 5-25, 5-26, 5-27, 5-28, 5-29, or 5-30 of any one of SEQ ID NOs: 10, 12, 14, or 18. In another embodiment, the epitope may comprise, or consist of, residues at positions 4-16, 4-17, 4-18, 4-19, 4-20, 4-21, 4-22, 4-23, 4-24, 4-25, 4-26, 4-27, 4-28, 4-29, or 4-30 of any one of SEQ ID NOs: 10, 12, 14, or 18. In another embodiment, the epitope may comprise, or consist of, residues at positions 3-16, 3-17, 3-18, 3-19, 3-20, 3-21, 3-22, 3-23, 3-24, 3-25, 3-26, 3-27, 3-28, 3-29, or 3-30 of any one of SEQ ID NOs: 10, 12, 14, or 18. In another embodiment, the epitope may comprise, or consist of, residues at positions 2-16, 2-17, 2-18, 2-19, 2-20, 2-21, 2-22, 2-23, 2-24, 2-25, 2-26, 2-27, 2-28, 2-29, or 2-30 of any one of SEQ ID NOs: 10, 12, 14, or 18. In another embodiment, the epitope may comprise, or consist of, residues at positions 1-16, 1-17, 1-18, 1-19, 1-20, 1-21, 1-22, 1-23, 1-24, 1-25, 1-26, 1-27, 1-28, 1-29, or 1-30 of any one of SEQ ID NOs: 10, 12, 14, or 18. The “epitope” may be used to raise antibodies that specifically bind the oncogenic isoform (e.g., do not substantially bind to the non-oncogenic isoform derived from the same proto-oncogene).

In one embodiment, the invention provides isolated polypeptides of human oncogenic isoforms or epitope thereof. In one embodiment, an isoform or epitope thereof is an oncogenic form of a proto-oncogene selected from the group consisting of human FGFR2 (SEQ ID NO: 32), human FGFR1 (SEQ ID NO: 33), human RON Receptor tyrosine kinase (SEQ ID NO: 34), human KIT receptor tyrosine kinase (SEQ ID NO: 35), human PDGF (SEQ ID NO: 36), and human PDGFR-alpha (SEQ ID NO: 37), or a sequence substantially identical thereto.

In one embodiment, the invention provides isolated rat polypeptides of oncogenic isoforms or epitope thereof. In one embodiment, the invention provides isolated mouse polypeptides of human oncogenic isoforms or epitope thereof. In other embodiments, the isolated polypeptides of human oncogenic isoforms or epitope thereof will be derived from other species, including but not limited to, dogs, pigs, guinea pigs and rabbits.

### FGFR2

Fibroblast growth factor receptor 2 (FGFR2), also known in the art as bacteria-expressed kinase (BEK), keratinocyte growth factor receptor (KGFR), JWS, CEK3, CFD1, ECT1, TK14, TK25, BFR-1, CD332, K-SAM and FLJ98662. FGFR2 is a member of the fibroblast growth factor receptor family and has high affinity for acidic, basic and/or keratinocyte growth factor. FGFR2 is associated with signal transduction leading to mitogenesis and differentiation. Mutations in FGFR2 have been associated with craniofacial dysostosis 1, Crouzon syndrome, Pfeiffer syndrome, Jackson-Weiss syndrome and Apert syndrome.

The nucleotide acid and protein sequences of human FGFR2 are disclosed, e.g., in Dionne et al., (1990) EMBO J. 9:2685-2692 and Mild et al., (1992) PNAS 89:246-250). The nucleotide and protein sequences of mouse FGFR2 are disclosed, e.g., in Mild et al., (1991) Science 251:72-75 and Mansukhani et al., (1992) PNAS 89:3305-3309. The unprocessed precursor of human FGFR2 is about 821 amino acids in length and about 90310 Da in molecular weight. The unprocessed precursor of mouse FGFR2 is about 821 amino acids in length and about 90310 Da in molecular weight.

In one embodiment, the invention provides isolated polypeptides of oncogenic isoforms or epitope thereof encoded by a nucleic acid comprising a segment of nucleotides which arise from an alternative use of Exon III of a nucleic acid encoding a FGFR2. In one embodiment, the alternative use of Exon III results in sequence variation in the region of amino acids from 301-360, when aligned with FGFR2 IIIb. Thus, in one embodiment, the polypeptide consists of, or comprises, a sequence selected from the group of SEQ NOs: 2, 4, 6, and 8. In another embodiment, the polypeptide consists of, or comprises, a sequence encoded by a nucleic acid selected from the group consisting of SEQ NOs: 1, 3, 5, and 7, or sequences substantially identical to the same.

### FGFR1

Fibroblast growth factor receptor 1 (FGFR1) is also known in the art as CEK; FLG; FLT2; KAL2; BFGFR; CD331; FGFBR; HBGFR; N-SAM and FLJ99988. FGFR1 is a member of the fibroblast growth factor receptor family and has high affinity for both acidic and basic fibroblast growth factors. FGFR1 is associated with signal transduction leading to mitogenesis and differentiation and is involved in limb induction.

The nucleotide acid and protein sequences of human FGFR1 are disclosed, e.g., in Isacchi et al., Nucleic Acids Res. 18:1906-1906 (1990) and Hou et al., Science 251:665-668 (1991). The nucleotide and protein sequences of mouse FGFR1 are disclosed, e.g., in Harada et al., Biochem. Biophys. Res. Commun. 205:1057-1063 (1994). The unprocessed precursor of human FGFR1 is about 822 amino acids in length and about 90420 Da in molecular weight. The unprocessed precursor of mouse FGFR1 is about 822 amino acids in length and about 90420 Da in molecular weight.

Mutations in FGFR1 have been associated with Pfeiffer syndrome, Jackson-Weiss syndrome, Antley-Bixler syndrome, osteoglophonic dysplasia, and autosomal dominant Kallmann syndrome 2. Chromosomal aberrations involving this gene are associated with stem cell myeloproliferative disorder and stem cell leukemia lymphoma syndrome.

In one embodiment, the invention provides isolated polypeptides of oncogenic isoforms or epitope thereof encoded by a nucleic acid comprising a segment of nucleotides which arise from an alternative deletion of Exons 7 and 8 of a nucleic acid encoding a FGFR1. In one embodiment, the alternative deletion of Exons 7 and 8 results in a deletion of 105 amino acids, when aligned with an FGFR1 proto-oncogene. Thus, in one embodiment, the polypeptide consists of, or comprises, a sequence of SEQ NO: 10, or a sequence substantially identical to the same. In another aspect the polypeptide comprises a sequence encoded by a nucleic acid sequence of SEQ NO: 9, or a sequence substantially identical to the same.

### RON Receptor Tyrosine Kinase

Macrophage stimulating 1 receptor (c-met-related tyrosine kinase) (RON) is also known in the art as MST1R, PTK8, CD136 and CDw136. RON is a receptor for macrophage stimulating protein (MSP) and has a tyrosine-protein kinase activity. It is involved in development of epithelial tissue, bone and neuroendocrine derivatives. The nucleotide acid and protein sequences of human RON are disclosed, e.g., in Ronsin C. et al., Oncogene 8:1195-1202 (1993); and Collesi C. et al., Mol. Cell. Biol. 16:5518-5526 (1996). The nucleotide acid and protein sequences of mouse RON are disclosed e.g., in Iwama A. et al., Blood 83:3160-3169 (1994); Waltz S. E. et al., Oncogene 16:27-42 (1998); and Persons D. A. et al., Nat. Genet. 23:159-165 (1999). The unprocessed precursor of human RON is about 1400 amino acids in length and about 152227 Da in molecular weight. The unprocessed precursor of mouse RON is about 1378 amino acids in length and about 150538 Da in molecular weight.

In one embodiment, the invention provides isolated polypeptides of oncogenic isoforms or epitope fragments thereof encoded by a nucleic acid comprising a segment of nucleotides which arise from an alternative deletion of Exons 5 and 6 of a nucleic acid encoding a RON receptor tyrosine kinase. In one embodiment, the alternative deletion of Exons 5 and 6 results in an in-frame deletion of 109 amino acids in the extracellular domain, when aligned with a RON receptor tyrosine kinase proto-oncogene. In one embodiment, the polypeptide consists of, or comprises, a polypeptide sequence resulting from the fusion and juxtaposition of Exons 4 and 7. Thus, in one embodiment, the polypeptide consists of, or comprises, a sequence of SEQ NO: 12, or a sequence substantially identical to the same. In another embodiment, the polypeptide consists of, or comprises, a sequence encoded by a nucleic acid sequence of SEQ NO: 11, or a sequence substantially identical to the same.

### KIT Receptor Tyrosine Kinase

v-kit Hardy-Zuckerman 4 feline sarcoma viral oncogene homolog (KIT) is also known in the art as PBT; SCFR; C-Kit and CD117. KIT encodes the human homolog of the proto-oncogene c-kit. KIT is a type 3 transmembrane receptor for MGF (mast cell growth factor, also known as stem cell factor).

The nucleotide acid and protein sequences of human KIT are disclosed, e.g., in Yarden et al., EMBO J. 6:3341-3351 (1987) and Giebel et al., Oncogene 7:2207-2217 (1992). The nucleotide acid and protein sequences of mouse KIT are disclosed e.g., in. Qiu et al., EMBO J. 7:1003-1011 (1988) and Rossi et al., Dev. Biol. 152:203-207 (1992). The unprocessed precursor of human KIT is about 976 amino acids in length and about 107360 Da in molecular weight. The unprocessed precursor of mouse KIT is about 107250 amino acids in length and about 150538 Da in molecular weight.

Mutations in KIT are associated with gastrointestinal stromal tumors, mast cell disease, acute myelogenous leukemia, and piebaldism.

In one embodiment, the invention provides isolated polypeptides of oncogenic isoforms or epitope fragments thereof encoded by a nucleic acid comprising a segment of nucleotides which arise from an alternative deletion of Exon 11 of a nucleic acid encoding a KIT receptor tyrosine kinase. Thus, in one embodiment, the polypeptide consists of, or comprises, a sequence of SEQ NO: 14, or a sequence substantially identical to the same. In another embodiment, the polypeptide consists of, or comprises, a sequence encoded by a nucleic acid sequence of SEQ NO: 13, or a sequence substantially identical to the same.

### PDGF

Platelet-derived growth factor alpha polypeptide (PDGFA) is also known in the art as PDGF1 and PDGF-A. PDGFA encoded a member of the platelet-derived growth factor family. PDGFA is a mitogenic factor for cells of mesenchymal origin and is characterized by a motif of eight cysteines.

The nucleotide acid and protein sequences of human PDGFA are disclosed, e.g., in Bonthron et al., Proc. Natl. Acad. Sci. U.S.A. 85:1492-1496 (1988) and Betsholtz et al., Nature 320:695-699 (1986). The nucleotide acid and protein sequences of mouse PDGFA are disclosed e.g., in. Rorsman et al., Growth Factors 6:303-313 (1992) and Mercola et al., Dev. Biol. 138:114-122 (1990). The unprocessed precursor of human PDGFA is about 211 amino acids in length and about 23210 Da in molecular weight. The unprocessed precursor of mouse PDGFA is about 211 amino acids in length and about 23210 Da in molecular weight.

Studies using knockout mice have shown cellular defects in oligodendrocytes, alveolar smooth muscle cells, and Leydig cells in the testis; knockout mice die either as embryos or shortly after birth.

In one embodiment, the invention provides isolated polypeptides of oncogenic isoforms or epitope fragments thereof encoded by a nucleic acid comprising a segment of nucleotides which arise from an alternative in-frame deletion of Exon 6 of a nucleic acid encoding PDGF. Thus, in one embodiment, the polypeptide consists of, or comprises, a sequence of SEQ NO: 16, or sequence substantially identical to the same. In another embodiment, the polypeptide consists of, or comprises, a sequence encoded by a nucleic acid sequence of SEQ NO: 15, or a sequence substantially identical to the same.

### PDGFR-alpha

Platelet-derived growth factor receptor, alpha polypeptide (PFGFRA) is also known in the art as CD140A; PDGFR2; MGC74795 and Rhe-PDGFRA. PFGFRA encodes a cell surface tyrosine kinase receptor for members of the platelet-derived growth factor family. These growth factors are mitogens for cells of mesenchymal origin.

The nucleotide acid and protein sequences of human PDGFA are disclosed e.g., in Bonthron et al., Proc. Natl. Acad. Sci. U.S.A. 85:1492-1496 (1988) and Betsholtz et al., Nature 320:695-699 (1986). The nucleotide acid and protein sequences of mouse PDGFA are disclosed, e.g., in Stiles et al., Mol. Cell. Biol. 10:6781-6784 (1990) and Carninci et al., Science 309:1559-1563 (2005). The unprocessed precursor of human PDGFA is about 1089 amino acids in length and about 119790 Da in molecular weight. The unprocessed precursor of mouse PDGFA is about 1089 amino acids in length and about 119790 Da in molecular weight.

A fusion of PDGFRA and FIP1L1 (FIP1L1-PDGFRA), due to an interstitial chromosomal deletion, is the cause of some cases of hypereosinophilic syndrome (HES). HES is a rare hematologic disorder characterized by sustained overproduction of eosinophils in the bone marrow, eosinophilia, tissue infiltration and organ damage.

In one embodiment, the invention provides isolated polypeptides of oncogenic isoforms or epitopes thereof encoded by a nucleic acid comprising a segment of nucleotides which arise from an alternative deletion of Exons 7 and 8 (e.g., amino acids 374-456) of a nucleic acid encoding PDGFR-alpha. Thus, in one embodiment, the polypeptide consists of, or comprises, a sequence of SEQ NO: 18, or a sequence substantially identical to the same. In another embodiment, the polypeptide consists of, or comprises, a sequence encoded by a nucleic acid sequence of SEQ NO: 17, or a sequence substantially identical to the same.

Alternatively, an isolated polypeptide of an oncogenic isoform or epitope thereof may be encoded by a nucleic acid which is substantially identical to a nucleic acid of an oncogenic isoform or epitope fragment thereof provided herein. Likewise, an isolated polypeptide of an oncogenic isoform or epitope thereof may be substantially identical to an oncogenic isoform or epitope thereof, as provided herein.

### Methods of Preparing an Oncogenic Isoform or Epitope Fragment Thereof

The polypeptide oncogenic isoform or epitope fragment thereof can be isolated from natural sources, or can be a product of chemical synthetic procedures, or can be produced by recombinant techniques from a prokaryotic or eukaryotic host.

The invention also provides methods of preparing an oncogenic isoform or epitope fragment thereof, comprising culturing host cells under conditions that permit expression of the oncogenic isoform or epitope fragment thereof; and isolating the oncogenic isoform or epitope fragment thereof, thereby preparing the oncogenic isoform or epitope fragment thereof. In one embodiment, the invention provides a method of preparing a human oncogenic isoform or epitope fragment thereof. Procedures for preparing a polypeptide using the above describe method are well known to those skilled in the art.

### Isoform-Specific Inhibitors

The present invention provides, at least in part, isoform-specific inhibitors (e.g., antibody molecules, soluble receptor polypeptides and fusion forms thereof, peptides and functional variants thereof, and nucleic acid inhibitors), which inhibit and/or reduce one or more activities of the isoform, or interact with, or more preferably specifically bind to one or more isoform polypeptides or fragments thereof, or nucleic acids encoding one or more isoform polypeptides or fragments thereof. In one embodiment, the isoform-specific inhibitor is an isoform-binding molecule, e.g., an antibody molecule, or a nucleic acid inhibitor. In another embodiment, the isoform-specific inhibitor is a soluble receptor polypeptide and a fusion form thereof, or a peptide and a functional variant thereof. In some embodiments, the isoform-binding molecules specifically bind to oncogenic isoform polypeptides or fragments thereof, or nucleic acids encoding one or more oncogenic isoform polypeptides or fragments thereof.

Typical isoform-specific inhibitors (e.g., isoform-binding molecules) bind to one or more isoform polypeptides or fragments thereof, or nucleic acids encoding one or more isoform polypeptides or fragments thereof, with high affinity, e.g., with an affinity constant of at least about 10⁷ M⁻¹, typically about 10⁸ M⁻¹, and more typically, about 10⁹ M⁻¹ to 10¹⁰ M⁻¹ or stronger; and reduce and/or inhibit one or more activities of the isoforms, e.g., oncogenic isoforms, in a hyperproliferative (e.g., cancerous or malignant) cell and/or tissue. For example, the isoform-specific inhibitor may selectively and specifically reduce or inhibit an oncogenic isoform-associated activity chosen from one or more of: (i) binding of a ligand or co-receptor (e.g., FGF ligand (e.g., FGF8b, FGF2, FGF17 or FGF18)) to FGFR2 isoform IIIc); (ii) receptor dimerization (e.g., FGFR2 isoform IIIc homo-dimerization or FGFR2 isoform IIIc with another receptor or receptor isoform hetero-dimerization); (iii) isoform signaling, e.g., FGFR2 isoform IIIc signaling; (iv) hyperproliferative (e.g., cancerous or tumor) cell proliferation, growth and/or survival, for example, by induction of apoptosis of the hyperproliferative cell; and/or (v) angiogenesis and/or vascularization of a tumor.

As used herein, the term “specifically binds” refers to a binding interaction that is determinative of the presence of a target (such a specific polypeptide or nucleic acid) in a population of proteins and other biologics. Thus, a binding molecule that “specifically binds” an oncogenic isoform is intended to mean that the compound binds an oncogenic isoform of the invention, but does not bind to a non-oncogenic isoform that is derived from the same proto-oncogene. As the skilled artisan will recognize the isoform-binding molecule may show some degree of cross-reactivity between the oncogenic and non-oncogenic isoforms depending on the conditions used, e.g., target protein concentration, salt and buffer conditions used, among others. In certain embodiments, the term “specifically binds” or “specific binding” refers to a property of the isoform-binding molecule to bind to one or more isoform polypeptides or fragments thereof, or nucleic acids encoding one or more isoform polypeptides or fragments thereof, with high affinity, e.g., with an affinity constant of at least about 10⁷ M⁻¹, typically about 10⁸ M⁻¹, and more typically, about 10⁹ M⁻¹ to 10¹⁰ M⁻¹ or stronger, and (2) preferentially bind to the isoform with an affinity that is at least two-fold, 50-fold, 100-fold, 1000-fold, or more greater than its affinity for binding to the non-oncogenic isoform. In certain embodiments, isoform-binding molecule binds preferentially to an oncogenic isoform, but does not substantially bind to (e.g., shows less than 10%, 8%, 5%, 4%, 3%, 2%, 1% cross-reactivity with) to its non-oncogenic counterpart.

### Antibody Molecules

In one embodiment, the isoform-binding molecule is an antibody molecule that binds to a mammalian, e.g., human, isoform polypeptide or a fragment thereof (e.g., an Fab, F(ab′)₂, Fv, a single chain Fv fragment, or a camelid variant). For example, the antibody molecule binds to an isoform polypeptide or fragment expressed and/or associated with a hyperproliferative cell, e.g., a cancerous or tumor cell. For example, the antibody molecule binds specifically to an epitope, e.g., linear or conformational epitope, (e.g., an epitope as described herein) located or expressed primarily on the surface of a hyperproliferative cell, e.g., a cancerous or tumor cell. In embodiments, the epitope recognized by the antibody molecule is expressed or associated with a hyperproliferative disease, e.g., a cancerous or malignant disease. For example, the epitope recognized by the antibody molecule is expressed or associated with an exon sequence predominantly expressed or associated with one or more cancerous or tumor cells or disorders; the epitope may be located at the junctional region between two exons that are predominantly joined together in one or more cancerous or tumor cells or disorders, e.g., as a result of an in-frame exon deletion or the use of an alternatively spliced exon. Exemplary isoform polypeptides or fragments recognized by isoform-binding molecules of the invention include, but are not limited to, oncogenic isoforms of FGFR2, FGFR1, RON receptor tyrosine kinase, KIT receptor tyrosine kinase, PDGF and PDGF-receptor alpha. In one embodiment, the oncogenic isoform to which the antibody molecule binds is a human oncogenic isoform. In another embodiment, the polypeptide isoform to which the antibody molecule binds is a polypeptide of an oncogenic isoform or epitope thereof listed in Table 1.

In one embodiment, the antibody molecule specifically binds a polypeptide comprising the amino acid sequence set forth in SEQ ID NO: 2, 4, 6, 8, 10, 12, 14, 16, or 18, or a substantially identical sequence thereto. In another embodiment, the antibody molecule specifically binds to the polypeptide FGFR2-IIIc isoform of SEQ ID NO: 2, 4, 6, or 8, but does not substantially bind to the polypeptide isoform of human FGFR2-IIIb. In another embodiment, the antibody molecule binds to the human FGFR2 polypeptide of e.g., SEQ ID NO: 19, but does not substantially bind to FGFR2-IIIb (e.g., SEQ ID NO: 21) or other isoforms of FGFR2 (e.g., SEQ ID NOs: 22, 23, 24, 25, 26, 27, 28, 29, 30, 31, 52 and/or 53, respectively).

In yet another embodiment, the antibody molecule specifically binds to the polypeptide FGFR1L isoform of SEQ ID NO:10, but does not substantially bind to full-length FGFR1 or other non-oncogenic polypeptide FGFR1 isoforms (Isoform-1: SEQ ID NO: 38; Isoform-4: SEQ ID NO: 39; Isoform-14: SEQ ID NO: 40; Isoform-16: SEQ ID NO: 41; Isoform-17: SEQ ID NO: 42; Isoform-3: SEQ ID NO: 43; or Isoform-18: SEQ ID NO: 44, respectively).

In yet another embodiment, the antibody molecule specifically binds to the human polypeptide RON receptor tyrosine kinase Δ160 isoform, but does not substantially bind to other non-oncogenic polypeptide isoforms of RON receptor tyrosine kinase (e.g., SEQ ID NO: 45). In another embodiment, the antibody molecule specifically binds to the junction between exons 4 and 7 of this A160 isoform. In another embodiment, the antibody molecule specifically binds to the polypeptide of SEQ ID NO: 12 or a substantially identical sequence thereto.

In one embodiment, the antibody molecule specifically binds to the human polypeptide KIT receptor tyrosine kinase isoform with a deletion in exon 11, but does not substantially bind to another polypeptide isoform without the deletion. In one embodiment, the antibody molecule specifically binds to the polypeptide KIT receptor tyrosine kinase isoform as set forth in SEQ ID NO: 46, but does not substantially bind to other polypeptide isoforms of KIT receptor tyrosine kinase (e.g., full-length receptor; SEQ ID NO: 47). In one embodiment, the antibody molecule specifically binds to the junction at the deletion in exon 11 of this oncogenic isoform. In another embodiment, the antibody molecule specifically binds to the polypeptide of SEQ ID NO: 14 or a substantially identical sequence thereof.

In one embodiment, the antibody molecule specifically binds to the human polypeptide PDGF isoform 2 with an in-frame deletion of exon 6, but does not substantially bind to another PDGF isoform without the deletion. In one embodiment, the antibody molecule specifically binds to the PDGF isoform 2 as set forth in SEQ ID NO: 48, but does not substantially bind to other polypeptide isoforms of PDGF (e.g., full-length PDGF (SEQ ID NO: 49). In one embodiment, the antibody molecule specifically binds to the junction at the deletion of exon 6 in this oncogenic isoform. In another embodiment, the antibody molecule specifically binds to the polypeptide of SEQ ID NO: 16 or a substantially identical sequence thereof.

In one embodiment, the antibody molecule specifically binds to the human polypeptide PDGFR-alpha isoform (SEQ ID NO: 51) with a deletion of exons 7 and 8, but does not substantially bind to another PDGFR isoform without the deletion. In one embodiment, the antibody molecule specifically binds to the PDGFR-alpha isoform, but does not substantially bind to other polypeptide isoforms of PDGFR (such as isoform-1 (SEQ ID NO: 50)). In one embodiment, the antibody molecule specifically binds to the junction at the deletion of exons 7 and 8 in this oncogenic isoform. In another embodiment, the antibody molecule specifically binds to the polypeptide of SEQ ID NO: 18 or a substantially identical sequence thereof.

As used herein, the term “antibody molecule” refers to a protein comprising at least one immunoglobulin variable domain sequence. The term antibody molecule includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibodies of the present invention can be monoclonal or polyclonal. The antibody can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda.

Examples of antigen-binding fragments include: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

Antibodies of the present invention can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR). The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modelling software. See, generally, e.g., Protein Sequence and Structure Analysis of Antibody Variable Domains. In: Antibody Engineering Lab Manual (Ed.: Duebel, S, and Kontermann, R., Springer-Verlag, Heidelberg). Generally, unless specifically indicated, the following definitions are used: AbM definition of CDR1 of the heavy chain variable domain and Kabat definitions for the other CDRs. In addition, embodiments of the invention described with respect to Kabat or AbM CDRs may also be implemented using Chothia hypervariable loops. Each VH and VL typically includes three CDRs and four FRs, arranged from amino-terminus to carboxy-terminus in the following order: FR1, CDR1, FR2, CDR2, FR3, CDR3, FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to the isoform polypeptide, or an epitope thereof. With respect to proteins (or protein mimetics), the antigen-binding site typically includes one or more loops (of at least four amino acids or amino acid mimics) that form an interface that binds to the isoform polypeptide. Typically, the antigen-binding site of an antibody molecule includes at least one or two CDRs, or more typically at least three, four, five or six CDRs.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

The anti-isoform antibody can be a polyclonal or a monoclonal antibody. In other embodiments, the antibody can be recombinantly produced, e.g., produced by phage display or by combinatorial methods.

Phage display and combinatorial methods for generating anti-isoform antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the anti-isoform antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An anti-isoform antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to an isoform. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are known to those skilled in the art and, for example, may be obtained from a hybridoma producing an antibody against the isoform. The recombinant DNA encoding the humanized antibody, or fragment thereof, can be cloned into an appropriate expression vector.

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Preferred humanized antibodies have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In one embodiment, an antibody can be made by immunizing with purified anti-isoform antigen, or a fragment or epitope thereof, e.g., a fragment described herein, membrane associated antigen, tissue, e.g., crude tissue preparations, whole cells, preferably living cells, lysed cells, or cell fractions, e.g., membrane fractions.

The anti-isoform antibody can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target isoform protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and U.S. Pat. No. 5,648,260, the contents of all of which are hereby incorporated by reference). Similar type of alterations could be described which if applied to the murine, or other species immunoglobulin would reduce or eliminate these functions.

An isoform-specific inhibitor (e.g., an isoform-binding molecule) can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody molecule of the invention may be derivatized (or labeled) to include fluorescent compounds, various enzymes, prosthetic groups, luminescent materials, bioluminescent materials, fluorescent emitting metal atoms, e.g., europium (Eu), and other anthanides, and radioactive materials (described below). Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, β-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody molecule may also be derivatized with a prosthetic group (e.g., streptavidin/biotin and avidin/biotin). For example, an antibody may be derivatized with biotin, and detected through indirect measurement of avidin or streptavidin binding. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of bioluminescent materials include luciferase, luciferin, and aequorin.

Labeled antibody molecule can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen.

An anti-isoform antibody molecules may be conjugated to another molecular entity, typically a label or a therapeutic (e.g., a cytotoxic or cytostatic) agent or moiety.

Radioactive isotopes can be used in diagnostic or therapeutic applications. Radioactive isotopes that can be coupled to the anti-PSMA antibodies include, but are not limited to α-, β-, or γ-emitters, or β- and γ-emitters. Such radioactive isotopes include, but are not limited to iodine (¹³¹I or ¹²⁵I), yttrium (⁹⁰ Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹ mTc), phosphorus (³²P), rhodium (¹⁸⁸Rh) sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (²²⁵Ac), praseodymium, (²¹¹At), rhenium (¹⁸⁶Re), (²¹²Bi or ²¹³Bi), and rhodium (¹⁸⁸Rh). Radioisotopes astatine (²¹¹At) rhenium (¹⁸⁶Re), bismuth (²¹²Bi or ²¹³Bi) and rhodium (¹⁸⁸Rh) Radioisotopes useful as labels, e.g., for use in diagnostics, include iodine (¹³¹I or ¹²⁵I), indium (¹¹¹In), technetium (⁹⁹mTc), phosphorus (³²P), carbon (¹⁴C), and tritium (³H), or one or more of the therapeutic isotopes listed above.

The invention provides radiolabeled antibody molecules and methods of labeling the same. In one embodiment, a method of labeling an antibody molecule is disclosed. The method includes contacting an antibody molecule, with a chelating agent, to thereby produce a conjugated antibody. The conjugated antibody is radiolabeled with a radioisotope, e.g., ¹¹¹Indium, ⁹⁰Yttrium and ¹⁷⁷Lutetium, to thereby produce a labeled antibody molecule.

As is discussed above, the antibody molecule can be conjugated to a therapeutic agent. Therapeutically active radioisotopes have already been mentioned. Examples of other therapeutic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclinies (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

The conjugates of the invention can be used for modifying a given biological response. The therapeutic agent is not to be construed as limited to classical chemical therapeutic agents. For example, the therapeutic agent may be a protein or polypeptide possessing a desired biological activity. Such proteins may include, for example, a toxin such as abrin, ricin A, pseudomonas exotoxin, diphtheria toxin, or a component thereof (e.g., a component of pseudomonas exotoxin is PE38); a protein such as tumor necrosis factor, interferon, nerve growth factor, platelet derived growth factor, tissue plasminogen activator, or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors. Similarly, the therapeutic agent can be a viral particle, e.g., a recombinant viral particle, that is conjugated (e.g., via a chemical linker) or fused (e.g., via a viral coat protein) to an anti-isoform antibody of the invention.

In one aspect, the invention features a method of providing a target binding molecule that specifically binds to an isoform receptor. For example, the target binding molecule is an antibody molecule. The method includes: providing a target protein that comprises at least a portion of non-human protein, the portion being homologous to (at least 70, 75, 80, 85, 87, 90, 92, 94, 95, 96, 97, 98% identical to) a corresponding portion of a human target protein, but differing by at least one amino acid (e.g., at least one, two, three, four, five, six, seven, eight, or nine amino acids); obtaining an antibody molecule that specifically binds to the antigen; and evaluating efficacy of the binding agent in modulating activity of the target protein. The method can further include administering the binding agent (e.g., antibody molecule) or a derivative (e.g., a humanized antibody molecule) to a human subject.

This invention provides an isolated nucleic acid molecule encoding the above antibody molecule, vectors and host cells thereof. The nucleic acid molecule includes but is not limited to RNA, genomic DNA and cDNA.

### Soluble Receptors and Fusions Thereof

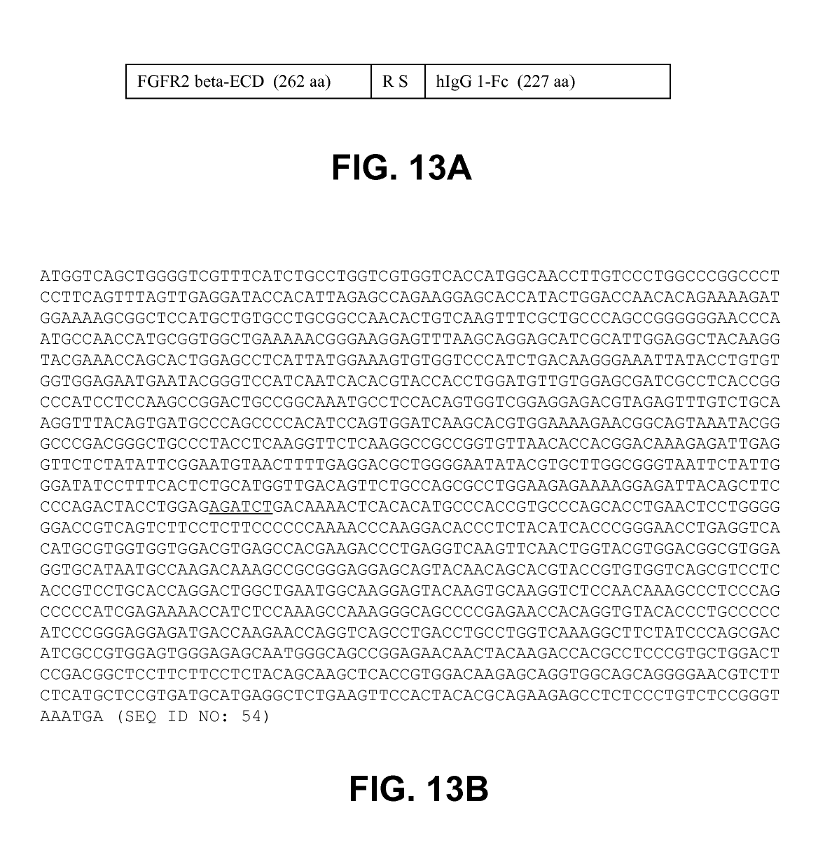

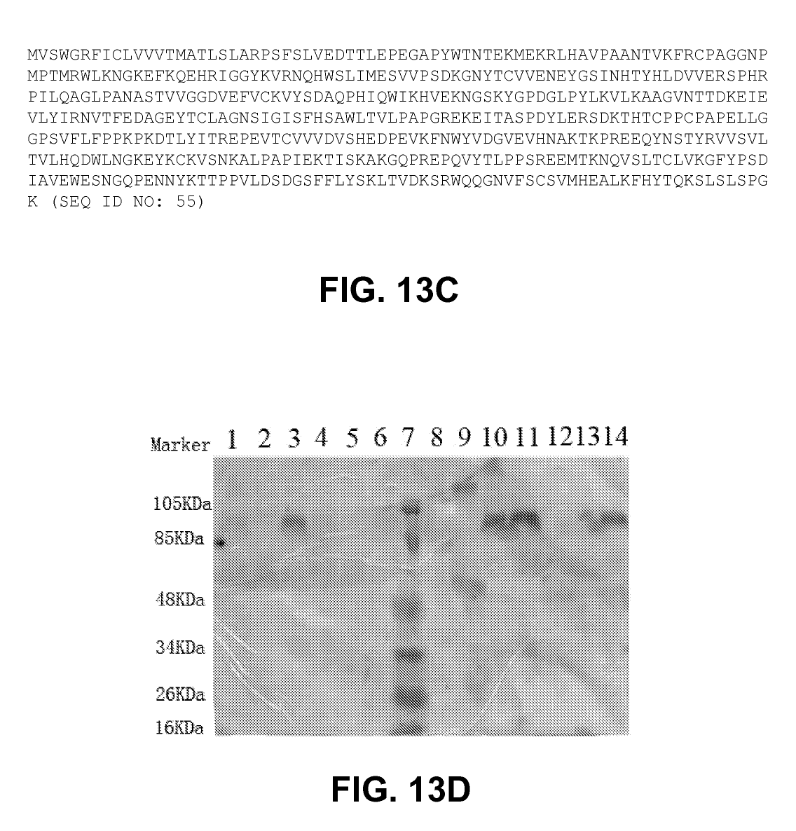

In other embodiments, the isoform-specific inhibitor is a full length or a fragment of an isoform receptor polypeptide, e.g., an inhibitory ligand-binding domain of an isoform receptor polypeptide. For example, the isoform-specific inhibitor can be a soluble form of an FGFR2 isoform IIIc receptor (e.g., a soluble form of mammalian (e.g., human) FGFR2 isoform IIIc comprising a ligand (e.g., FGF)-binding domain. For example, the isoform-specific inhibitor can include about amino acids 1 to 262 of human FGFR2 isoform IIIc receptor (FIG. 13C; amino acids 1-262 of SEQ ID NO: 55, including the signal sequence); or an amino acid sequence substantially identical thereto. Alternatively, the isoform-specific inhibitor can include an amino acid sequence encoded by the nucleotide sequence from about nucleotides 1 to 786 of human FGFR2 isoform IIIc (FIG. 13B; nucleotides 1-786 of SEQ ID NO: 54); or an amino acid sequence substantially identical thereto.

As used herein, a “soluble form of an FGFR2 isoform IIIc receptor” or a “soluble form of an isoform receptor polypeptide” is a receptor isoform, e.g., an FGFR2 isoform IIIc receptor polypeptide incapable of anchoring itself in a membrane. Such soluble polypeptides include, for example, an isoform receptor polypeptide, e.g., an FGFR2 isoform IIIc receptor polypeptide, as described herein that lack a sufficient portion of their membrane spanning domain to anchor the polypeptide or are modified such that the membrane spanning domain is non-functional. Typically, the soluble isoform receptor polypeptide retains the ability of binding to an isoform ligand, e.g., an FGF ligand. E.g., a soluble fragment of an FGFR2 isoform IIIc receptor polypeptide (e.g., a fragment of an FGFR2 isoform IIIc receptor comprising the extracellular domain of human FGFR2 isoform IIIc receptor, including about amino acids 1 to 262 of human FGFR2 isoform IIIc receptor (FIG. 13C; amino acids 1-262 of SEQ ID NO: 55, including the signal sequence); or an amino acid sequence substantially identical thereto. A soluble FGFR2 isoform IIIc receptor polypeptide can additionally include, e.g., be fused to, a second moiety, e.g., a polypeptide (e.g., an immunoglobulin chain, a GST, Lex-A or MBP polypeptide sequence). For example, a fusion protein can includes at least a fragment of an FGFR2 isoform IIIc receptor polypeptide, which is capable of binding an FGF ligand, fused to a second moiety, e.g., a polypeptide (e.g., an immunoglobulin chain, an Fc fragment, a heavy chain constant regions of the various isotypes, including: IgG1, IgG2, IgG3, IgG4, IgM, IgA1, IgA2, IgD, and IgE).

A soluble form of an isoform receptor polypeptide can be used alone or functionally linked (e.g., by chemical coupling, genetic or polypeptide fusion, non-covalent association or otherwise) to a second moiety, e.g., an immunoglobulin Fc domain, serum albumin, pegylation, a GST, Lex-A or an MBP polypeptide sequence. As used herein, a “fusion protein” refers to a protein containing two or more operably associated, e.g., linked, moieties, e.g., protein moieties. Typically, the moieties are covalently associated. The moieties can be directly associated, or connected via a spacer or linker.

The fusion proteins may additionally include a linker sequence joining the first moiety, e.g., a soluble isoform receptor, to the second moiety. For example, the fusion protein can include a peptide linker, e.g., a peptide linker of about 4 to 20, more preferably, 5 to 10, amino acids in length; the peptide linker is 8 amino acids in length. Each of the amino acids in the peptide linker is selected from the group consisting of Gly, Ser, Asn, Thr and Ala; the peptide linker includes a Gly-Ser element. In other embodiments, the fusion protein includes a peptide linker and the peptide linker includes a sequence having the formula (Ser-Gly-Gly-Gly-Gly)y wherein y is 1, 2, 3, 4, 5, 6, 7, or 8 (SEQ ID NOs: 73-80).

In other embodiments, additional amino acid sequences can be added to the N- or C-terminus of the fusion protein to facilitate expression, detection and/or isolation or purification. For example, the fusion protein may be linked to one or more additional moieties, e.g., GST, His6 tag (His-His-His-His-His-His; SEQ ID NO: 81), FLAG tag. For example, the fusion protein may additionally be linked to a GST fusion protein in which the fusion protein sequences are fused to the C-terminus of the GST (i.e., glutathione S-transferase) sequences. Such fusion proteins can facilitate the purification of the receptor fusion protein.

In another embodiment, the fusion protein is includes a heterologous signal sequence (i.e., a polypeptide sequence that is not present in a polypeptide encoded by a receptor nucleic acid) at its N-terminus. For example, the native receptor signal sequence can be removed and replaced with a signal sequence from another protein. In certain host cells (e.g., mammalian host cells), expression and/or secretion of receptor can be increased through use of a heterologous signal sequence.

A chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) Current Protocols in Molecular Biology, John Wiley & Sons, 1992). Moreover, many expression vectors are commercially available that encode a fusion moiety (e.g., an Fc region of an immunoglobulin heavy chain). A receptor encoding nucleic acid can be cloned into such an expression vector such that the fusion moiety is linked in-frame to the immunoglobulin protein.

In some embodiments, receptor fusion polypeptides exist as oligomers, such as dimers or trimers.

In other embodiments, the receptor polypeptide moiety is provided as a variant receptor polypeptide having a mutation in the naturally-occurring receptor sequence (wild type) that results in higher affinity (relative to the non-mutated sequence) binding of the receptor polypeptide to a corresponding ligand.

In other embodiments, additional amino acid sequences can be added to the N- or C-terminus of the fusion protein to facilitate expression, steric flexibility, detection and/or isolation or purification. The second polypeptide is preferably soluble. In some embodiments, the second polypeptide enhances the half-life, (e.g., the serum half-life) of the linked polypeptide. In some embodiments, the second polypeptide includes a sequence that facilitates association of the fusion polypeptide with a second polypeptide. In embodiments, the second polypeptide includes at least a region of an immunoglobulin polypeptide. Immunoglobulin fusion polypeptides are known in the art and are described in e.g., U.S. Pat. Nos. 5,516,964; 5,225,538; 5,428,130; 5,514,582; 5,714,147; and 5,455,165. For example, a soluble form of a receptor can be fused to a heavy chain constant region of the various isotypes, including: IgG1, IgG2, IgG3, IgG4, IgM, IgA1, IgA2, IgD, and IgE). Typically, the fusion protein can include the extracellular domain of a human receptor (or a sequence homologous thereto), and, e.g., fused to, a human immunoglobulin Fc chain, e.g., human IgG (e.g., human IgG1 or human IgG2, or a mutated form thereof).

The Fc sequence can be mutated at one or more amino acids to reduce effector cell function, Fc receptor binding and/or complement activity. Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and U.S. Pat. No. 5,648,260). Similar type of alterations could be described which if applied to the murine, or other species immunoglobulin would increase or decrease these functions. For example, it is possible to alter the affinity of an Fc region of an antibody (e.g., an IgG, such as a human IgG) for an FcR (e.g., Fc gamma R1), or for C1q binding by replacing the specified residue(s) with a residue(s) having an appropriate functionality on its side chain, or by introducing a charged functional group, such as glutamate or aspartate, or perhaps an aromatic non-polar residue such as phenylalanine, tyrosine, tryptophan or alanine (see e.g., U.S. Pat. No. 5,624,821).

In embodiments, the second polypeptide has less effector function that the effector function of a Fc region of a wild-type immunoglobulin heavy chain. Fc effector function includes for example, Fc receptor binding, complement fixation and T cell depleting activity (see for example, U.S. Pat. No. 6,136,310). Methods for assaying T cell depleting activity, Fc effector function, and antibody stability are known in the art. In one embodiment, the second polypeptide has low or no detectable affinity for the Fc receptor. In an alternative embodiment, the second polypeptide has low or no detectable affinity for complement protein C1q. In other embodiments, the second polypeptide has increased effector cell function, e.g., increased binding to an Fc receptor (e.g., FcγRI, FcγRIIA, FcγRIIB, FcγRIIIA and FcRn receptors) as described in, for example, Shields et al. (JBC, 276:6591-6604, 2001) and U.S. Pat. No. 6,737,056.

It will be understood that the antibody molecules and soluble receptor or fusion proteins described herein can be functionally linked (e.g., by chemical coupling, genetic fusion, non-covalent association or otherwise) to one or more other molecular entities, such as an antibody (e.g., a bispecific or a multispecific antibody), toxins, radioisotopes, cytotoxic or cytostatic agents, among others.

### Peptides or Functional Variants Thereof

In yet another embodiment, the isoform-specific inhibitor includes a peptide or a functional variant thereof (e.g., a functional analog or derivative thereof). As used herein, an “analog” of a peptide refers to a compound wherein the amino acid sequence of the compound is the same as that of the peptide except for up to 10, typically up to 8, up to 6, up to 5, up to 4, up to 3, up to 2, or up to 1 amino acid insertions, deletions, and/or substitutions of the amino acid sequence of the peptide. Typically, an analog binds to the same biological receptor as the peptide and thus displays at least some of the biological activity of the peptide. The peptide may be “derivatized” or linked to another functional molecule (e.g., another peptide or protein, e.g., a carrier protein), and/or by the addition of a fluorescent moiety, a radionucleotide, a toxin, an enzyme, polyethylene glycol (PEG), or an affinity ligand such as biotin.

As used herein, the term “carrier protein” is a protein or peptide that improves the production of antibodies to a protein to which it is associated and/or can be used to detect a protein with which it is associated. Many different carrier proteins can be used for coupling with peptides for immunization purposes. The choice of which carrier to use should be based on immunogenicity, solubility, whether adequate conjugation with the carrier can be achieved and screening assays used to identify antibodies to target proteins. The two most commonly used carriers are keyhole limpet hemocyanin (KLH) and bovine serum albumin (BSA). Other examples include secretory alkaline phosphatase (SEAP), horseradish peroxidase, luciferase, beta-galactosidase, IgG Fc (gamma chain), Glutathione-S-Transferase (GST), polyhistidine containing tags and other enzymes like beta-lactamase, other secretary proteins or peptides.

A modified peptide, conjugate or compound of the invention comprises a reactive group covalently attached to the peptide or protein. The reactive group is chosen for its ability to form a stable covalent bond with a serum protein or peptide, for example, by reacting with one or more amino groups, hydroxyl groups, or thiol groups on the serum protein or peptide. Typically, a reactive group reacts with only one amino group, hydroxyl group, or thiol group on the serum protein or peptide. Typically, a reactive group reacts with a specific amino group, hydroxyl group, or thiol group on the serum protein or peptide. A conjugate of the invention comprises a modified peptide, which is covalently attached to a serum protein or peptide via a reaction of the reactive group with an amino group, hydroxyl group, or thiol group on the serum protein or peptide. Thus, a conjugate of the invention comprises a modified peptide, in which a residue of the reactive group has formed a covalent bond to a serum protein or peptide. As used herein, “a residue of a reactive group” or “a reactive group residue” refers to the chemical structure resulting from covalent bond formation between the reactive group and another moiety, e.g., a peptide or protein present in blood. In embodiments of the modified peptides, conjugates or compounds of the invention, the reactive group is a maleimide containing group selected from gamma-maleimide-butrylamide (GMBA), maleimido propionic acid (MPA), N-hydroxysuccinimide (NHS), N-hydroxy-sulfosuccinimide (sulfo-NHS), maleimide-benzoyl-succinimide (MB S) and gamma-maleimido-butyryloxy succinimide ester (GMBS).

The peptides of the invention, including peptide linker groups, may be synthesized by standard methods of solid or solution phase peptide chemistry. A summary of the solid phase techniques may be found in Stewart and Young (1963) Solid Phase Peptide Synthesis, W. H. Freeman Co. (San Francisco), and Meienhofer (1973) Hormonal Proteins and Peptides, Academic Press (New York). For classical solution synthesis see Schroder and Lupke, The Peptides, Vol. 1, Academic Press (New York).

In general, these methods comprise the sequential addition of one or more amino acids or suitably protected amino acids to a growing peptide chain. Normally, either the amino or carboxyl group of the first amino acid is protected by a suitable protecting group. The protected amino acid is then either attached to an inert solid support or utilized in solution by adding the next amino acid in the sequence having the complimentary (amino or carboxyl) group suitably protected and under conditions suitable for forming the amide linkage. The protecting group is then removed from this newly added amino acid residue and the next amino acid (suitably protected) is added, and so forth. After all the desired amino acids have been linked in the proper sequence, any remaining protecting groups (and any solid support) are removed sequentially or concurrently to afford the final peptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide.

In certain embodiments, the peptides of the invention are synthesized with amino- and carboxy-protecting groups for use as pro-drugs. Protecting groups are chemical moieties which block a reactive group on the peptide to prevent undesirable reactions. In one embodiment, a modified peptide of the invention is synthesized with one or more protecting groups that are designed to be cleaved in vivo, thereby exposing the reactive group or groups of the modified peptide to serum proteins after administration of the peptide to a subject.

The term “amino-protecting group” refers to those groups intended to protect the amino-terminal end of an amino acid or peptide or to protect the amino group of an amino acid or peptide against undesirable reactions. Commonly used amino-protecting groups are disclosed in Greene (1981) Protective Groups in Organic Synthesis (John Wiley & Sons, New York), which is hereby incorporated by reference. Additionally, protecting groups can be used which are readily cleaved in vivo, for example, by enzymatic hydrolysis, thereby exposing the amino group for reaction with serum proteins in vivo.

Amino-protecting groups comprise lower alkanoyl groups such as formyl, acetyl (“Ac”), propionyl, pivaloyl, and t-butylacetyl; other acyl groups include 2-chloroacetyl, 2-bromoacetyl, trifluoroacetyl, trichloroacetyl, phthalyl, o-nitrophenoxyacetyl, -chlorobutyryl, benzoyl, 4-chlorobenzoyl, 4-bromobenzoyl, and 4-nitrobenzoyl; sulfonyl groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-ethoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, αα-dimethyl-3,5-dimethoxybenzyloxycarbonyl, benzhydryloxycarbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, and phenylthiocarbonyl; arylalkyl groups such as benzyl, triphenylmethyl, benzyloxymethyl, 9-fluorenylmethyloxycarbonyl (Fmoc) and silyl groups such as trimethylsilyl.

The term “carboxy protecting group” refers to a carboxylic acid protecting ester or amide group employed to block or protect the carboxylic acid functionality. Carboxy protecting groups are disclosed in Greene, “Protective Groups in Organic Synthesis” pp. 152-186 (1981), which is hereby incorporated by reference. Additionally, a carboxy protecting group can be used as a pro-drug whereby the carboxy protecting group can be readily cleaved in vivo, for example by enzymatic hydrolysis, thereby exposing the carboxy group for reaction with serum proteins in vivo. Such carboxy protecting groups are well known to those skilled in the art, having been extensively used in the protection of carboxyl groups in the penicillin and cephalosporin fields as described in U.S. Pat. Nos. 3,840,556 and 3,719,667, the disclosures of which are hereby incorporated by reference.

Representative carboxy protecting groups are C₁-C₈ lower alkyl (e.g., methyl, ethyl or t-butyl); arylalkyl such as phenethyl or benzyl and substituted derivatives thereof such as alkoxybenzyl or nitrobenzyl groups; arylalkenyl such as phenylethenyl; aryl and substituted derivatives thereof such as 5-indanyl; dialkylaminoalkyl such as dimethylaminoethyl); alkanoyloxyalkyl groups such as acetoxymethyl, butyryloxymethyl, valeryloxymethyl, isobutyryloxymethyl, isovaleryloxymethyl, 1-(propionyloxy)-1-ethyl, 1-(pivaloyloxyl)-1-ethyl, 1-methyl-1-(propionyloxy)-1-ethyl, pivaloyloxymethyl, and propionyloxymethyl; cycloalkanoyloxyalkyl groups such as cyclopropylcarbonyloxymethyl, cyclobutylcarbonyloxymethyl, cyclopentylcarbonyloxymethyl, and cyclohexylcarbonyloxymethyl; aroyloxyalkyls such as benzoyloxymethyl and benzoyloxyethyl; arylalkylcarbonyloxyalkyls such as benzylcarbonyloxymethyl and 2-benzylcarbonyloxyethyl; alkoxycarbonylalkyl or cycloalkyloxycarbonylalkyl such as methoxycarbonylmethyl, cyclohexyloxycarbonylmethyl, and 1-methoxycarbonyl-1-ethyl; alkoxycarbonyloxyalkyl or cycloalkyloxycarbonyloxyalkyl such as methoxycarbonyloxymethyl, t-butyloxycarbonyloxymethyl, 1-ethoxycarbonyloxy-1-ethyl, and 1-cyclohexyloxycarbonyloxy-1-ethyl; aryloxycarbonyloxyalkyl such as 2-(phenoxycarbonyloxy)ethyl, and 2-(5-indanyloxycarbonyloxy)ethyl; alkoxyalkylcarbonyloxyalkyl such as 2-(1-methoxy-2-methylpropan-2-oyloxy)ethyl; arylalkyloxycarbonyloxyalkyl such as 2-(benzyloxycarbonyloxy)ethyl; arylalkenyloxycarbonyloxyalkyl such as 2-(3-phenylpropen-2-yloxycarbonyloxy)ethyl; alkoxycarbonylaminoalkyl such as t-butyloxycarbonylaminomethyl; alkylaminocarbonylaminoalkyl such as methylaminocarbonylaminomethyl; alkanoylaminoalkyl such as acetylaminomethyl; heterocycliccarbonyloxyalkyl such as 4-methylpiperazinylcarbonyloxymethyl; dialkylaminocarbonylalkyl such as dimethylaminocarbonylmethyl, diethylaminocarbonylmethyl; (5-(loweralkyl)-2-oxo-1,3-dioxolen4-yl)alkyl such as (5-t-butyl-2-oxo-1,3-dioxolen-4-yl)methyl; and (5-phenyl-2-oxo-1,3-dioxolen-4-yl)alkyl such as (5-phenyl-2-oxo-1,3-dioxolen-4-yl)methyl.

Preferred carboxy-protected peptides of the invention are peptides wherein the protected carboxy group is a lower alkyl, cycloalkyl or arylalkyl ester, for example, methyl ester, ethyl ester, propyl ester, isopropyl ester, butyl ester, sec-butyl ester, isobutyl ester, amyl ester, isoamyl ester, octyl ester, cyclohexyl ester, and phenylethyl ester or an alkanoyloxyalkyl, cycloalkanoyloxyalkyl, aroyloxyalkyl or an arylalkylcarbonyloxyalkyl ester. Preferred amide carboxy protecting groups are lower alkylaminocarbonyl groups. For example, aspartic acid may be protected at the α-C-terminal by an acid labile group (e.g., t-butyl) and protected at the β-C-terminal by a hydrogenation labile group (e.g., benzyl) then deprotected selectively during synthesis.

In some embodiments, the peptide or functional variant thereof consists of, or includes, an amino acid sequence located at the junctional region between two exons that are predominantly joined together in protein isoforms expressed or associated with one or more cancerous or tumor cells or disorders, e.g., as a result of an in-frame exon deletion or the use of an alternatively spliced exon. In one embodiment, the peptide or functional variant thereof consists of, or includes, an amino acid sequence identical to the alternative spliced form of Exon III, e.g., from about amino acids 301 to 360 of FGFR2-IIIc (SEQ ID NO:2); about amino acids 314 to 324 of FGFR2-IIIc (AAGVNTTDKEI, SEQ ID NO:4); about amino acids 328 to 337 of FGFR2-IIIc (YIRNVTFEDA, SEQ ID NO: 6); about amino acids 350 to 353 of FGFR2-IIIc (ISFH, SEQ ID NO: 8), or an amino acid sequence encoded by a nucleotide sequence of SEQ ID NOs: 1, 3, 5 or 7; or an amino acid or nucleotide sequence substantially identical thereto. In another embodiment, the peptide or functional variant thereof consists of, or includes, an amino acid sequence identical the junctional region between Ig-II and Ig-III of FGFR1L (SEQ ID NO: 10) or a fragment thereof, or an amino acid sequence encoded by a nucleotide sequence of SEQ ID NO: 9 or a fragment thereof; or an amino acid or nucleotide sequence substantially identical thereto. In yet other embodiments, the peptide or functional variant thereof consists of, or includes, an amino acid sequence identical to the junctional region between exon 4 and exon 7 of isoform RONΔ160 (SEQ ID NO: 12) or a fragment thereof, or an amino acid sequence encoded by a nucleotide sequence of SEQ ID NO: 11 or a fragment thereof; or an amino acid or nucleotide sequence substantially identical thereto. In yet another embodiment, the peptide or functional variant thereof consists of, or includes, an amino acid sequence identical to the junctional region of KIT between exons 10 and 12 of SEQ ID NO: 14 or a fragment thereof, or an amino acid sequence encoded by a nucleotide sequence of SEQ ID NO: 13 or a fragment thereof; or an amino acid or nucleotide sequence substantially identical thereto. In yet another embodiment, the peptide or functional variant thereof consists of, or includes, an amino acid sequence identical to the junctional region of PDGF between exons 5 and 7 of SEQ ID NO: 16 or a fragment thereof, or an amino acid sequence encoded by a nucleotide sequence of SEQ ID NO: 15 or a fragment thereof; or an amino acid or nucleotide sequence substantially identical thereto. In another embodiment, the peptide or functional variant thereof consists of, or includes, an amino acid sequence identical to the junctional region of PDGFR-alpha between exons 6 and 9 of SEQ ID NO: 18 or a fragment thereof, or an amino acid sequence encoded by a nucleotide sequence of SEQ ID NO: 17 or a fragment thereof; or an amino acid or nucleotide sequence substantially identical thereto. The peptides can be made recombinantly or synthetically, e.g., using solid phase synthesis. The isoform-binding molecule may include at least one, or alternatively, two or more peptide or variants thereof as described herein. For example, any combination of two or more peptide or peptide variants can be arranged, optionally, via a linker sequence. The peptides can be functionally linked (e.g., by chemical coupling, genetic fusion, non-covalent association or otherwise) to one or more other molecular entities, e.g., carriers (e.g., an immunoglobulin Fc domain, serum albumin, pegylation, a GST, Lex-A or an MBP polypeptide sequence) to enhance the peptide stability in vivo. Alternatively, the peptides can be modified by, e.g., addition of chemical protecting groups, to enhance the peptide stability in vivo.

### Pegylation

One widely used techniques for increasing the half-life and/or the reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). Generally, any suitable form of pegylation can be used, such as the pegylation used in the art for antibody molecules; reference is made to for example Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO 04/060965. Various reagents for pegylation of proteins are also commercially available, for example from Nektar Therapeutics, USA.

Preferably, site-directed pegylation is used, in particular via a cysteine-residue (see for example Yang et al., Protein Engineering, 16, 10, 761-770 (2003). For example, for this purpose, PEG may be attached to a cysteine residue that naturally occurs in an isoform-specific inhibitor, an inhibitor may be modified so as to suitably introduce one or more cysteine residues for attachment of PEG, or an amino acid sequence comprising one or more cysteine residues for attachment of PEG may be fused to the N- and/or C-terminus of an inhibitor of the invention, all using techniques of protein engineering known per se to the skilled person.

Preferably, for the isoform-specific inhibitor, a PEG is used with a molecular weight of more than 5000, such as more than 10,000 and less than 200,000, such as less than 100,000; for example in the range of 20,000-80,000.

With regard to pegylation, its should be noted that generally, the invention also encompasses any SDAB molecule that has been pegylated at one or more amino acid positions, preferably in such a way that said pegylation either (1) increases the half-life in vivo; (2) reduces immunogenicity; (3) provides one or more further beneficial properties known per se for pegylation; (4) does not essentially affect the affinity of the SDAB molecule (e.g. does not reduce said affinity by more than 90%, preferably not by more than 50%, and by no more than 10%, as determined by a suitable assay, such as those described in the Examples below); and/or (4) does not affect any of the other desired properties of the isoform-specific inhibitor. Suitable PEG-groups and methods for attaching them, either specifically or non-specifically, will be clear to the skilled person.

Suitable kits and reagents for such pegylation can for example be obtained from Nektar (CA, USA).

Another, usually less preferred modification comprises N-linked or O-linked glycosylation, usually as part of co-translational and/or post-translational modification, depending on the host cell used for expressing the isoform-specific inhibitor.

### Nucleic Acid Binding Molecules

In another embodiment, the isoform-specific inhibitor (e.g., the isoform-binding molecule) inhibits the expression of nucleic acid encoding the isoform, e.g., the oncogenic isoform (e.g., an oncogenic isoform as described herein). Examples of such isoform-binding molecules include nucleic acid molecules, for example, antisense molecules, ribozymes, RNAi, triple helix molecules that hybridize to a nucleic acid encoding the isoform, e.g., the oncogenic isoform, or a transcription regulatory region, and blocks or reduces mRNA expression of the isoform, e.g., the oncogenic isoform. In one embodiment, the nucleic acid binding molecule capable of inhibiting the expression of an oncogenic isoform is an antisense oligonucleotide capable of specifically hybridizing to the oncogenic isoform.

It is understood in the art that the sequence of an antisense compound need not be 100% complementary to that of its target nucleic acid to specifically hybridize to that sequence. An antisense compound specifically hybridizes to a target DNA or RNA sequence when binding of the compound to the target DNA or RNA sequence interferes with the normal function of the target DNA or RNA. This interference should cause a loss of utility, and there should be a sufficient degree of complementarity to avoid non-specific binding of the antisense compound to non-target sequences under conditions in which specific binding is desired, i.e., under physiological conditions in the case of in vivo assays or therapeutic treatment, and in case of in vitro assays, under conditions in which the assays are performed.

The sequence of an antisense oligonucleotide capable of specifically hybridizing to an oncogenic isoform can be identified through routine experimentation. In one embodiment the antisense oligonucleotide is capable of specifically hybridizing to a nucleic acid sequence provided herein, such as, e.g., a sequence encoding a polypeptide selected from the group consisting SEQ ID NOs: 2, 4, 6, 8, 10, 12, 14, 16, and 18. In another embodiment, the antisense oligonucleotide is capable of specifically hybridizing to a nucleic acid comprising the sequence of SEQ ID NO: 1, 3, 5, 7, 9, 11, 13, 15, or 17.

In another embodiment, the compound capable of inhibiting the expression of an oncogenic isoform is an RNAi construct. In one embodiment the RNAi construct is capable of specifically hybridizing to a nucleic acid sequence provided herein, such as, e.g., a sequence encoding a polypeptide selected from the group consisting SEQ ID NOs: 2, 4, 6, 8, 10, 12, 14, 16, and 18 or a substantially identical sequence thereof.

The antisense oligonucleotides and RNAi constructs can be used to specifically inhibit the expression of the oncogenic polypeptide isoforms without inhibiting the non-oncogenic polypeptide isoforms derived from the same proto-oncogene. Using this technology, the specific function of each oncogenic polypeptide isoform can be studied. Further, antisense oligonucleotides and RNAi constructs may be used for disease treatment.

Antisense oligonucleotides are relatively short nucleic acids that are complementary (or antisense) to the coding strand (sense strand) of the mRNA encoding a particular protein. Although antisense oligonucleotides are typically RNA based, they can also be DNA based. Additionally, antisense oligonucleotides are often modified to increase their stability. See, for example, Antisense Technology in Methods in Enzymology, Vols. 313-314, ed. by Phillips, Abelson and Simon, Academic Press, 1999.

The oligonucleotides can be DNA or RNA, or chimeric mixtures or derivatives or modified versions thereof, single-stranded or double-stranded. The oligonucleotide can be modified at the base moiety, sugar moiety, or phosphate backbone, for example, to improve its stability, hybridization, etc. The oligonucleotide may include other appended groups such as peptides (e.g., for targeting host cell receptors), or agents facilitating transport across the cell membrane (see, e.g., Letsinger et al., Proc. Natl. Acad. Sci. U.S.A. 86:6553-56 (1989); Lemaitre et al., Proc. Natl. Acad. Sci. U.S.A. 84:648-52 (1987); International Patent Publication No. WO88/09810) or the blood-brain barrier (see, e.g., International Patent Publication No. WO89/10134), hybridization-triggered cleavage agents (see, e.g., Krol et al., BioTechniques 6:958-76 (1988)) or intercalating agents. (see, e.g., Zon, Pharm. Res. 5:539-49 (1988)). To this end, the oligonucleotide may be conjugated to another molecule.

The antisense oligonucleotide may comprise at least one modified base moiety which may be selected from the group including, but not limited to, 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N2-carboxypropyl)uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al., Proc. Natl. Acad. Sci. U.S.A. 93:14670 (1996) and in Eglom et al. Nature 365:566 (1993). One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

Oligonucleotides of the invention may be synthesized by standard methods known in the art, e.g., by use of an automated DNA synthesizer (such as are commercially available from Biosearch Technologies, Inc. (Novato, Calif.), Applied Biosystems (Foster City, Calif.), and others). As examples, phosphorothioate oligonucleotides may be synthesized by the method of Stein et al. (Nucl. Acids Res. 16:3209 (1988)), and methylphosphonate oligonucleotides can be prepared by use of controlled pore glass polymer supports (Sarin et al., Proc. Natl. Acad. Sci. U.S.A. 85:7448-51 (1988)).

The selection of an appropriate oligonucleotide can be readily performed by one of skill in the art, based upon the present description. Given the nucleic acid encoding a particular protein, one of skill in the art can design antisense oligonucleotides that bind to that protein, and test these oligonucleotides in an in vitro or in vivo system to confirm that they bind to and mediate the degradation of the mRNA encoding the particular protein. To design an antisense oligonucleotide that specifically binds to and mediates the degradation of a particular protein, it is important that the sequence recognized by the oligonucleotide is unique or substantially unique to that particular protein. For example, sequences that are frequently repeated across proteins may not be an ideal choice for the design of an oligonucleotide that specifically recognizes and degrades a particular message. One of skill in the art can design an oligonucleotide, and compare the sequence of that oligonucleotide to nucleic acid sequences that are deposited in publicly available databases to confirm that the sequence is specific, or substantially specific, for a particular protein.

A number of methods have been developed for delivering antisense DNA or RNA to cells, e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically to a subject. See, for example, Antisense Technology in Methods in Enzymology, Vols. 313-314, ed. by Phillips, Abelson and Simon, Academic Press, 1999.

RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by any particular theory, RNAi appears to involve mRNA degradation; however, the biochemical mechanisms remain an active area of research.

As used herein, the term “dsRNA” refers to siRNA molecules, or other RNA molecules including a double stranded feature and able to be processed to siRNA in cells, such as hairpin RNA moieties.

As used herein, the term “RNAi construct” is a generic term used throughout the specification to include small interfering RNAs (siRNAs), hairpin RNAs, and other RNA species, which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts, which can produce siRNAs in vivo.

“RNAi expression vector” (also referred to herein as a “dsRNA-encoding plasmid”) refers to a replicable nucleic acid constructs used to express (transcribe) RNA, which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids” which refer to circular double stranded DNA loops, which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

The sequence identity between the RNAi construct and a target sequence may be optimized by sequence comparison and alignment algorithms known in the art (see, Gribskov and Devereux, Sequence Analysis Primer, Stockton Press, 1991, and references cited therein) and by calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., using hybridization conditions such as 400 mM NaCl, 40 mM PIPES pH 6.4, 1 mM EDTA, 50° C. or 70° C. for 12-16 hours; followed by washing).

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, e.g., Heidenreich et al., Nucleic Acids Res. 25:776-80 (1997); Wilson et al., J. Mol. Recog. 7:89-98 (1994); Chen et al., Nucleic Acids Res. 23:2661-68 (1995); Hirschbein et al., Antisense Nucleic Acid Drug Dev. 7:55-61 (1997)). Merely to illustrate, the backbone of an RNAi construct can be modified with phosphorothioates, phosphoramidate, phosphodithioates, chimeric methylphosphonate-phosphodie-sters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount, which allows delivery of at least one copy per cell. Higher doses (e.g., at least 5, 10, 100, 500 or 1000 copies per cell) of double-stranded material may yield more effective inhibition, while lower doses may also be useful for specific applications Inhibition is sequence-specific in that nucleotide sequences corresponding to the duplex region of the RNA are targeted for genetic inhibition.

In certain embodiments, the subject RNAi constructs is “small interfering RNAs” or “siRNAs.” These nucleic acids may be around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotide-long siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen et al., Proc. Natl. Acad. Sci. U.S.A., 98:9742-47 (2001); Elbashir et al., EMBO J., 20:6877-88 (2001)). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain preferred embodiments, at least one strand of the siRNA molecules has a 3′ overhang from about 1 to about 6 nucleotides in length, preferably from 2 to 4 nucleotides in length. More preferably, the 3′ overhangs are 1-3 nucleotides in length. In certain embodiments, one strand has a 3′ overhang and the other strand is blunt-ended or also has an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, in some embodiments, the uses of local delivery systems and/or agents, which reduce the effects of interferon or PKR, are preferred.

In certain embodiments, the RNAi construct is in the form of a hairpin structure (named as hairpin RNA). The hairpin RNAs can be synthesized exogenously or can be formed by transcribing from RNA polymerase III or RNA polymerase II promoters in vivo. Examples of making and using such hairpin RNAs for gene silencing in mammalian cells are described in, for example, Paddison et al., Genes Dev, 16:948-58 (2002); McCaffrey et al., Nature, 418:38-39 (2002); McManus et al., RNA 8:842-50 (2002); Yu et al., Proc Natl Acad Sci U.S.A., 99:6047-52 (2002)). Preferably, such hairpin RNAs are engineered in cells or in an animal to ensure continuous and stable suppression of a desired gene. It is known in the art that siRNAs can be produced by processing a hairpin RNA in the cell. In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

International Patent Publication No. WO 01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

Exemplary RNAi constructs that specifically recognize a particular gene, or a particular family of genes can be selected using methodology outlined in detail herein with respect to the selection of antisense oligonucleotide. Similarly, methods of delivery RNAi constructs include the methods for delivery antisense oligonucleotides outlined in detail herein.

The invention also provides methods of inhibiting the expression of an oncogenic isoform provided herein in a cell comprising contacting the cell with a compound capable of inhibiting the expression of the oncogenic isoform Inhibition of expression of an oncogenic isoform may be useful for the prevention and/or treatment of cancer Inhibiting expression of an FGFR2-IIIc oncogenic isoform may be used to prevent and/or treat hormone-refractory prostate cancer, breast cancer, bladder cancer, thyroid cancer, or other form of cancer Inhibiting expression of FGFR1L may be used to prevent and/or treat pancreatic adenocarcinoma, prostate cancer, or other form of cancer. Inhibiting expression of a RON receptor tyrosine kinase Δ160 isoform may be used to prevent and/or treat metastatic colorectal cancer, breast cancer, ovarian cancer, lung cancer, bladder cancer, or other form of cancer Inhibiting expression of a KIT receptor tyrosine kinase oncogenic isoform may be used to prevent and/or treat gastrointestinal stromal tumors (GISTs) or other form of cancer Inhibiting expression of a PDGFR-alpha isoform may be used to prevent and/or treat brain cancer, glioblastoma, prostate cancer, bone metastasis, GIST, or other form of cancer.

In one embodiment the method is carried out in vitro. In another embodiment the method will be carried out in vivo. These methods could be used in research, diagnosis and treatment of a cancer associated with expression of the oncogenic isoform. In research, these methods could be used, for example, to elucidate the mechanism of action of an oncogenic isoform of the invention.

### Pharmaceutical Compositions and Kits

In another aspect, the present invention provides compositions, e.g., pharmaceutically acceptable compositions, which include an isoform-specific inhibitor described herein, formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g. by injection or infusion).

The compositions of this invention may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical preferred compositions are in the form of injectable or infusible solutions. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The isoform-specific inhibitor of the present invention can be administered by a variety of methods known in the art, although for many therapeutic applications, the preferred route/mode of administration is intravenous injection or infusion. For example, the antibody molecules can be administered by intravenous infusion at a rate of less than 10 mg/min; preferably less than or equal to 5 mg/min to reach a dose of about 1 to 100 mg/m², preferably about 5 to 50 mg/m², about 7 to 25 mg/m² and more preferably, about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an isoform-specific inhibitor of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of an antibody or antibody portion of the invention is 0.1-20 mg/kg, more preferably 1-10 mg/kg. The isoform-specific inhibitor can be administered by intravenous infusion at a rate of less than 10 mg/min, preferably less than or equal to 5 mg/min to reach a dose of about 1 to 100 mg/m², preferably about 5 to 50 mg/m², about 7 to 25 mg/m², and more preferably, about 10 mg/m². It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the modified antibody or antibody fragment may vary according to factors such as the disease state, age, sex, and weight of the individual, and the ability of the antibody or antibody portion to elicit a desired response in the individual. A therapeutically effective amount is also one in which any toxic or detrimental effects of the modified antibody or antibody fragment is outweighed by the therapeutically beneficial effects. A “therapeutically effective dosage” preferably inhibits a measurable parameter, e.g., tumor growth rate by at least about 20%, more preferably by at least about 40%, even more preferably by at least about 60%, and still more preferably by at least about 80% relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the invention is a kit comprising an isoform-specific inhibitor. The kit can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject. Instructions for use can include instructions for diagnostic applications of the isoform-binding molecule, in vitro, e.g., in a sample, e.g., a biopsy or cells from a patient having a cancer or prostatic disorder, or in vivo. The instructions can include instructions for therapeutic application including suggested dosages and/or modes of administration, e.g., in a patient with a cancer or prostatic disorder. Other instructions can include instructions on coupling of the antibody to a chelator, a label or a therapeutic agent, or for purification of a conjugated antibody, e.g., from unreacted conjugation components. As discussed above, the kit can include a label, e.g., any of the labels described herein. As discussed above, the kit can include a therapeutic agent, e.g., a therapeutic agent described herein. The kit can include a reagent useful for chelating or otherwise coupling a label or therapeutic agent to the antibody, e.g., a reagent discussed herein. For example, a macrocyclic chelating agent, preferably 1,4,7,10-tetraazacyclododecane-N,N′,N″,N′″,4-tetraacetic acid (DOTA), can be included. The DOTA can be supplied as a separate component or the DOTA (or other chelator or conjugating agent) can be supplied already coupled to the antibody. Additional coupling agents, e.g., an agent such as N-hydroxysuccinimide (NHS), can be supplied for coupling the chelator, e.g., DOTA, to the antibody. In some applications the antibody will be reacted with other components; e.g., a chelator or a label or therapeutic agent, e.g., a radioisotope, e.g., yttrium or lutetium. In such cases the kit can include one or more of a reaction vessel to carry out the reaction or a separation device, e.g., a chromatographic column, for use in separating the finished product from starting materials or reaction intermediates.

The kit can further contain at least one additional reagent, such as a diagnostic or therapeutic agent, e.g., a diagnostic or therapeutic agent as described herein, and/or one or more additional isoform-specific inhibitor, formulated as appropriate, in one or more separate pharmaceutical preparations.

The kit can further contain a radioprotectant. The radiolytic nature of isotopes, e.g., ⁹⁰Yttrium (⁹⁰Y) is known. In order to overcome this radiolysis, radioprotectants may be included, e.g., in the reaction buffer, as long as such radioprotectants are benign, meaning that they do not inhibit or otherwise adversely affect the labeling reaction, e.g., of an isotope, such as of ⁹⁰Y, to the antibody.

The formulation buffer of the present invention may include a radioprotectant such as human serum albumin (HSA) or ascorbate, which minimize radiolysis due to yttrium or other strong radionuclides. Other radioprotectants are known in the art and can also be used in the formulation buffer of the present invention, i.e., free radical scavengers (phenol, sulfites, glutathione, cysteine, gentisic acid, nicotinic acid, ascorbyl palmitate, HOP(:O)H₂I glycerol, sodium formaldehyde sulfoxylate, Na₂S₂0, Na₂S₂0₃, and S0₂, etc.).

A preferred kit is one useful for radiolabeling a chelator-conjugated protein or peptide with a therapeutic radioisotope for administration to a patient. The kit includes (i) a vial containing chelator-conjugated antibody, (ii) a vial containing formulation buffer for stabilizing and administering the radiolabeled antibody to a patient, and (iii) instructions for performing the radiolabeling procedure. The kit provides for exposing a chelator-conjugated antibody to the radioisotope or a salt thereof for a sufficient amount of time under amiable conditions, e.g., as recommended in the instructions. A radiolabeled antibody having sufficient purity, specific activity and binding specificity is produced. The radiolabeled antibody may be diluted to an appropriate concentration, e.g., in formulation buffer, and administered directly to the patient with or without further purification. The chelator-conjugated antibody may be supplied in lyophilized form.

### Uses of the Invention

The isoform-specific inhibitors of the invention have in vitro and in vivo diagnostic, as well as therapeutic and prophylactic utilities. For example, these binding molecules can be administered to cells in culture, e.g. in vitro or ex vivo, or in a subject, e.g., in vivo, to treat, prevent, and/or diagnose a variety of disorders, such as cancers (prostatic and non-prostatic cancers). As used herein, the term “subject” is intended to include human and non-human animals. Preferred human animals include a human patient having a disorder characterized by abnormal functioning of an isoform-expressing cell, e.g., a cancer cell or a prostatic cell. The term “non-human animals” of the invention includes all vertebrates, e.g., mammals and non-mammals, such as non-human primates, sheep, dog, cow, chickens, amphibians, reptiles, etc.

In one embodiment, the subject is a human subject. Alternatively, the subject can be a mammal expressing an isoform-like antigen with which an isoform-specific inhibitor of the invention cross-reacts. An isoform-specific inhibitor of the invention can be administered to a human subject for therapeutic purposes (discussed further below). Moreover, an isoform-specific inhibitor can be administered to a non-human mammal expressing the isoform-like antigen with which the modified antibody cross-reacts (e.g., a primate, pig or mouse) for veterinary purposes or as an animal model of human disease. Regarding the latter, such animal models may be useful for evaluating the therapeutic efficacy of antibodies of the invention (e.g., testing of dosages and time courses of administration).

### Therapeutic Uses

In one embodiment, the invention provides a method of treating, e.g., ablating or killing, a hyperproliferative cell, e.g., a prostatic cell (e.g., a cancerous prostatic), or a malignant, non-prostatic cell, e.g., cell found in a non-prostatic solid tumor, a soft tissue tumor, or a metastatic lesion (e.g., a cell found in renal, urothelial (e.g., bladder), testicular, colon, rectal, lung (e.g., non-small cell lung carcinoma), breast, liver, neural (e.g., neuroendocrine), glial (e.g., glioblastoma), pancreatic (e.g., pancreatic duct) cancer and/or metastasis, melanoma (e.g., malignant melanoma), or soft tissue sarcoma). Methods of the invention include the steps of contacting the hyperproliferative cell, with an isoform-specific inhibitor described herein, in an amount sufficient to treat, e.g., reduce the activity, ablate or kill, the hyperproliferative cell.

The subject method can be used on cells in culture, e.g. in vitro or ex vivo. For example, cancerous or metastatic cells (e.g., prostatic, renal, an urothelial, colon, rectal, lung, breast or liver, cancerous or metastatic cells) can be cultured in vitro in culture medium and the contacting step can be effected by adding the isoform-specific inhibitor, to the culture medium. The method can be performed on cells (e.g., cancerous or metastatic cells) present in a subject, as part of an in vivo (e.g., therapeutic or prophylactic) protocol. For in vivo embodiments, the contacting step is effected in a subject and includes administering the isoform-specific inhibitor to the subject under conditions effective to permit inhibiting and/or reducing one or more activities of the isoform, or binding of the isoform-binding molecule to the cell, and thereby treating, e.g., the killing or ablating of the cell.

As used herein, the term “cancer” is meant to include all types of cancerous growths or oncogenic processes, metastatic tissues or malignantly transformed cells, tissues, or organs, irrespective of histopathologic type or stage of invasiveness. Examples of cancerous disorders include, but are not limited to, solid tumors, soft tissue tumors, and metastatic lesions. Examples of solid tumors include malignancies, e.g., sarcomas, adenocarcinomas, and carcinomas, of the various organ systems, such as those affecting prostate, lung, breast, lymphoid, gastrointestinal (e.g., colon), and genitourinary tract (e.g., renal, urothelial cells), pharynx. Adenocarcinomas include malignancies such as most colon cancers, rectal cancer, renal-cell carcinoma, liver cancer, non-small cell carcinoma of the lung, cancer of the small intestine and cancer of the esophagus. Metastatic lesions of the aforementioned cancers can also be treated or prevented using the methods and compositions of the invention.

The subject method can be useful in treating malignancies of the various organ systems, such as those affecting lung, breast, lymphoid, gastrointestinal (e.g., colon), bladder, genitourinary tract (e.g., prostate), pharynx, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

Methods of administering the isoform-specific inhibitors of the invention are described above. Suitable dosages of the molecules used will depend on the age and weight of the subject and the particular drug used. The modified antibody molecules can be used as competitive agents for ligand binding to inhibit, reduce an undesirable interaction.

The isoform-specific inhibitors of the invention can be used by themselves or conjugated to a second agent, e.g., a cytotoxic drug, radioisotope, or a protein, e.g., a protein toxin or a viral protein. This method includes: administering the isoform-specific inhibitors, alone or conjugated to a cytotoxic drug, to a subject requiring such treatment.

The isoform-specific inhibitors of the invention may be used to deliver a variety of therapeutic agents, e.g., a cytotoxic moiety, e.g., a therapeutic drug, a radioisotope, molecules of plant, fungal, or bacterial origin, or biological proteins (e.g., protein toxins) or particles (e.g., a recombinant viral particles, e.g.; via a viral coat protein), or mixtures thereof. The therapeutic agent can be an intracellularly active drug or other agent, such as short-range radiation emitters, including, for example, short-range, high-energy a-emitters, as described herein. In some embodiments, the isoform-specific inhibitors of the invention can be coupled to a molecule of plant or bacterial origin (or derivative thereof), e.g., a maytansinoid. Maytansine is a cytotoxic agent that effects cell killing by preventing the formation of microtubules and depolymerization of extant microtubules. It is 100- to 1000-fold more cytotoxic than anticancer agents such as doxorubicin, methotrexate, and vinca alkyloid, which are currently in clinical use. Alternatively, the isoform-binding molecule can be coupled to a taxane, a calicheamicin, a proteosome inhibitor, or a topoisomerase inhibitor. [(1R)-3-methyl-1-[[(2S)-1-oxo-3-phenyl-2-[(3-mercaptoacetyl) amino]propyl]amino]butyl] Boronic acid is a suitable proteosome inhibitor. N,N′-bis[2-(9-methylphenazine-1-carboxamido)ethyl]-1,2-ethanediamine is a suitable topoisomerase inhibitor.

Enzymatically active toxins and fragments thereof are exemplified by diphtheria toxin A fragment, nonbinding active fragments of diphtheria toxin, exotoxin A (from Pseudomonas aeruginosa), ricin A chain, abrin A chain, modeccin A chain, α-sacrin, certain Aleurites fordii proteins, certain Dianthin proteins, Phytolacca americana proteins (PAP, PAPII and PAP-S), Morodica charantia inhibitor, curcin, crotin, Saponaria officinalis inhibitor, gelonin, mitogillin, restrictocin, phenomycin, and enomycin. In one embodiment, the isoform-binding molecule is conjugated to maytansinoids, e.g., maytansinol (see U.S. Pat. No. 5,208,020), CC-1065 (see U.S. Pat. Nos. 5,475,092, 5,585,499, 5,846,545). Procedures for preparing enzymatically active polypeptides of the immunotoxins are described in WO84/03508 and WO85/03508, which are hereby incorporated by reference. Examples of cytotoxic moieties that can be conjugated to the antibodies include adriamycin, chlorambucil, daunomycin, methotrexate, neocarzinostatin, and platinum.

To kill or ablate cancerous prostate epithelial cells, a first isoform-binding molecule can be conjugated with a prodrug which is activated only when in close proximity with a prodrug activator. The prodrug activator is conjugated with a second isoform-binding molecule according to the present invention, preferably one that binds to a non-competing site on the prostate specific membrane antigen molecule. Whether two modified antibodies bind to competing or non-competing binding sites can be determined by conventional competitive binding assays. Drug-prodrug pairs suitable for use in the practice of the present invention are described in Blakely et al., “ZD2767, an Improved System for Antibody-directed Enzyme Prodrug Therapy That Results in Tumor Regressions in Colorectal Tumor Xenografts,” (1996) Cancer Research, 56:3287-3292, which is hereby incorporated by reference.

Alternatively, the isoform-binding molecules of the invention can be coupled to high energy radiation emitters, for example, a radioisotope, such as ¹³¹I, a γ-emitter, which, when localized at the tumor site, results in a killing of several cell diameters. See, e.g., S. E. Order, “Analysis, Results, and Future Prospective of the Therapeutic Use of Radiolabeled Antibody in Cancer Therapy”, Monoclonal Antibodies for Cancer Detection and Therapy, R. W. Baldwin et al. (eds.), pp 303-316 (Academic Press 1985), which is hereby incorporated by reference. Other suitable radioisotopes include a-emitters, such as ²¹²Bi, ²¹³Bi, and ²¹¹At, and β-emitters, such as ¹⁸⁶Re and ⁹⁰Y. Radiotherapy is expected to be particularly effective, because prostate epithelial cells and vascular endothelial cells within cancers are relatively radiosensitive. Moreover, Lu¹¹⁷ may also be used as both an imaging and cytotoxic agent.

Radioimmunotherapy (RIT) using antibodies labeled with ¹³¹I, ⁹⁰Y, and ¹¹⁷Lu is under intense clinical investigation. There are significant differences in the physical characteristics of these three nuclides and as a result, the choice of radionuclide can be important in order to deliver maximum radiation dose to the tumor. The higher beta energy particles of ⁹⁰Y may be good for bulky tumors, but it may not be necessary for small tumors and especially bone metastases, (e.g. those common to prostate cancer). The relatively low energy beta particles of ¹³¹I are ideal, but in vivo dehalogenation of radioiodinated molecules is a major disadvantage for internalizing antibody. In contrast, ¹⁷⁷Lu has low energy beta particle with only 0.2-0.3 mm range and delivers much lower radiation dose to bone marrow compared to ⁹⁰Y. In addition, due to longer physical half-life (compared to ⁹⁰Y), the tumor residence times are higher. As a result, higher activities (more mCi amounts) of ¹⁷⁷Lu labeled agents can be administered with comparatively less radiation dose to marrow. There have been several clinical studies investigating the use of ¹⁷⁷Lu labeled antibodies in the treatment of various cancers. (Mulligan T et al., (1995) Clin Cancer Res. 1:1447-1454; Meredith R F, et al. (1996) J Nucl Med 37:1491-1496; Alvarez R D, et al. (1997) Gynecologic Oncology 65: 94-101).

The isoform-specific inhibitors of the invention can also be conjugated or fused to viral surface proteins present on viral particles. For example, an isoform-binding molecule of the invention could be fused (e.g., to form a fusion protein) to a viral surface protein. Alternatively, a whole isoform-specific inhibitor could be chemically conjugated (e.g., via a chemical linker) to a viral surface protein. Preferably, the virus is one that fuses with endocytic membranes, e.g., an influenza virus, such that the virus is internalized along with the isoform-specific inhibitor and thereby infects isoform-expressing cells. The virus can be genetically engineered as a cellular toxin. For example, the virus could express or induce the expression of genes that are toxic to cells, e.g., cell death promoting genes. Preferably, such viruses would be incapable of viral replication.

The isoform-specific inhibitors of the invention can be used directly in vivo to eliminate antigen-expressing cells via natural complement or antibody-dependent cellular cytotoxicity (ADCC). Isoform-specific inhibitors of the invention, which have complement binding sites, such as portions from IgG1, -2, or -3 or IgM which bind complement can also be used in the presence of complement. In one embodiment, ex vivo treatment of a population of cells comprising target cells with a binding agent of the invention and appropriate effector cells can be supplemented by the addition of complement or serum containing complement. Phagocytosis of target cells coated with modified antibodies or fragments thereof of the invention can be improved by binding of complement proteins. In another embodiment, target cells coated with the isoform-specific inhibitors of the invention can also be lysed by complement.

Also encompassed by the present invention is a method of killing or ablating cells which involves using the isoform-specific inhibitors of the invention for preventing an isoform-related disorder. For example, these materials can be used to prevent or delay development or progression of prostate or other cancers.

Use of the therapeutic methods of the present invention to treat prostate and other cancers has a number of benefits. Since isoform-specific inhibitors according to the present invention only target cancerous cells, other tissue is spared. As a result, treatment with such isoform-specific inhibitors is safer, particularly for elderly patients. Treatment according to the present invention is expected to be particularly effective, because it directs high levels of isoform-specific inhibitors to the bone marrow and lymph nodes where prostate cancer metastases and metastases of many other cancers predominate. Moreover, the methods of the present invention are particularly well-suited for treating prostate cancer, because tumor sites for prostate cancer tend to be small in size and, therefore, easily destroyed by cytotoxic agents. Treatment in accordance with the present invention can be effectively monitored with clinical parameters, such as, in the case of prostate cancer, one or more markers chosen from: serum PSA, PSMA, PSCA, AR, chromogranin, synaptophysin, MIB-1, and/or AMACR), and/or pathological features of a patient's cancer, including stage, Gleason score, extracapsular, seminal, vesicle or perineural invasion, positive margins, involved lymph nodes, disease related pain, etc. Alternatively, these parameters can be used to indicate when such treatment should be employed.

Also provided herein are DNA vaccines comprising a nucleotide sequence encoding an epitope of an oncogenic polypeptide isoform, which may be used for the prevention or treatment of cancer. The epitope may be a short peptide of 10-15 amino acid residues from a linear or non-linear sequence of an oncogenic polypeptide isoform. The epitope preferably spans a junction site between two exons, which junction is unique to the particular polypeptide isoform that is associated with cancer and not present in the protein isoform that is found in normal subjects or in normal tissues of diseases subjects. In certain embodiments, DNA vaccines will encode two or more epitopes from a single protein isoform or from multiple protein isoforms and may be used in such combination, e.g., for certain disease indications. DNA vaccines may also encode an epitope specific sequence, e.g., encoding 10-15 amino acids, fused in frame to a carrier protein such as serum albumin, SEAP or other secreted peptide or protein. DNA vaccines may be used for preventing or treated diseases as further described herein. Exemplary DNA vaccines comprise nucleotide sequences encoding peptides of sequences described herein, or identified as described herein.

To test the efficacy of a DNA vaccine, the vaccine may be given to an experimental animal model. Animal models are well known in the art for numerous diseases, for example, for human tumors. In an illustrative embodiment, a vaccinated animal will be challenged with inoculated human tumors either before or after vaccination with a DNA vaccine. A protective or positive effect of the vaccine should be reflected by reduced tumor burden in the experimental animals. Without wanting to be limited to a particular mechanism of action, a tumor-specific vaccine may stimulate either one or both body's immune arms, i.e. cellular immunity and humoral immunity.

### Combination Therapy

The isoform-specific inhibitors of the invention may be used in combination with other therapies. For example, the combination therapy can include a composition of the present invention co-formulated with, and/or co-administered with, one or more additional therapeutic agents, e.g., one or more anti-cancer agents, cytotoxic or cytostatic agents, hormone treatment, vaccines, and/or other immunotherapies. In other embodiments, the isoform-specific inhibitors are administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

Isoform-specific inhibitors of the invention can be administered in combination with one or more of the existing modalities for treating prostate cancers, including, but not limited to: surgery (e.g., radical prostatectomy); radiation therapy (e.g., external-beam therapy which involves three dimensional, conformal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy); hormonal therapy, which can be administered before or following radical prostatectomy or radiation (e.g., treatments which reduce serum testosterone concentrations, or inhibit testosterone activity, e.g., administering a leuteinizing hormone-releasing hormone (LHRH) analog or agonist (e.g., Lupron, Zoladex, leuprolide, buserelin, or goserelin) or antagonists (e.g., Abarelix). Non-steroidal anti-androgens, e.g., flutamide, bicalutimade, or nilutamide, can also be used in hormonal therapy, as well as steroidal anti-androgens (e.g., cyproterone acetate or megastrol acetate), estrogens (e.g., diethylstilbestrol), PROSCAR®, secondary or tertiary hormonal manipulations (e.g., involving corticosteroids (e.g., hydrocortisone, prednisone, or dexamethasone), ketoconazole, and/or aminogluthethimide), inhibitors of 5a-reductase (e.g., finisteride), herbal preparations (e.g., PC-SPES), hypophysectomy, and adrenalectomy. Furthermore, hormonal therapy can be performed intermittently or using combinations of any of the above treatments, e.g., combined use of leuprolide and flutamide.

In other embodiments, the isoform-specific inhibitors of the invention are administered in combination with an immunomodulatory agent, e.g., IL-1, 2, 4, 6, or 12, or interferon alpha or gamma. For example, the combination of antibodies having a human constant regions and IL-2 potentially is expected to enhance the efficacy of the monoclonal antibody. IL-2 will function to augment the reticuloendothelial system to recognize antigen-antibody complexes by its effects on NK cells and macrophages. Thus, by stimulating NK cells to release IFN, GM-CSF, and TNF, these cytokines will increase the cell surface density of Fc receptors, as well as the phagocytic capacities of these cells. Therefore, the effector arm of both the humoral and cellular arms will be artificially enhanced. The net effect will be to improve the efficiency of monoclonal antibody therapy, so that a maximal response may be obtained. A small number of clinical trials have combined IL-2 with a monoclonal antibody (Albertini et al. (1997) Clin Cancer Res 3: 1277-1288; Frost et al. (1997) Cancer 80:317-333; Kossman et al. (1999) Clin Cancer Res 5:2748-2755). IL-2 can be administered by either bolus or continuous infusion. Accordingly, the antibodies of the invention can be administered in combination with IL-2 to maximize their therapeutic potential.

### Diagnostic Uses

In one aspect, the present invention provides a diagnostic method for detecting the presence of an isoform, e.g., an isoform protein in vitro (e.g., in a biological sample, such as a tissue biopsy, e.g., from a cancerous tissue) or in vivo (e.g., in vivo imaging in a subject). The method includes: (i) contacting the sample with an isoform-binding molecule described herein (e.g., an anti-FGFR2-IIIc antibody molecule described herein), or administering to the subject, the isoform-binding molecule; (optionally) (ii) contacting a reference sample, e.g., a control sample (e.g., a control biological sample, such as plasma, tissue, biopsy) or a control subject)); and (iii) detecting formation of a complex between the isoform-binding molecule, and the sample or subject, or the control sample or subject, wherein a change, e.g., a statistically significant change, in the formation of the complex in the sample or subject relative to the control sample or subject is indicative of the presence of isoform in the sample. The isoform-binding molecule can be directly or indirectly labeled with a detectable substance to facilitate detection of the bound or unbound antibody. Suitable detectable substances include various enzymes, prosthetic groups, fluorescent materials, luminescent materials and radioactive materials, as described above and described in more detail below.

The term “sample,” as it refers to samples used for detecting polypeptides includes, but is not limited to, cells, cell lysates, proteins or membrane extracts of cells, body fluids, or tissue samples.

Complex formation between the isoform-binding molecule and the isoform can be detected by measuring or visualizing either the binding molecule bound to the isoform antigen or unbound binding molecule. Conventional detection assays can be used, e.g., an enzyme-linked immunosorbent assays (ELISA), a radioimmunoassay (RIA) or tissue immunohistochemistry. Alternative to labeling the isoform-binding molecule, the presence of the isoform can be assayed in a sample by a competition immunoassay utilizing standards labeled with a detectable substance and an unlabeled isoform-binding molecule. In this assay, the biological sample, the labeled standards and the binding molecule are combined and the amount of labeled standard bound to the unlabeled binding molecule is determined. The amount of isoform in the sample is inversely proportional to the amount of labeled standard bound to the binding molecule.

In still another embodiment, the invention provides a method for detecting the presence of isoform-expressing cancerous tissues in vivo. The method includes (i) administering to a subject (e.g., a patient having a cancer) an isoform-binding molecule conjugated to a detectable marker; (ii) exposing the subject to a means for detecting said detectable marker to the isoform-expressing tissues or cells. In one embodiment, the binding molecule capable of specifically binding the polypeptide oncogenic isoform is an antibody molecule described above. In another embodiment, the binding molecule is an anti-FGFR2-IIIc antibody molecule described herein. In one embodiment, the antibody specifically binds a polypeptide comprising an amino acid sequence set forth in SEQ ID NO: 2, 4, 6, 8, 10, 12, 14, 16, or 18 or a substantially identical sequence thereof.

Determining whether a subject is expressing an oncogenic isoform may be useful to diagnose cancer. Determining whether a subject is expressing an FGFR2-IIIc oncogenic isoform may be used to diagnose hormone-refractory prostate cancer, breast cancer, bladder cancer, thyroid cancer, or other form of cancer. Determining whether a subject is expressing FGFR1L may be used to diagnose pancreatic adenocarcinoma, prostate cancer, or other form of cancer. Determining whether a subject is expressing a RON receptor tyrosine kinase Δ160 isoform may be used to diagnose metastatic colorectal cancer, breast cancer, ovarian cancer, lung cancer, bladder cancer, or other form of cancer. Determining whether a subject is expressing a KIT receptor tyrosine kinase oncogenic isoform may be used to diagnose gastrointestinal stromal tumors (GISTs) or other form of cancer. Determining whether a subject is expressing a PDGFR-alpha isoform cancer may be used to diagnose brain cancer, glioblastoma, prostate cancer, bone metastasis, GIST, or other form of cancer.

When no compound is determined to have bound at a significant level an oncogenic polypeptide isoform, a negative diagnosis is made. When the compound is determined to have bound at a significant level an oncogenic polypeptide isoform, a positive diagnosis is made.

Examples of labels useful for diagnostic imaging in accordance with the present invention are radiolabels such as ¹³¹I, ¹¹¹In, ¹²³I, ^{99m}Tc, ³²P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes, such as a transrectal probe, can also be employed. These isotopes and transrectal detector probes, when used in combination, are especially useful in detecting prostatic fossa recurrences and pelvic nodal disease. The modified antibody can be labeled with such reagents using techniques known in the art. For example, see Wensel and Meares (1983) Radioimmunoimaging and Radioimmunotherapy, Elsevier, N.Y., which is hereby incorporated by reference, for techniques relating to the radiolabeling of antibodies. See also, D. Colcher et al. (1986) Meth. Enzymol. 121: 802-816, which is hereby incorporated by reference.

In the case of a radiolabeled modified antibody, the modified antibody is administered to the patient, is localized to the tumor bearing the antigen with which the modified antibody reacts, and is detected or “imaged” in vivo using known techniques such as radionuclear scanning using e.g., a gamma camera or emission tomography. See e.g., A. R. Bradwell et al., “Developments in Antibody Imaging”, Monoclonal Antibodies for Cancer Detection and Therapy, R. W. Baldwin et al., (eds.), pp 65785 (Academic Press 1985), which is hereby incorporated by reference. Alternatively, a positron emission transaxial tomography scanner, such as designated Pet VI located at Brookhaven National Laboratory, can be used where the radiolabel emits positrons (e.g., ¹¹C, ¹⁸F, ¹⁵O, and ¹³N).

Fluorophore and chromophore labeled modified antibodies can be prepared from standard moieties known in the art. Since antibodies and other proteins absorb light having wavelengths up to about 310 nm, the fluorescent moieties should be selected to have substantial absorption at wavelengths above 310 nm and preferably above 400 nm A variety of suitable fluorescent compounds and chromophores are described by Stryer (1968) Science, 162:526 and Brand, L. et al. (1972) Annual Review of Biochemistry, 41:843-868, which are hereby incorporated by reference. The isoform-binding molecule can be labeled with fluorescent chromophore groups by conventional procedures such as those disclosed in U.S. Pat. Nos. 3,940,475, 4,289,747, and 4,376,110, which are hereby incorporated by reference.

One group of fluorescers having a number of the desirable properties described above is the xanthene dyes, which include the fluoresceins derived from 3,6-dihydroxy-9-henylxanthhydrol and resamines and rhodamines derived from 3,6-diamino-9-phenylxanthydrol and lissanime rhodamine B. The rhodamine and fluorescein derivatives of 9-o- carboxyphenylxanthhydrol have a 9-o-carboxyphenyl group. Fluorescein compounds having reactive coupling groups such as amino and isothiocyanate groups such as fluorescein isothiocyanate and fluorescamine are readily available. Another group of fluorescent compounds are the naphthylamines, having an amino group in the α or β position.

In other embodiments, the invention provide methods for determining the dose, e.g., radiation dose, that different tissues are exposed to when a subject, e.g., a human subject, is administered an isoform-binding molecule that is conjugated to a radioactive isotope. The method includes: (i) administering an isoform-binding molecule as described herein, e.g., an isoform-binding molecule, that is labeled with a radioactive isotope to a subject; (ii) measuring the amount of radioactive isotope located in different tissues, e.g., prostate, liver, kidney, or blood, at various time points until some or all of the radioactive isotope has been eliminated from the body of the subject; and (iii) calculating the total dose of radiation received by each tissue analyzed. The measurements can be taken at scheduled time points, e.g., day 1, 2, 3, 5, 7, and 12, following administration (at day 0) of the radioactively labeled isoform-binding molecule to the subject. The concentration of radioisotope present in a given tissue, integrated over time, and multiplied by the specific activity of the radioisotope can be used to calculate the dose that a given tissue receives. Pharmacological information generated using isoform-binding molecules labeled with one radioactive isotope, e.g., a gamma-emitter, e.g., ¹¹¹In, can be used to calculate the expected dose that the same tissue would receive from a different radioactive isotope which cannot be easily measured, e.g., a beta-emitter, e.g., ⁹⁰Y.

### Pharmacogenomics

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, gene expression, and protein biomarker expression analysis to drugs in clinical development and on the market. See, for example, Eichelbaum, M. et al. (1996) Clin. Exp. Pharmaco. Physiol. 23:983-985 and Linder, M. W. et al. (1997) Clin. Chem. 43:254-266. Differences in metabolism of therapeutics can lead to severe toxicity or therapeutic failure by altering the relation between dose and blood concentration of the pharmacologically active drug. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. More specifically, the term refers the study of how a patient's genes determine his or her response to a drug (e.g., a patient's “drug response phenotype,” or “drug response genotype.”) Thus, another aspect of the invention provides methods for tailoring an individual's prophylactic or therapeutic treatment according to that individual's drug response genotype.

Information generated from pharmacogenomic research can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when administering a therapeutic composition, e.g., a composition consisting of one or more isoform-specific inhibitors, or derivatized form(s) thereof, to a patient, as a means of treating a disorder, e.g., a cancer as described herein.

In one embodiment, a physician or clinician may consider applying knowledge obtained in relevant pharmacogenomics studies when determining whether to administer a pharmaceutical composition, e.g., a composition consisting of one or more isoform-specific inhibitors, derivatized form(s) thereof, and optionally a second agent, to a subject. In another embodiment, a physician or clinician may consider applying such knowledge when determining the dosage, e.g., amount per treatment or frequency-of treatments, of a pharmaceutical composition, e.g., a pharmaceutical composition as described herein, administered to a patient.

In yet another embodiment, a physician or clinician may determine the genotypes, at one or more genetic loci, of a group of subjects participating in a clinical trial, wherein the subjects display a disorder, e.g., a cancer or prostatic disorder as described herein, and the clinical trial is designed to test the efficacy of a pharmaceutical composition, e.g., a composition consisting of one or more isoform-specific inhibitors, and optionally a second agent, and wherein the physician or clinician attempts to correlate the genotypes of the subjects with their response to the pharmaceutical composition.

### Methods of Detecting Nucleic Acids Encoding Oncogenic Isoforms Using RT-PCR or PCR

The invention also provides methods of detecting a nucleic acid which encodes an oncogenic isoform provided herein, comprising: (a) obtaining cDNA from mRNA obtained from a suitable sample; (b) amplifying the cDNA corresponding to the proto-oncogene, oncogenic isoform, or an epitope fragment thereof; (c) comparing the amplified cDNA to the DNA of a nucleic acid known to encode proto-oncogene, oncogenic isoform, or epitope fragment thereof, wherein the presence of the oncogenic isoform in the amplified cDNA indicates the detection of a nucleic acid encoding the oncogenic isoform.

The invention also provides methods for detecting a nucleic acid which encodes an oncogenic isoform provided herein, comprising: (a) contacting a suitable sample with a compound capable of specifically binding a nucleic acid encoding oncogenic isoform provided herein; and (b) determining whether any compound is bound to the nucleic acid, where the presence of compound bound to the nucleic acid in the sample indicates the detection of a nucleic acid encoding the oncogenic isoform.

The term “sample,” as it refers to samples used for detecting nucleic acids includes, but is not limited to, cells, cell lysates, nucleic acids extracts of cells, tissue samples, or body fluids. Body fluids include, but are not limited to, blood, serum and saliva. In one embodiment, the suitable sample is obtained from a subject.

Methods of obtaining mRNA from a suitable sample are well known in the art. Further, methods of making cDNA from mRNA, such as reverse transcription, are also well known in the art.

As used herein, “amplifying” means increasing the numbers of copies of a specific DNA fragment. In one embodiment, the amplifying of the cDNA is carried out using PCR (polymerase chain reaction).

In one embodiment, the amplifying of the cDNA is accomplished using primers flanking the entire reading frame of a proto-oncogene encoding an oncoogenic isoform polypeptide. In another embodiment, the amplifying of the cDNA is accomplished out using primers flanking a portion, e.g. an exon, of a nucleic acid encoding the polypeptide oncogenic isoform. In yet another embodiment, one or more of the primers hybridize to sequences of the oncogenic isoform which are present in the nucleic acid encoding the oncogenic isoform, but absent in the nucleic acid encoding a non-oncogenic isoform, or vice versa. In yet another embodiment, a primer may hybridize to a sequence selected from the group consisting of SEQ ID NOs: 1, 3, 5, 7, 9, 11, 13, 15, or 17. In certain embodiments, a primer may be 18-22 nucleotides in length.

In one embodiment, comparing the amplified cDNA to the cDNA of a nucleic acid known to encode the proto-oncogene, oncogenic isoform, or epitope fragment thereof is accomplished by comparing the sequence of the amplified cDNA to the known sequence corresponding to the proto-oncogene, oncogenic isoform, or epitope fragment thereof. The presence or absence of sequence in the amplified sequence will indicate that the oncogenic isoform is present or absent.

In another embodiment, comparing the amplified cDNA to the cDNA of a nucleic acid known to encode the proto-oncogene, oncogenic isoform, or epitope fragment thereof is accomplished by comparing the size of the amplified cDNA to the size of the DNA of a gene known to correspond to the proto-oncogene, oncogenic isoform, or epitope fragment thereof. A difference in size will indicate that the amplified DNA encodes an oncogenic isoform.

The invention also provides methods of determining whether a subject is expressing an oncogenic isoform comprising: (a) obtaining cDNA from mRNA obtained from a suitable sample from the subject; (b) amplifying the cDNA corresponding to the proto-oncogene, oncogenic isoform, or an epitope fragment thereof; and (c) comparing the amplified cDNA to the cDNA of a nucleic acid known to encode the proto-oncogene, oncogenic isoform, or an epitope fragment thereof, wherein the presence of the oncogenic isoform in the amplified cDNA indicates that the subject is expressing the oncogenic isoform.

A “suitable sample” in connection with the above method of determining whether a subject is expressing an oncogenic isoform refers to any sample from the subject that could contain the oncogenic isoform. Examples include, but are not limited to, body fluids and tissue samples. Examples of body fluids include, but are not limited to, blood, serum, urine and saliva.

Amplifying, comparing, and determining the presence of the cDNA may be accomplished as stated above.

### Nucleic Acids

In one embodiment the invention provides isolated nucleic acids encoding the oncogenic polypeptide isoforms provided herein, or a substantially identical sequence thereof.

In one embodiment, the invention also provides isolated nucleic acids encoding the polypeptides of oncogenic isoforms or epitope fragments thereof. In one embodiment, the invention provides isolated nucleic acids encoding polypeptides of human oncogenic isoforms or epitope fragments thereof. In one embodiment, the isolated nucleic acid encodes an isoform or epitope fragment thereof of an oncogenic form of a proto-oncogene is selected from the group consisting of FGFR2, FGFR1, RON Receptor tyrosine kinase, KIT receptor tyrosine kinase, PDGF, and PDGFR-alpha.

In one embodiment, the invention provides isolated nucleic acids encoding rat polypeptides of oncogenic isoforms or epitope fragments thereof. In one embodiment, the invention provides isolated nucleic acids encoding mouse polypeptides of human oncogenic isoforms or epitope fragments thereof. In other embodiments the isolated nucleic acids encoding polypeptides of human oncogenic isoforms or epitope fragments thereof will be derived from other species, including but not limited to, dogs, pigs, guinea pigs and rabbits.

### FGFR2

In one embodiment the invention provides an isolated nucleic acid encoding an oncogenic polypeptide isoform or epitope fragment thereof comprising a segment of nucleotides which arise from an alternative use of Exon III of a nucleic acid encoding a FGFR2. In one embodiment, the alternative use of Exon III results in sequence variation in the region of amino acids from 301-360, when aligned with FGFR2 IIIb. Thus, in one aspect the nucleic acid encodes a polypeptide comprising a sequence selected from the group of SEQ NOs: 2, 4, 6, and 8. In another aspect the nucleic acid comprises a sequence selected from the group consisting of SEQ NOs: 1, 3, 5, and 7.

### FGFR1

In another embodiment, the invention provides an isolated nucleic acid encoding an oncogenic polypeptide isoform or epitope fragment thereof comprising a segment of nucleotides which arise from an alternative deletion of Exons 7 and 8 of FGFR1. In one embodiment, the alternative deletion of Exons 7 and 8 results in a deletion of 105 amino acids, when aligned with an FGFR1 proto-oncogene. Thus, in one aspect the isolated nucleic acid encodes a polypeptide comprising a sequence of SEQ NO: 10. In another aspect, the nucleic acid comprises the sequence of SEQ NO: 9.

### Ron Receptor Tyrosine Kinase

In another embodiment, the invention provides an isolated nucleic acid encoding polypeptides of oncogenic isoforms or epitope fragments thereof comprising a segment of nucleotides which arise from an alternative deletion of Exons 5 and 6 of RON receptor tyrosine kinase. In one embodiment, the alternative deletion of Exons 5 and 6 results in an in-frame deletion of 109 amino acids in the extracellular domain, when aligned with a RON receptor tyrosine kinase proto-oncogene. In one aspect, the isolated nucleic acid comprises a juxtaposition of Exons 4 and 7. Thus, in one aspect the isolated nucleic acid encodes a polypeptide comprising the sequence of SEQ NO: 12. In another aspect the isolated nucleic acid comprises the sequence of SEQ NO: 11.

### KIT Receptor Tyrosine Kinase

In another embodiment, the invention provides an isolated nucleic acid encoding a polypeptide of an oncogenic isoform or epitope fragment thereof comprising a segment of nucleotides which arise from an alternative deletion of Exon 11 of a nucleic acid encoding KIT receptor tyrosine kinase. Thus, in one aspect the isolated nucleic acid encodes a polypeptide comprising the sequence of SEQ NO: 14. In another aspect the nucleic acid comprises the sequence of SEQ NO: 13.

### PDGF

In another embodiment, the invention provides an isolated nucleic acid encoding a polypeptide of an oncogenic isoform or epitope fragment thereof comprising a segment of nucleotides which arise from an alternative in-frame deletion of Exon 6 of PDGF. Thus, in one aspect the isolated nucleic acid encodes a polypeptide comprising the sequence of SEQ NO: 16. In another aspect the isolated nucleic acid comprises the sequence of SEQ NO: 15.

### PDGFR-alpha

In another embodiment, the invention provides an isolated nucleic acid encoding a polypeptide of an oncogenic isoform or epitope fragment thereof comprising a segment of nucleotides which arise from an alternative deletion of Exons 7 and 8 (e.g., amino acids 374-456) of PDGFR-alpha. Thus, in one aspect the isolated nucleic acid encodes a polypeptide comprising the sequence of SEQ NO: 18. In another aspect the nucleic acid comprises the sequence of SEQ NO: 17.

Alternatively, an isolated nucleic acid encoding a polypeptide of an oncogenic isoform or epitope fragment thereof may be encoded by a nucleic acid which is substantially identical to a nucleic acid of an oncogenic isoform or epitope fragment thereof provided herein. Likewise, an isolated nucleic acid may encode a polypeptide of an oncogenic isoform or epitope fragment thereof which is substantially identical to an oncogenic isoform or epitope fragment thereof, as provided herein.

A sequence (polypeptide or nucleic acid) that “substantially corresponds” to another sequence may be a sequence that allows single amino acid or nucleotide substitutions, deletions and/or insertions. In one embodiment, sequences that substantially correspond have 80% sequence identity. In another embodiment, sequences that substantially correspond have 85% sequence identity. In another embodiment, sequences that substantially correspond have 90% sequence identity. In another embodiment, sequences that substantially correspond have 95% sequence identity. In another embodiment, sequences that substantially correspond have 97% sequence identity. In another embodiment, sequences that substantially correspond have 99% sequence identity.

In another embodiment, the nucleic acid encodes an oncogenic isoform or epitope fragment thereof comprising the amino acid sequence set forth in SEQ ID NOs: 2, 4, 6, 8, 10, 12, 14, 16, 18, 19, or 20, but with a conservative amino acid substitution. In another embodiment, the nucleic acid encodes an oncogenic isoform or epitope fragment thereof comprising 1, 2, 3, 4, 5, 6, 7, 8, 9 or 10 conservative amino acid substitutions with respect to SEQ ID NOs: 2, 4, 6, 8, 10, 12, 14, 16, 18, 19, or 20. In another embodiment, the nucleic acid encodes an oncogenic polypeptide insert variant comprising 11, 12, 13, 14, 15, 16, 17, 18, 19, or 20 conservative amino acid substitutions with respect to SEQ ID NOs: 2, 4, 6, 8, 10, 12, 14, 16, 18, 19, or 20.

The invention also provides an isolated nucleic acid that specifically binds to a nucleic acid provided herein or a nucleic acid capable of hybridizing under high stringency conditions to a nucleic acid described herein, or a substantially identical sequence thereof.

The invention provides an isolated nucleic acid capable of hybridizing under high stringency conditions to a nucleic acid encoding an oncogenic isoform or epitope fragment thereof comprising SEQ ID NO: 2, 4, 6, 8, 10, 12, 14, 16, 18, 19, or 20 or a substantially identical sequence thereof. The invention provides an isolated nucleic acid capable of hybridizing under high stringency conditions to a nucleic acid comprising the sequence of SEQ ID NO: 1, 3, 5, 7, 9, 11, 13, 15, or 17, or a fragment thereof.

This invention also provides isolated nucleic acids encoding an oncogenic isoform or epitope fragment thereof, wherein the nucleic acid is at least 80% identical to a nucleic acid encoding an oncogenic isoform or epitope fragment thereof, wherein the nucleic acid encoding the oncogenic isoform or epitope fragment thereof comprises a segment of nucleotides at a position which corresponds to the alternative slice junction which when used renders the polypeptide oncogenic. In increasingly more preferred embodiments, rather than 80%, the percent identity is 85%, 90%, 95%, 97%, or 99%.

The nucleic acids described herein can be labeled with a detectable marker. Detectable markers include, but are not limited to: a radioactive marker, a colorimetric marker, a luminescent marker, an enzyme marker and a fluorescent marker. Radioactive markers include, but are not limited to: ³H, ¹⁴C, ³²P, ³³P, ³⁵S, ³⁶Cl, ⁵¹Cr, ⁵⁷Co, ⁵⁹Co, ⁵⁹Fe, ⁹⁰Y, ¹²⁵I, ¹³¹I, and ¹⁸⁶Re. Fluorescent markers include, but are not limited to, fluorescein, rhodamine and auramine. Colorimetric markers include, but are not limited to, biotin and digoxigenin. Any suitable method for attaching markers to nucleic acids may be used with the nucleotides of the invention, and many such methods are well known in the art.

Further, the invention provides nucleic acids complementary to the nucleic acids disclosed herein. By a nucleic acid sequence “homologous to” or “complementary to”, it is meant a nucleic acid that selectively hybridizes, duplexes or binds to a target nucleic acid sequence. For example, adenine is complementary to thymine as they can form two hydrogen bonds. Similarly, guanine and cytosine are complementary since they can form three hydrogen bonds. A nucleic acid sequence, which is homologous to a target sequence, can include sequences, which are shorter or longer than the target sequence as long as they meet the functional test set forth.

It will be readily understood by those skilled in the art and it is intended here, that when reference is made to particular sequence listings, such reference includes sequences which substantially correspond to its complementary sequence and those described including allowances for minor sequencing errors, single base changes, deletions, substitutions and the like, such that any such sequence variation corresponds to the nucleic acid encoding the polypeptide to which the relevant sequence listing relates.

### Vectors

The invention also provides vectors comprising nucleotides encoding a polypeptide of an oncogenic isoform or epitope thereof provided herein. In one embodiment, the vectors comprise nucleotides encoding a polypeptide of an oncogenic isoform or epitope fragment thereof provided herein. In one embodiment, the vectors comprise the nucleotide sequences described herein. The vectors include, but are not limited to, a virus, plasmid, cosmid, lambda phage or a yeast artificial chromosome (YAC).

In accordance with the invention, numerous vector systems may be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance, (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity. Expression of the gene encoding a polypeptide of an oncogenic isoform or epitope fragment thereof results in production of the polypeptide of an oncogenic isoform or epitope fragment thereof.

Methods and conditions for culturing the resulting transfected cells and for recovering the polypeptide of an oncogenic isoform or epitope fragment thereof so produced are well known to those skilled in the art, and may be varied or optimized depending upon the specific expression vector and mammalian host cell employed, based upon the present description.

### Cells

The invention also provides host cells comprising a nucleic acid encoding a polypeptide of an oncogenic isoform or epitope fragment thereof as described herein.

In one embodiment, the host cells are genetically engineered to comprise nucleic acids encoding a polypeptide of an oncogenic isoform or epitope fragment thereof.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The invention also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

The Examples that follow are set forth to aid in the understanding of the inventions but are not intended to, and should not be construed to, limit its scope in any way.

### EXAMPLES

### Example 1

### Isoform Specific Epitopes

### Example 1.1

### FGFR2: Isoform FGFR2-IIIc (SEQ ID NO: 19)

This isoform of Fibroblast Growth Factor Receptor 2 (FGFR2) is predominantly expressed in hormone-refractory prostate cancer. Alternative usage of exon III results in different sequence in the Ig-like loop III of the extracellular domain, which is critical for ligand binding. Isoform IIIb is expressed in normal prostate epithelial cells. Malignant prostate cancer cells switch to IIIc isoform, which has high binding affinity to growth factors with high transforming activities, e.g., FGF8b isoform.

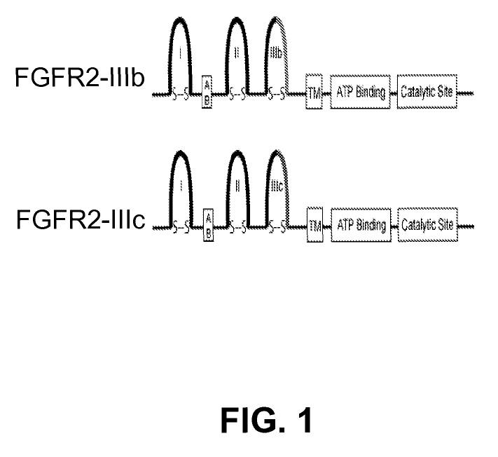

FGFR2-IIIc uses the alternative exon III, which encodes difference sequence than that in isoform FGFR2-IIIb. FGFR2-IIIc isoform contains non-homologous sequence with IIIb isoform in the region of the carboxyl terminal half of the Ig-loop III region, from amino acid position 314 to 353. The isoform structure of FGFR2 is shown in FIG. 1.

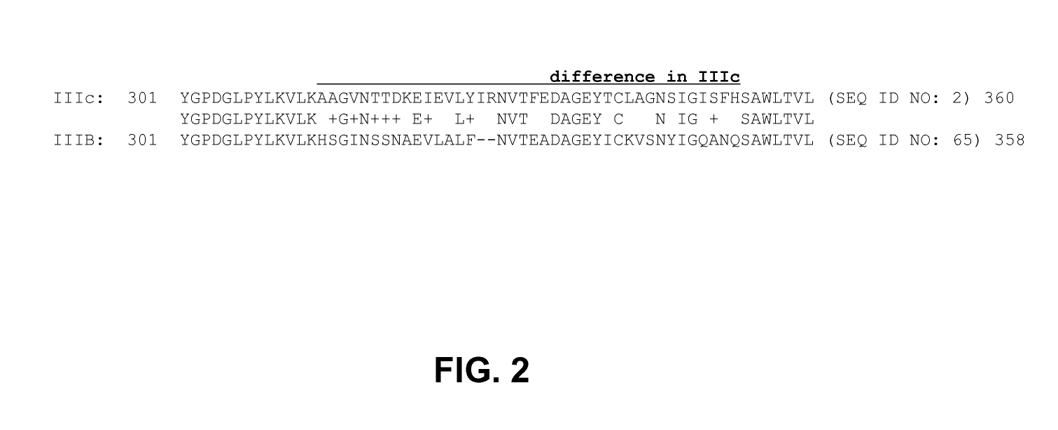

Sequence alignment of IIIc and IIIb isoforms shows the differences in carboxyl terminal half of the Ig loop III region (FIG. 2).

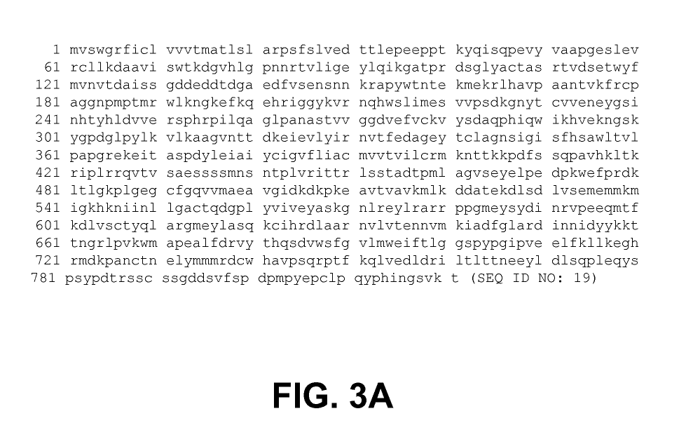

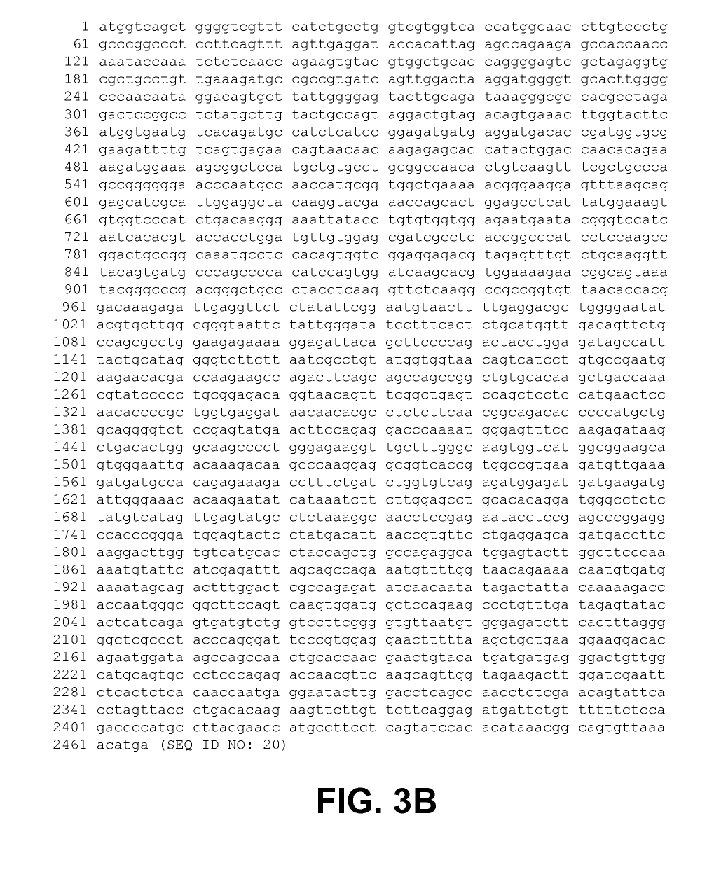

Amino acid (SEQ ID NO: 19) and nucleotide (SEQ ID NO: 20) sequences of FGFR2-IIIc are shown in FIGS. 3A and 3B respectively.

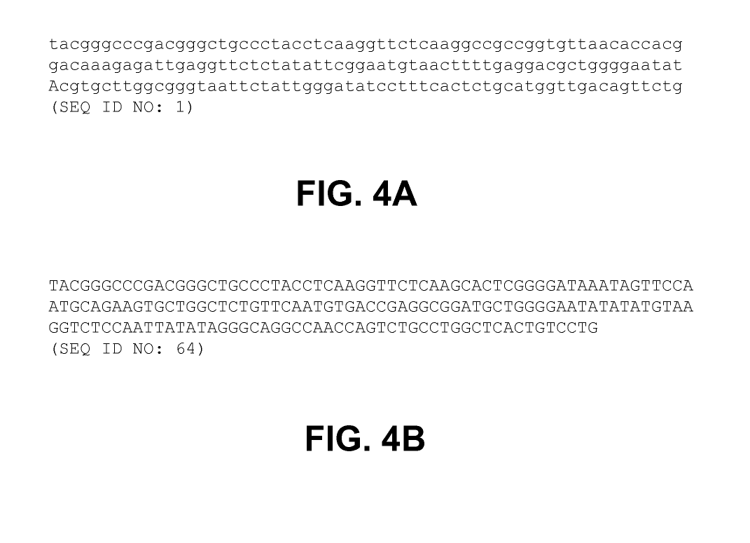

Nucleotide (SEQ ID NO: 1) and amino acid (SEQ ID NO: 2) sequences of FGFR2 Exon-IIIc are shown in FIGS. 4A and 2, respectively. Nucleotide (SEQ ID NO: 64) and amino acid (SEQ ID NO: 65) sequences of FGFR2 Exon-IIIb are shown in FIGS. 4B and 2, respectively.

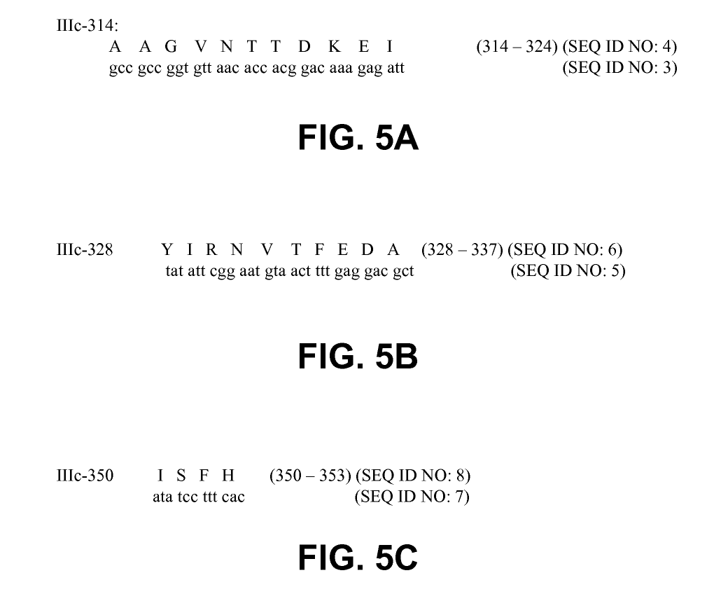

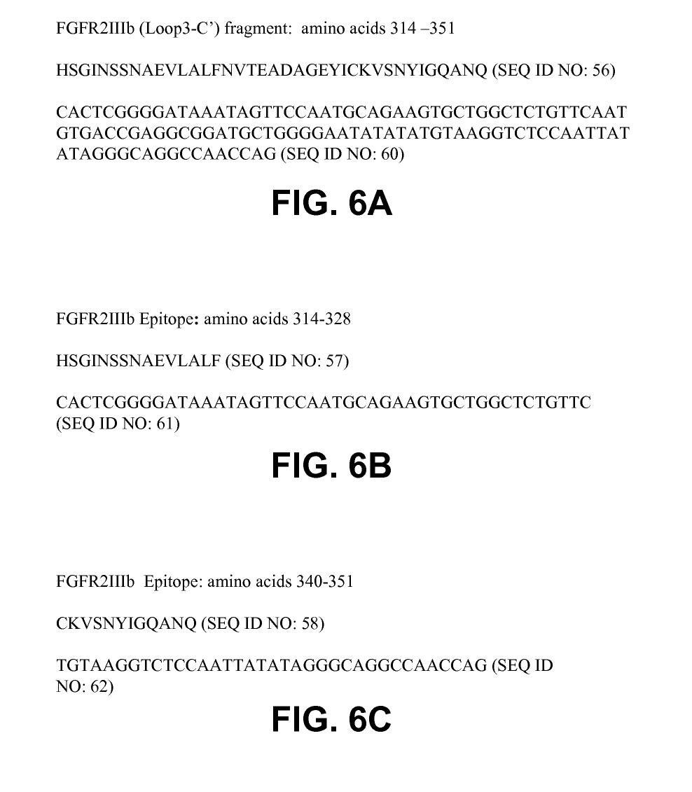

Short peptide sequences were also used as epitopes for generation of monoclonal antibodies. Amino acid (SEQ ID NO: 4) and nucleotide (SEQ ID NO: 3) sequences of IIIc-314, are shown in FIG. 5A. Amino acid (SEQ ID NO: 6) and nucleotide (SEQ ID NO: 5) sequences of IIIc-328 are shown in FIG. 5B. Amino acid (SEQ ID NO: 8) and nucleotide (SEQ ID NO: 7) sequences of IIIc-350 are shown in FIG. 5C. Amino acid (SEQ ID NO: 56) and nucleotide (SEQ ID NO: 60) sequences of IIIb (Loop3-C′) fragment: amino acids 314-351, are shown in FIG. 6A. Amino acid (SEQ ID NO: 57) and nucleotide (SEQ ID NO: 61) sequences of IIIb epitope: amino acids 314-328 are shown in FIG. 6B. Amino acid (SEQ ID NO: 58) and nucleotide (SEQ ID NO: 62) sequences of IIIb epitope: amino acids 340-351 are shown in FIG. 6C.

### Example 1.2

### FGFR1: Isoform FGFR1L (Deletion of Exon 7 & 8; 105 Amino Acids; Part of Ig-II and Part of Ig-III)

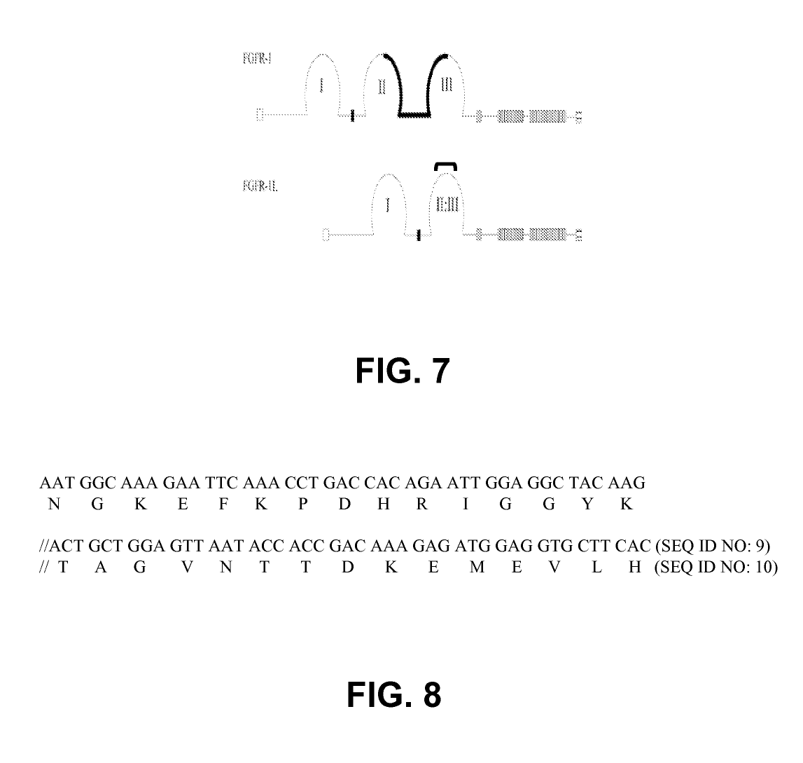

The isoform structure of Fibroblast Growth Factor Receptor 1 (FGFR1) is shown in FIG. 7. The amino acid (SEQ ID NO: 10) and nucleotide (SEQ ID NO: 9) sequences for the epitope at the junction are shown in FIG. 8.

### Example 1.3

### RON Receptor Tyrosine Kinase: Isoform RONΔ160

This isoform of Macrophage stimulating 1 receptor (RON) is constitutively active. Skipping of exons 5 and 6 results in an in-frame deletion of 109 amino acids in the extracellular domain.

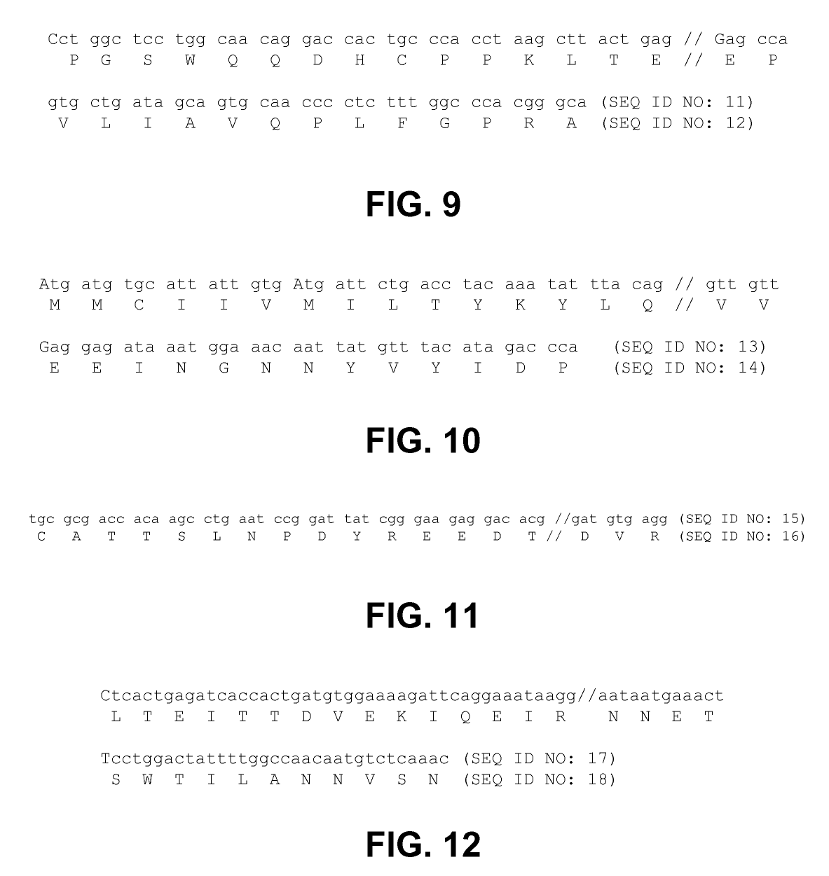

The epitope is at the junction between exon 4 and exon 7. The nucleotide (SEQ ID NO: 11) and amino acid (SEQ ID NO: 12) sequences of this epitope are shown in FIG. 9.

### Example 1.4

### KIT Receptor Tyrosine Kinase (Deletion in Exon 11)

Most gastrointestinal stromal tumors, GISTs, harbor oncogenic mutations in the v-kit Hardy-Zuckerman 4 feline sarcoma viral oncogene homolog (KIT) gene, and the majority of these mutations affect the juxtamembrane domain of the kinase encoded by exon 11.

The nucleotide (SEQ ID NO: 13) and amino acid (SEQ ID NO: 14) sequences for this epitope are shown in FIG. 10.

### Example 1.5

### PDGF: Isoform 2 (In-Frame Deletion of Exon 6)

Platelet-Derived Growth Factor (PDGF) isoform 2 has in-frame deletion of exon 6. The nucleotide (SEQ ID NO: 15) and amino acid (SEQ ID NO: 16) sequences for this epitope are shown in FIG. 11.

### Example 1.6

### PDGFR-alpha: Delta-exon 7 and 8 (amino acids 374 to 456)

Platelet-Derived Growth Factor Receptor alpha (PDGFR-alpha) has deletion in exons 7 and 8. The nucleotide (SEQ ID NO: 17) and amino acid (SEQ ID NO: 18) sequences for this epitope are shown in FIG. 12.

TABLE 1
Sequences used for designing epitopes for isoform-specific antibodies:

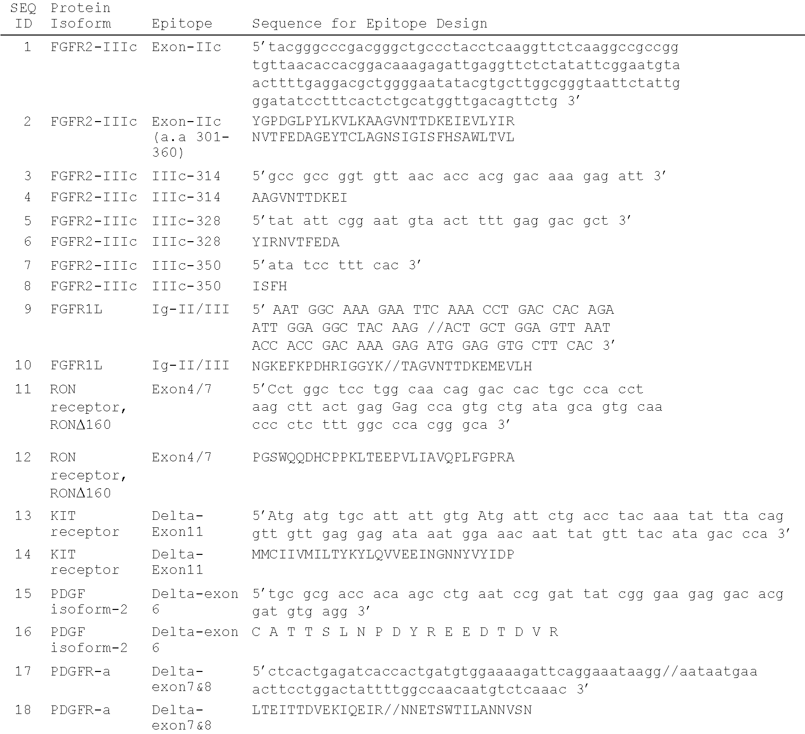

Shaded area = nucleotide seq
Clear area = amino acid seq

### Example 2

### Generation of FGFR2 Isoform Specific Antibody

Antibodies to FGFR2 (non-specific to the isoforms) are commercially available. However, these antibodies do not significantly distinguish between different isoforms. For the purpose of studying the isoform protein distribution and function in tumor and normal tissues, antibodies recognizing isoform-specific sequences for FGFR2-IIIc and IIIb (FIG. 1) were designed. Monoclonal antibodies were generated by common hybridoma technology. Briefly, coding sequences were either PCR amplified or chemically synthesized based on gene sequences of SEQ ID NOs: 19 and 63, respectively. The DNA fragments were subsequently cloned into a commercially available mammalian expression vector. The expression vectors were used for genetic immunization of 5 mice for each antigen. Immunized mice that had serum titer greater than 40.000-fold by ELISA test were used for fusion with myeloma SP 2/0 cells for generation of hybridoma clones.

Monoclonal antibodies were screened by ELISA and Western blots for affinity and specificity. Multiple monoclonal antibody clones for each isoform were further characterized by binding specificity, affinity and IC₅₀ (concentration at 50% inhibition) against target receptors. Receptors were prepared either as full-length membrane bound receptor (for cell-based assays) or as soluble form of the extracellular domain fused to human IgG Fc (for ELISA based tests). Positive monoclonal antibody clones to FGFR2IIIc were chosen for further development based on the following criteria (i) no detectable cross-reactivity with FGFR2IIIb isoform, (ii) nanomolar affinity to its receptor based on EC₅₀ value, (iii) staining profile in prostate tumor, other tumors and normal tissue controls. Anti-FGFR2IIIb monoclonal antibody clones were chosen by similar criteria and used as a control for in vitro studies and for IHC staining of normal and tumor tissues.

These monoclonal antibodies can be humanized by using routine procedures. For example, humanized anti-FGFR2 isoform specific antibodies can be generated by replacing sequences of the Fv variable region which are not directly involved in antigen binding with equivalent sequences from human Fv variable regions, as described by, e.g., Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Humanized anti-FGFR2 isoform specific antibodies can also be produced by CDR-grafting or CDR substitution, wherein one, two, or all CDRs of an immunoglobulin chain are replaced, as described in, e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference.

The anti-FGFR2 isoform specific antibodies can also be produced by phage display technology. Phage display techniques for generating anti-FGFR2 isoform specific antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

### Example 3

### Generation of Soluble FGFR2 IIIc-Fc Receptor

A DNA sequence encoding the extracellular domain of the human FGFR2beta(IIIc) protein (nucleotides 1-786 of SEQ ID NO: 54) was fused to the carboxy-terminal Fc region of human IgG1. The two gene fragments were jointed by a 6 nucleotide linker from a restriction enzyme (Bgl-II), which created two amino acid residues, Arginine and Serine. The total sequence encodes a polypeptide of 491 amino acids. The signal sequence is 21 amino acids; therefore the mature protein of this chimera is 470 amino acids in length. The calculated molecular weight is 52.81 kilodaltons (kDa).

The structure of this fusion protein is illustrated in FIG. 13A.

The nucleotide (SEQ ID NO: 54) and amino acid (SEQ ID NO: 55) sequences of the soluble FGFR2 IIIc-Fc fusion protein are shown in FIGS. 13B and 13C, respectively.

The fusion protein was expressed by generation of stable cell lines in CHO host cells.

The recombinant protein is soluble and secreted in the culture media. By analysis on SDS-PAGE under reduced conditions, the recombinant protein migrates as an approximately 95 kDa protein, presumably as a result of glycosylation (FIG. 13D). In FIG. 13D, lane 7 shows the molecular weight standards. Thirteen clonal cell lines were analyzed on the blot (lanes 1-6, 8-14). The conditioned media (20 microliter per lane) from each clone was run on the SDS-PAGE gel, subsequently transferred onto Western blot. The blot was stained with a secondary antibody, goat-anti-human IgG conjugated with alkaline phosphatase. Lane 3, 10, 11 and 14 show positive expression of the Fc fusion protein of FGFR2 beta-ECD from stable clone number 1D2, 1F5, 1F7 and 1F10.

### Example 4

### Generation of FGFR2 IIIc Peptide

Isoform-specific peptides of FGFR2 IIc can be generated by standard recombinant or solid phase synthesis.

For example, peptides having the amino acid sequences shown in Table 1 and FIGS. 6A-6C can be generated by cloning the corresponding nucleotide sequences into an expression vector as described in Example 2.

Alternatively, peptides can be synthesized by standard methods of solid or solution phase peptide chemistry. A summary of the solid phase techniques can be found in Stewart and Young (1963) Solid Phase Peptide Synthesis, W. H. Freeman Co. (San Francisco), and Meienhofer (1973) Hormonal Proteins and Peptides, Academic Press (New York). For classical solution synthesis see Schroder and Lupke, The Peptides, Vol. 1, Academic Press (New York). In general, one or more amino acids or suitably protected amino acids can be sequentially added to a growing peptide chain. The protected amino acid is then either attached to an inert solid support or utilized in solution by adding the next amino acid in the sequence having the complimentary (amino or carboxyl) group suitably protected and under conditions suitable for forming the amide linkage. The protecting group is then removed from this newly added amino acid residue and the next amino acid (suitably protected) is added, and so forth. After all the desired amino acids have been linked in the proper sequence, any remaining protecting groups (and any solid support) are removed sequentially or concurrently to afford the final peptide. More than one amino acid can be added at a time to a growing chain, for example, by coupling (under conditions which do not racemize chiral centers) a protected tripeptide with a properly protected dipeptide to form, after deprotection, a pentapeptide.

### Example 5

### Testing of Antibody Molecules for Targeting of Fibroblast Growth Factor Receptor-2 (FGFR2) Isoform IIIc in Prostate Cancer

This example evaluates the therapeutic feasibility of an antibody drug against FGFR2 IIc (anti-FGFR2-IIIc antibody) in prostate cancer models. The molecular target of the antibody, FGFR2 isoform Inc, has been associated with androgen-independent tumor growth and metastasis. This approach is based on the high expression level of this receptor on hormone-refractory prostate cancer (HRPC) and its key role in enhancing the invasive behavior of tumor cells (epithelial-to-mesenchymal transition, EMT). This isoform-specific antibody drug is designed with the intention of targeting the “bad isoform” of FGFR2 receptor on tumor, but spare the “good isoform” FGFR2-IIIb on normal prostate epithelium that functions to suppress tumor growth.

### Cell Lines

The androgen-independent human prostate cancer cells DU145 (ATCC) and DU9479 (Duke University) used in this study are both well-characterized cell lines displaying metastatic properties and androgen-independent growth. DU145 was derived from carcinoma of prostate cancer metastasized to the brain (Stone et al. (1978) Int. J. Cancer 21: 274-281). This cell line expresses predominantly FGFR2-IIIc (Carstens et al., (1997) Oncogene 15, 3059-3065). It has been frequently used in animal model studies for tumor growth and angiogenesis (Garrison et al., (2007) Cancer Res. 67:11344-11352; Russel and Voeks (2003) Methods in Molecular Medicine™: “Prostate Cancer: Methods and Protocols” Animal Models of Prostate Cancer. Page 89-112). Other human prostate tumor lines, e.g. PC-3 (hormone-independent) or LNCaP (hormone dependent, express IIIb) are used for some work as comparisons or negative controls. DU9479 is another androgen insensitive line, and consists of entirely FGFR2IIIc isoform (Carstens et al., (1997) Oncogene 15, 3059-3065).

For in vitro cell based assays, DU145 is suitable for most of the experiments, including proliferation and receptor activation assays. For the ligand binding assay, Transfected cells that express the recombinant FGFR2IIIc target can be used.

Monoclonal antibodies anti-FGFR2-IIIc and IIIb were generated and characterized (referenced herein as Ab-1 and Ab-2, respectively). Other biochemical reagents (FGFs) and immunochemical reagents (e.g. antibodies to phosphotyrosine, signaling molecules of Grb2, ERK1/2, STAT1 and SHP2) can be purchased from various commercial vendors.

The antibody molecules can be tested in vitro and in vivo using hormone-independent tumor lines, DU145 and DU9479. The following experiments can be conducted:

### 1) Testing Ab-1 In Vitro Activity and Cellular Mechanism

a. Inhibition of receptor activation and signaling

b. Blocking ligand binding or receptor dimerization

c. Effects on cell proliferation and apoptosis

### 2) Testing Ab-1 In Vivo Efficacy in Human Prostate Cancer Xenografts

d. Effect on inhibiting tumor growth, tumor angiogenesis

Monoclonal antibody Ab-1 was developed with a dual functionality in the design. The mode-of-action for this antibody can include both inhibitor function, i.e. blocking receptor's activation, and immunological function, i.e. inducing cytotoxic T-cell activity. Ab-1 binds to the isoform-specific domain of Ig-like loop-3 on the FGFR2IIIc receptor. This domain is involved in ligand binding specificity as previously demonstrated by crystal structure analysis (Shaun et al., (2006) Genes & Dev. 20: 185-198). It is expected that antibody Ab-1 can block ligand binding, therefore, inhibiting receptor activation. Secondly, the antibody can activate the body's cellular immunity. This antibody is a human IgG1 isotype and can elicit strong immune responses of antibody-dependent cellular cytotoxicity (ADCC) and/or complement-mediated cell lysis. Ab-1 is engineered at amino acid position 333 from glutamine to alanine in the Fc region to further enhance the ADCC activity of the antibody. Therefore, when the antibody binds to FGFR2IIIc positive tumor cells, it can recruit cytotoxic T-cells via ADCC to mount potent tumor killing activities.

Thus, Ab-1 can have robust anti-tumor activity, and at the same time, can have an attractive safety feature. Because it binds strictly to the IIIc-positive tumor, it can selectively kill tumor cells without causing serious side-effects to normal epithelial tissues (which express IIIb isoform).

### Example 5.1

### Validation Studies for Expression of FGFR2IIIc

Validation studies for expression of FGFR2IIIc in a broad range of cancer cell lines, including prostate, bladder, lung (NSCLC) and thyroid, were performed. FGFR2IIIc expression was also investigated by tissue-distribution profiling (IHC staining). The following studies were conducted.
Demonstrate specific binding to prostate tumor cells, not by matched normal prostate (Tissue Arrays compliant with FDA, from US Biomax, Rockville, Md. 20849; Multi-Tumor Microarrays from Invitrogen)
IHC staining for 30 organ tissue arrays to demonstrate no cross reactivity to healthy tissue (Tissue Arrays from US Biomax, Rockville, Md. 20849)

### Example 5.2

### Construction of FGF8-SEAP

This construct was made to facilitate a sensitive, non-labeling ligand-binding assay. The coding sequence of FGF8b was PCR cloned from cDNA template (SEQ ID NO: 66) and inserted behind the secreted alkaline phosphatase gene in a commercially available expression vector. A flexible linker of 10-amino acid GGGGSGGGGS (SEQ ID NO: 59) was added between the two fragments, and a His-tag was added to the C-terminal of the fusion protein to facilitate protein purification. The resulting fusion protein, FGF8-SEAP can be easily prepared as secreted form in cell supernatant and used directly for most of the assays. To quantify the enzymatic activity, purified SEAP (commercially available) was used as a standard, and chemiluminescent substrate was used for measuring the light signal. SEAP activity directly correlates with the quantity of the ligand FGF8b.

### Example 5.3

### In Vitro Studies for Cellular Mechanisms

Established anti-cancer antibody drugs, such as Herceptin (anti-Her2 receptor for breast cancer) and Erbitux (anti-EGFR receptor for head and neck cancer) exhibit their anti-tumor activities via diverse mechanisms. These mechanisms include blocking receptor signaling, interfering with ligand-receptor binding, triggering apoptosis, and inducing cytotoxic effects via ADCC or complement-mediated lysis (Baselga et al., (2001) Semin Oncol. 5 Suppl 16:4-11; Trauth et al. (1989) Science 245:301; Yang et al. (1999) Cancer Res. 59:1236). In this case, several in vitro experiments can be used to investigate the anti-tumor activity of Ab-1 to prostate cancer cells, with the intention to provide information for understanding drug's cellular mechanism in prostate cancer cells.

To provide evidence for understanding the cellular mechanism of antibody's action on tumor cells, three aspects of the cellular function can be examined.

a. Effect of Antibody Ab-1 in Blocking FGF Signaling

Receptor Activation Assay—Dose Dependent Inhibition: The neutralizing activity of antibody on FGFR2 receptor activation in DU145 cells can be examined. DU145 is known to express FGFR1, FGFR2IIIc (predominantly) and FGFR4 (Coombes et al., (2000) Book “Endocrine Oncology”, Chapter 12, 237-253; Carstens et al., (1997) Oncogene 15, 3059-3065). FGFR2IIIc binds and responds to FGF8 and FGF2, whereas isoform IIIb receptor does not respond to those two growth factors (Zhang et al., (2006) J Biol. Chem. 281: 15694-15700). Receptor activation can be analyzed as increased phosphorylation by Western blot analysis on cell lysate. In some cases, it is necessary to “pull down” the receptors from total cell lysate by immunoprecipitation with the anti-receptor FGFR2IIIc. The resulting immunoprecipitates are analyzed on SDS-gel, followed by Western blotting using an anti-phosphotyrosine antibody.

To obtain a dose dependent inhibition curve for IC₅₀ value, DU145 cells are incubated with or without increasing concentrations of antibody before challenging with FGF8. The range of antibody concentration can be empirically determined, which is dictated by antibody affinity and receptor expression level on the particular cells. Antibody's inhibition curve can be established via quantification of phosphorylated receptors (e.g. densitometry scan), thus an IC₅₀ value for antibody inhibition of receptor activation can be deduced through these analyses.

In addition, downstream signaling events can be examined by analyzing the signaling molecules or effectors of FGFR2, e.g. Grb2, ERK1/2, p38 or STAT1. These additional readouts can be used to confirm the data. Together with receptor activation, phosphorylation analyses, these results provide information for the potential potency of the drug.

These data can demonstrate whether antibody Ab-1 has neutralizing activity. Mechanistically, the antibody could compete with ligand binding to the receptor, or it could block receptor dimerization. Both can give the same readout as inhibition of receptor activation and signaling. The following experiments are designed to answer those questions.

b. Effect of Blocking Ligand Binding or Receptor Dimerization

Previously reported FGFR2 crystal structure analysis (Olsen et al., (2006) Genes & Dev. 20: 185-198) indicated that the C′-terminal half of the loop-3, which is encoded by the alternative exon 8, is involved in ligand binding specificity of the receptor. Loop-3 in IIIc isoform binds to FGF8, whereas loop-3 of IIIb binds to FGF7. However, it has also been reported that loop-2 of the receptor may also contribute to ligand binding. Therefore, it is necessary to obtain direct evidence through the experiments to demonstrate whether Ab-1, by binding to its epitope in C′-terminal half of the loop-3, can completely block ligand FGF8 binding to its receptor FGFR2IIIc. The assay for antibody inhibition of ligand binding can be performed as below.

Separately, another effect—whether antibody binding to receptor can interfere with receptor dimerization, a prerequisite step for receptor activation and signaling, can be tested. Together, these molecular interaction analyses can provide a detailed understanding of the molecular mechanism of antibody's mode-of-action.

### Ligand Binding Assay:

To assess antibody inhibition on ligand binding, transfected HEK293 cells expressing the receptor FGFR2-IIIc can be used in a 96-well plate assay. Non-radioactive and sensitive luminescence assays to measure ligand binding to its receptor were developed. This assay format involves using a recombinant FGF8 infused with secreted alkaline phosphatase, FGF8-SEAP (as described above). This assay format allows instant enzymatic readout for ligand-receptor binding event via a robust luminescent signal. FGF8-SEAP can be used in the 20 pM to 5 nM concentration range according to previously reported ligand binding conditions (Zhang et al., (2006) J Biol. Chem. 281: 15694-15700). Heparin is added at a concentration of 10 μg/ml to facilitate FGF8 binding to the receptor. The receptor bound ligand can be directly quantified by adding chemiluminescence substrate of SEAP (CDP-Star® from Applied Biosystems, or PhosphaGLO™ from KPL), and measured in a microplate reader (Luminoskan, Thermo Scientific).

The IC₅₀ value can be obtained from a competition experiment, in which antibody Ab-1 is pre-incubated with cells at a concentration range from 1 μM to 100 nM. Subsequently, ligand SEAP-FGF8 is added to the cell culture. Dose dependent reduction of SEAP signal means that antibody competes with ligand binding site on the receptor.

Statistics—Binding curves can be analyzed by fitting sigmoid curves with variable slope using nonlinear regression. Group data are reported as mean+/−SD or SEM.

### Receptor Dimerization Assay:

It is known that FGFs bind to their receptors to induce receptor dimerization. This can be demonstrated using chemical cross-linking reagent, such as cross-linker SDP (succinimidylpropionate). Monomer and dimer receptors are distinguished based on apparent molecular weights on a non-reducing SDS-gel, followed by Western blotting. Receptor from un-treated cells should exist as a monomer (92 Kda). FGF8 treated cells should display predominantly dimmers (−180 Kda).

To examine whether antibody Ab-1 can block receptor dimerization, transfected cells expressing FGFR2IIIc (in 6-well culture plate) are pre-incubated with antibody at 0, EC₅₀ and saturation concentrations. An irrelevant antibody can be used as a negative control. After antibody pre-incubation, FGF8 is added to the cells to induce receptor dimerization. Chemical cross-linker DSP is then added to the cells for an additional incubation of 10 to 15 minutes at room temperature. Finally, cell lysate is prepared and analyzed on Western blot with an anti-receptor antibody. The blot reveals primarily receptor monomers in un-stimulated cells, and increased dimers in FGF8 stimulated cells (in the absence of Ab-1 treatment). Pre-incubation with negative control antibody does not reduce the amount of dimer in FGF8 stimulated sample. Ab-1 treated cells are compared with cells treat with negative control antibody for any reduction of dimers after FGF8 induction. This data provide evidence for whether Ab-1 antibody can block receptor dimer formation.

c. Effect of Ab-1 in Cell Proliferation and Apoptosis:

The anti-proliferative effect of antibody Ab-1 can be evaluated. In addition, the pro-apoptosis effect of antibody Ab-1 on tumor cells can also be analyzed.

### Proliferation Assay:

Several cell lines from prostate cancer, including PC-3, DU145 and LNCaP, can be analyzed in 96-well plates using MTT assay as previously described (Mosmann et al., (1983) J. Immunol. Methods, 65:55-63). MTT provides a measure of mitochondrial dehydrogenase activity within the cell therefore offers an indication of cellular proliferation status.

Cells at exponential growth can be seeded at 2000-3000 cell density in the wells of 96-well plates. AB-1 at nM range is added to the wells with culture medium and incubate for 48 hours. MTT (1 mg/ml) is added to the cells for incubation of 2 hours at 37 C. Cells are lysed, and absorbance of the dye measured in micro-plate reader at 600 nm

### Assessment of Apoptosis:

The effect of antibody Ab-1 on induction of apoptosis in tumor cells can be examined using the lipophilic dye MC540 in combination of DNA-staining dye Hoechest 33342 as previously described procedures (Reid et al., (1996) J Immunol Methods, 192:43-54). MC540 detects early stage of apoptosis (i.e. conformational changes in the plasma membrane). Tumor cells are treated with antibody similarly as described above for proliferation assay. The membrane change is measured by incorporation of the dye MC540. To further assess biochemical alteration in apoptotic cells, Applicants examine the expression of the active form of caspase-7 by Western blotting. Anti-caspase-7 can be purchased from Cell Signaling Technology.

### Example 5.4

### In Vivo Study for Ab-1 Effect on Human Tumor Xenografts

In vivo efficacy for Ab-1 in hormone-independent tumor can be examined in nude mice with DU145 implants. Endpoints include tumor volume, weight, tumor vasculature and metastasis index. Additional readouts, e.g. survival time, immunological responses, and toxicology can also be analyzed.

d. Effect on Blocking Tumor Growth and/or Tumor Angiogenesis in Xenografts

Mice participating in experiments are checked every 2 days for signs of toxicity and discomfort including weight, level of activity, skin abnormalities, diarrhea, and general appearance.

A well-established subcutaneous (s.c.) tumor xenograft model using DU145 prostate cancer cells (Coombes et al., Book “Endocrine Oncology”, Edited by Stephen P. Ethier. Chapter 12, 237-253) can be adapted. Briefly, 5×10⁶ tumor cells are inoculated into 6-week-old nude mice and allowed tumor to grow to 1 cm³ (3-4 weeks for DU145). Tumor fragments of 100 mm³ volume are implanted into mice. Tumor growth is monitored every 3-days by external measurements with a caliper. Tumor-bearing mice are divided into 3 groups of 10 mice. Group-1 can be treated with taxol as positive control group. Group-2 can be treated with antibody AB-1 at 10 mg/kg, 2 times a week, i.p. injection for 5 weeks. Group-3 can be treated with vehicle as a negative control group. Tumor growth is monitored by external measurement. Heparinized blood samples are drawn from the retro-orbital plexus for determination of plasma Ab-1 concentrations.

At the end of the experiments, tumors are excised, weighed, and fixed in formalin. The following endpoint data are collected:
1. Tumor wet weight (grams)
2. Metastasis in secondary sites—lymph node, lung, pancreas, spleen, kidney, adrenal, diaphragm, bone and brain
3. Immunohistochemical staining analysis on fixed specimens for target FGFR2IIIc expression on tumor, FGFR2IIIc activation/phosphorylation, and accumulation of AB-1 on tumors (using anti-human antibody staining by IHC method)
4. Vascularity evaluation using anti-CD31 staining (Dako). Positive endothelial cells will be counted in five different fields

Statistical Analysis: Tumor volume is calculated as V=(L²/l)/2, where L and l represent the larger and the smaller tumor diameter. Endpoint measurement for tumor is wet weight in grams Statistical comparisons are performed using ANOVA for analysis of significance between different values. Regression analysis for caliper volume and wet weight are performed. Group data are reported as mean+/−SD or SEM. P values<0.005 were considered significant.

### Example 5.5

### Alternative Strategies

a. Xenograft Studies:

For metastatic HRPC, complex mechanisms and multiple steps are involved in disease progression. Critical steps of the disease mechanisms involving FGFR2IIIc can be explored using Ab-1, and Ab-1's anti-tumor activity can be demonstrated in a well-established xenograft model. This study can be used to ascertain the activity of the monoclonal antibody in a tumor model. Additional studies may require using different tumor inoculation methods such as orthotopic inoculation or intracardiac injection, in order to dissect the major stages of tumor metastasis.

Besides DU145 xenograph, other CaP tumor lines, which have high expression of the targeted receptor, can also be used.

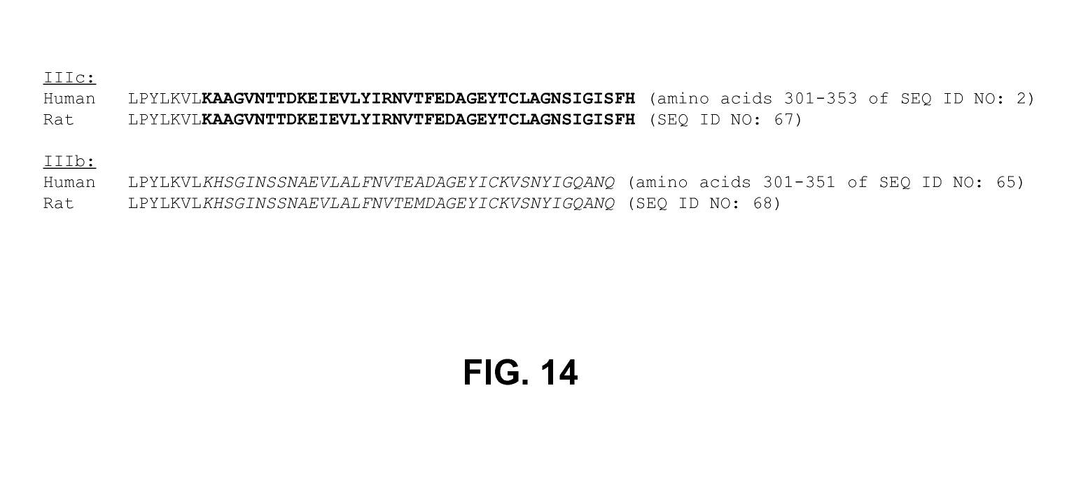

Alternatively, Dunning rat prostate cancer model and the AT-3 hormone-independent cell line can be used. This model system has been used extensively for studying the FGFR2 isoform function/regulation and is considered relevant to human HRPC (Sebastian et al., (2006) PNAS 103:14116-14121; Muh et al., (2002) JBC 277:50143-50154; Carstens et al., (2000) MCB, 20:7388-7400). This approach can be evaluated to confirm that monoclonal antibodies, Ab-1 and Ab-2 (anti-FGFR2IIIc and anti-FGFR2IIIb, respectively) cross-react with rat receptors. The amino acid sequences in the alternatively spliced regions of both IIIc (Human: amino acids 301-353 of SEQ ID NO: 2; Rat: SEQ ID NO: 67) and IIIb (Human: amino acids 301-351 of SEQ ID NO: 65; Rat: SEQ ID NO: 68) are completely conserved between human and rat (FIG. 14).

b. In Vitro Study

Besides DU145 cells, transfected cells with low endogenous FGFR2IIIc expression can be used in the in vitro study.

### Example 5.6

### Other Experiments

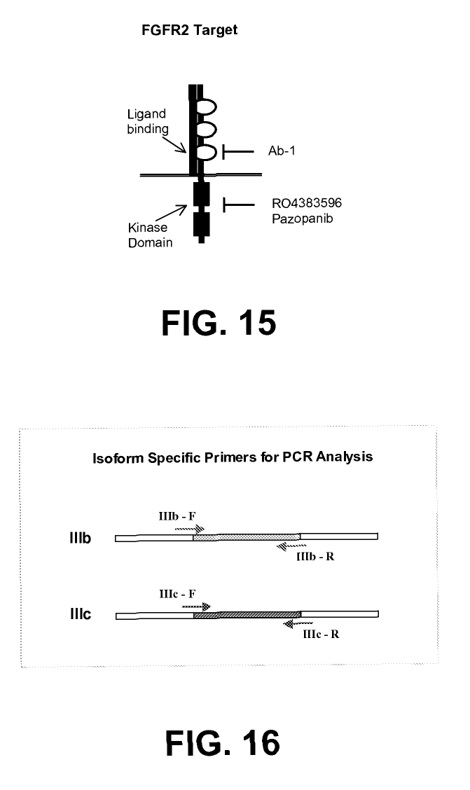

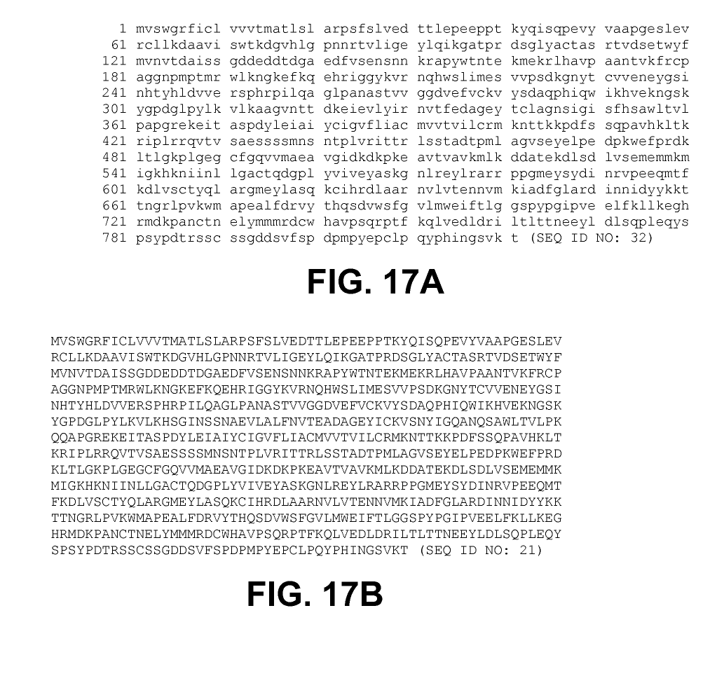

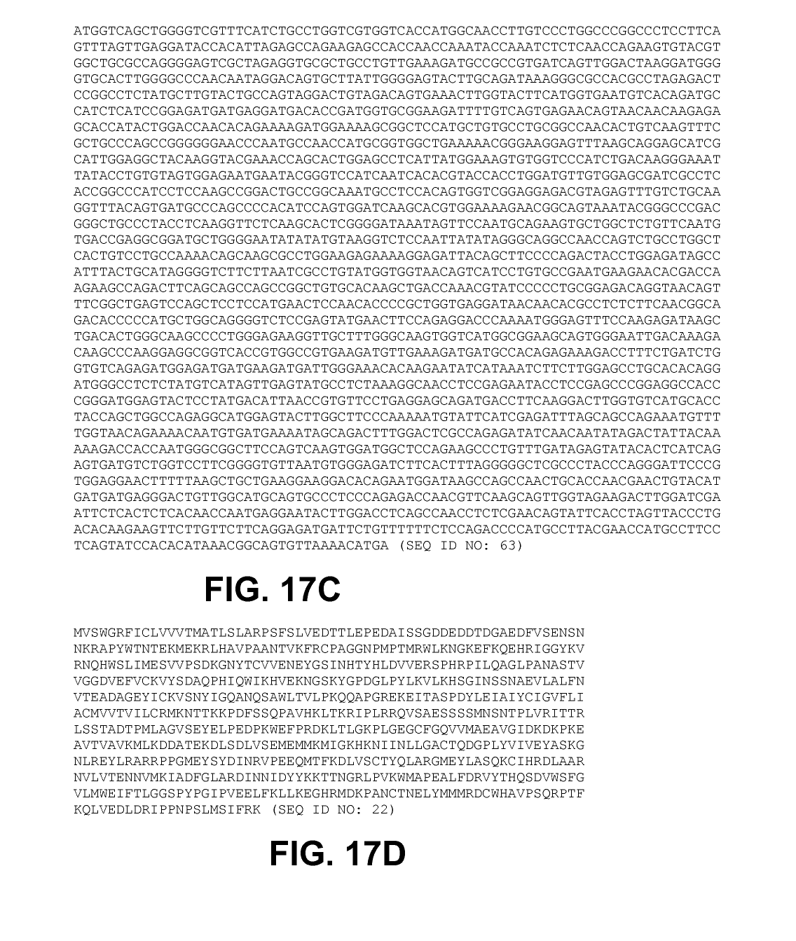

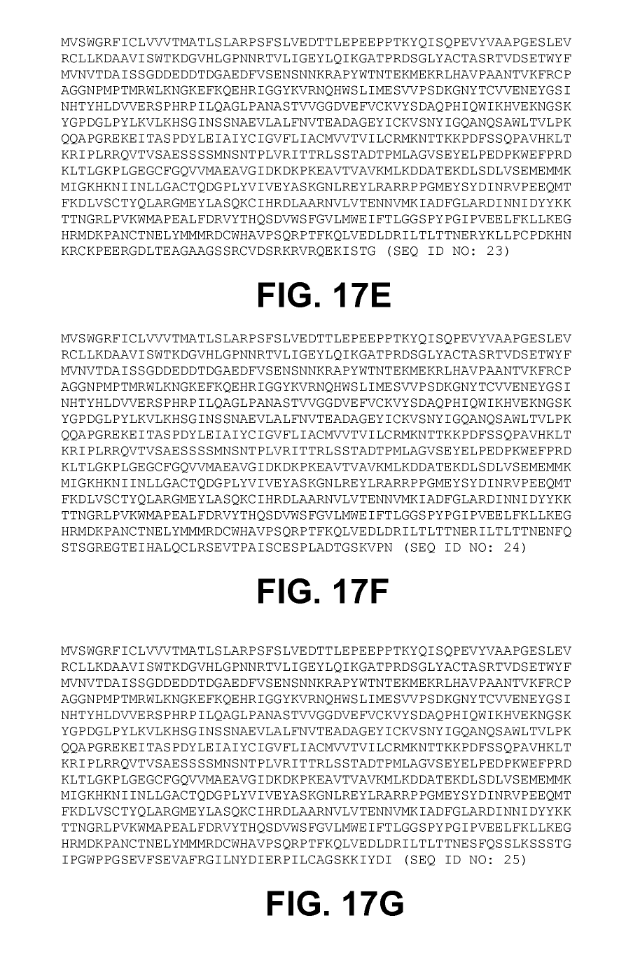

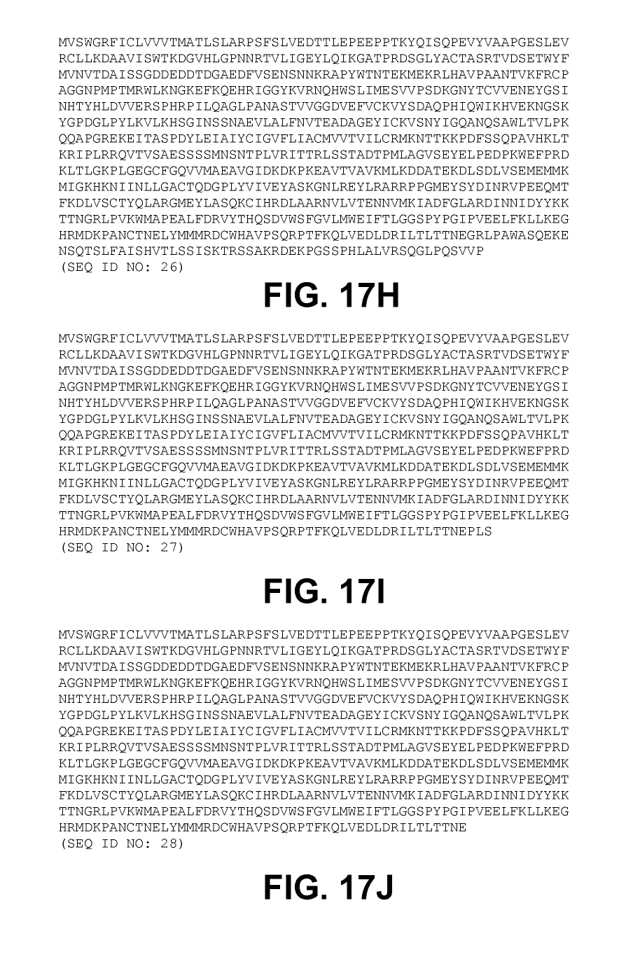

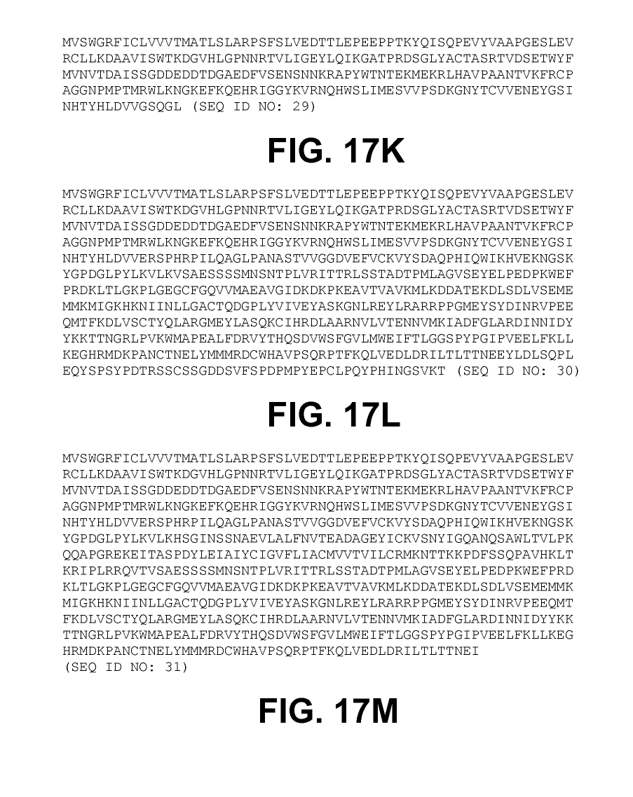

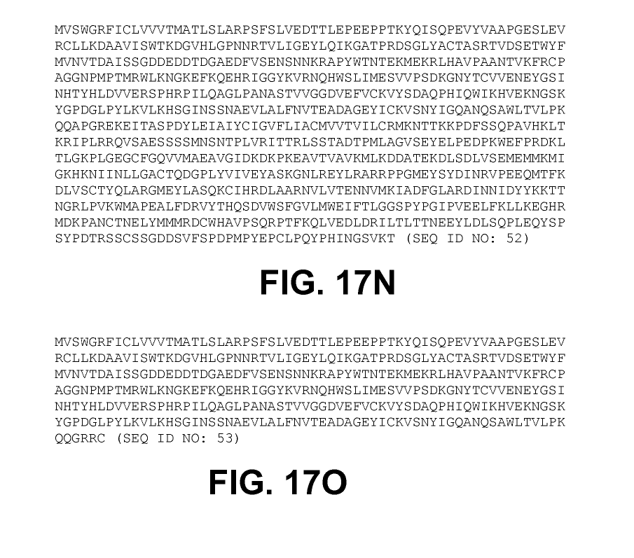

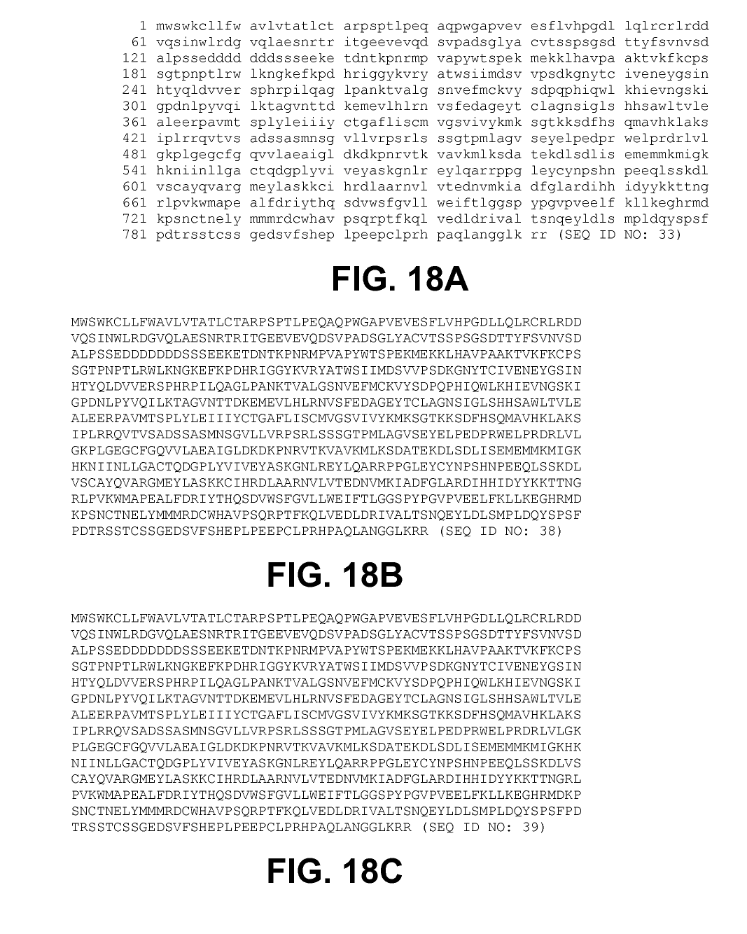

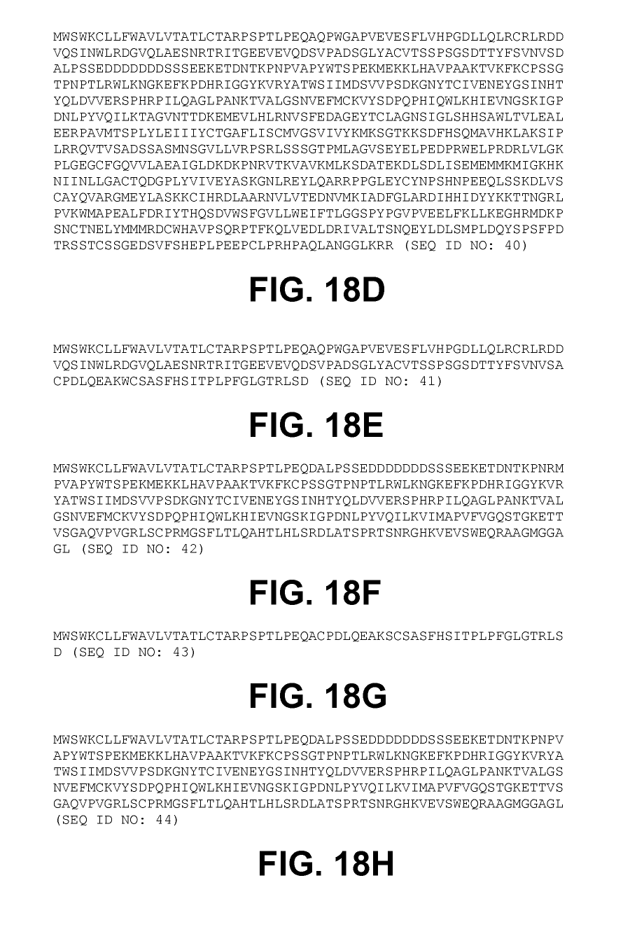

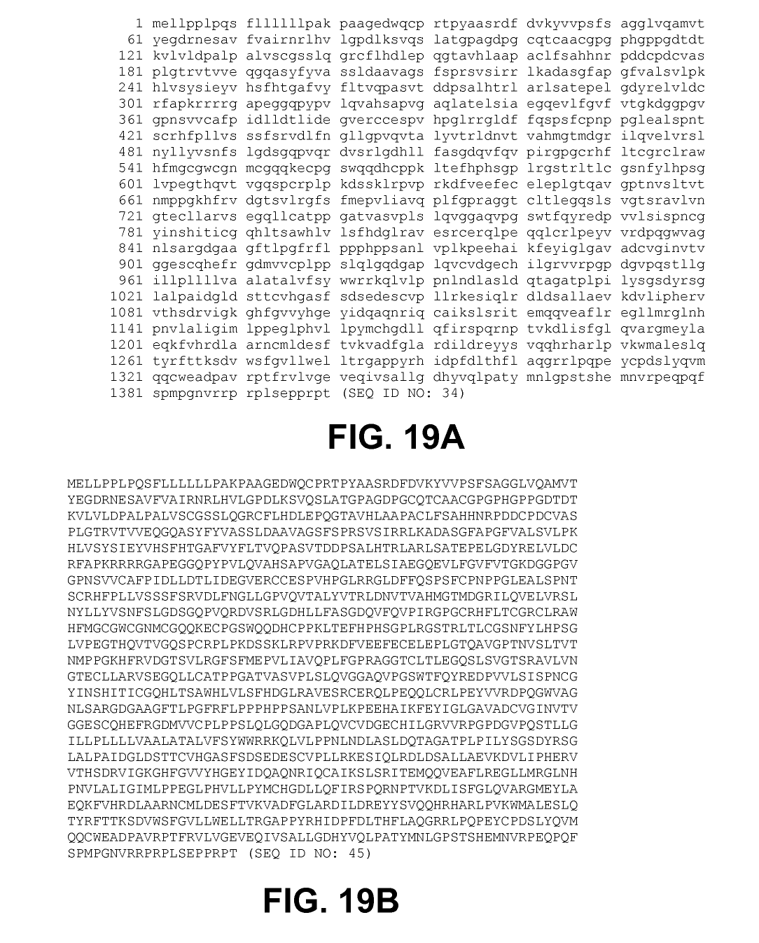

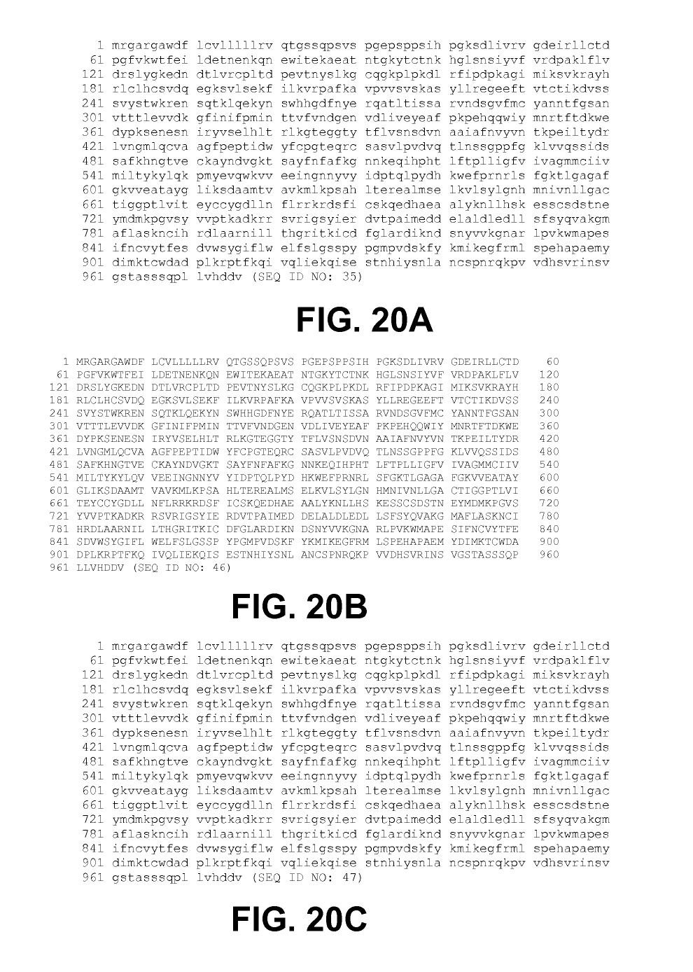

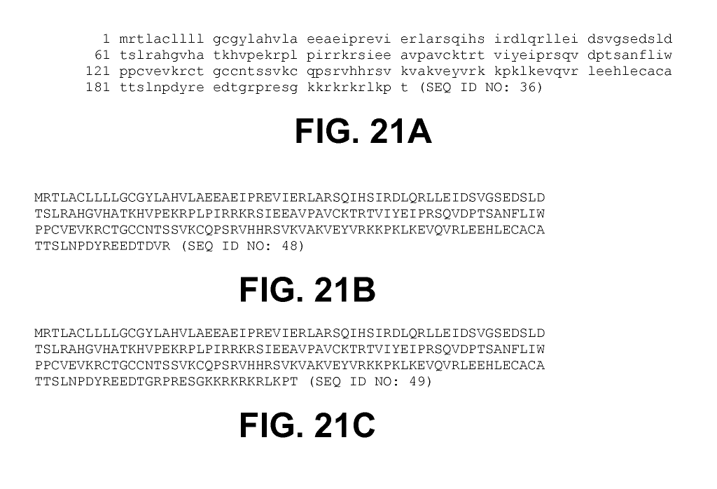

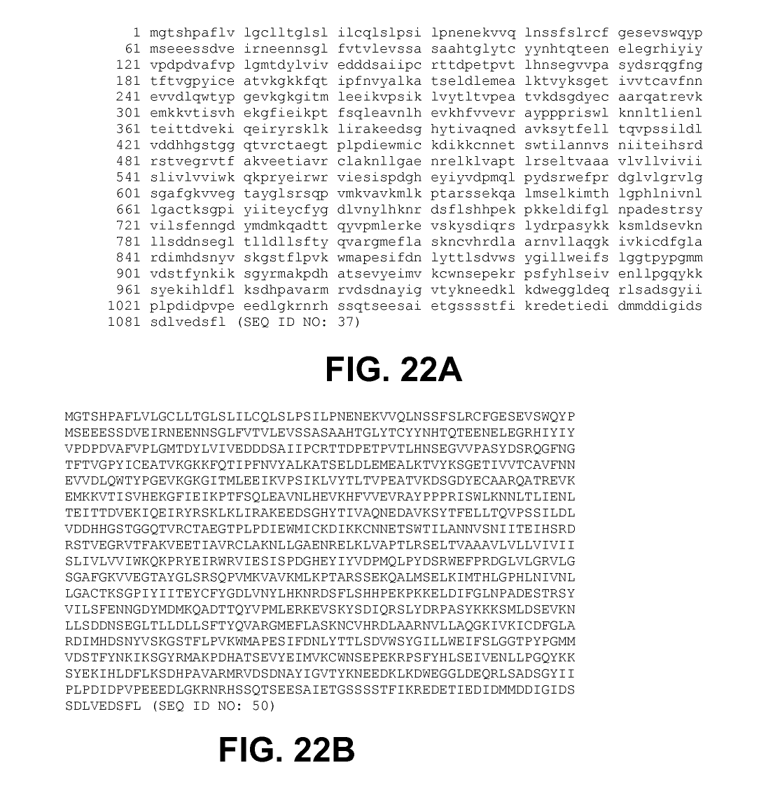

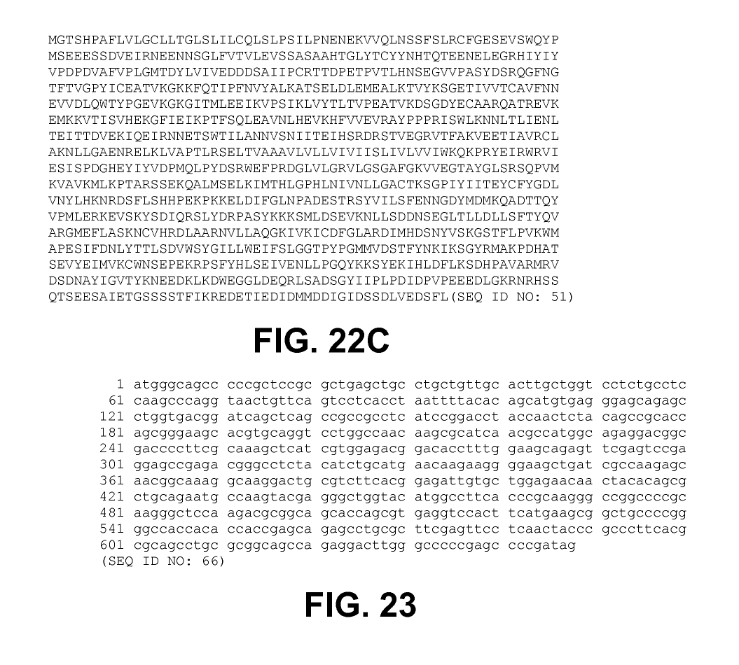

Other experiments include testing the immunological effects of the Ab-1 from ex vivo studies and measure T-cell mediated cytotoxicity in monoclonal antibody treated tumor cells. In addition, dual targeted strategy using antibody and FGFR selective tyrosine kinase inhibitors (TKIs) in combination, e.g. R04383596 or Pazopanib (as illustrated in FIG. 15), can be analyzed. Particularly, Ab-1 effects on TKI drug resistant tumor cells are investigated. This dual targeted strategy has shown in EGFR-targeted cancers enhanced anti-tumor activity (Huang et al., (2004) Cancer Res. 64: 5355-5362).

### Example 6

### FGFR2-IIIc as a Potential Biomarker for Circulating Tumor Cells in Prostate Cancer

This example examines the presence of FGFR2IIIc receptors on cell lines resembling hormone refractory prostate cancer in peripheral blood cells from patients via testing positive for CTC by the conventional, approved histopathology methods. Additional verification of the tumor nature of cells positive for FGFR2IIIc expression can be done by PCR methods using isoform specific primer sets. The outcome of this study is to recognize a subgroup of patients, whose tumor and metastasis is dependent on the expression of FGFR2 isoform Inc, and an additional enhancement of the specificity of the existing and approved CTC test using Ep-CAM.

Specifically, this example evaluates the feasibility to detect and enrich CTCs (or epithelial-to-mesenchymal (ETM) transformed prostate tumor cells) expressing the oncogenic receptor FGFR2 isoform IIIc (FGFR2IIIc) with an isoform specific antibody. The initial focus is the identification and detection of circulating cells bearing FGFR-2IIIc from peripheral blood from patients with known metastatic diseases. Once the positive detection and specificity data are established, the technical optimization can be pursued on sensitivity of the detection in a healthy control group, patients with benign prostate hyperplasia, and prostate cancer patients. The following are the specific aims for this example:
1) Investigate the presence of FGFR2-IIIc positive CTCs from peripheral blood and confirm these cells as cancer cells.
2) Enrich and isolate FGFR2-IIIc positive CTCs by immunomagnetic purification.
3) Confirm the existence of CTC-bearing FGFR2-IIIc receptor by RT-PCR analysis using exon-specific PCR primers.

This example is a feasibility study for the utility of FGFR2IIIc as a valid biomarker for identification of prostate cancer CTCs for diagnosing metastatic disease and malignancy in asymptomatic prostate cancer patients. Further study focuses on:
a. Optimize the detection method by quantitative recovery of spiked-in prostate tumor cells (FGFR2-IIIc positive, such as DU145, PC3) in peripheral blood samples.
b. Enumerate FGFR2IIIc positive cells in peripheral blood from patients before and after prostatectomy, before and after TURP (transurethral prostate resection) for benign prostate.
c. Collect large data sets from asymptomatic and symptomatic hormone-refractory prostate cancer patients to determine the diagnostic and prognostic value of the test.

### Example 6.1

### Significance of the Test for Detection of Circulating Tumor Cells

Metastatic tumor cells spread through the blood or lymph as “circulating tumor cells” (CTCs), and bone marrow as “disseminated tumor cells” (DTCs). CTCs and DTCs represent unique diagnostic and therapeutic targets. Circulating tumor cells are extremely rare in patients with nonmalignant diseases but are present in various metastatic carcinomas with a wide range of frequencies (Allard et al. (2004) Clin Cancer Res. 10:6897-6904). Some clinical studies indicate the assessment of CTCs can assist physicians in monitoring and predicting cancer progression and in evaluating response to therapy in patients with metastatic cancer (Berrepoot et al., (2004) Ann Oncol. 15:139-145; Aquino et al., (2002) J. Chemother. 14:412-416; Katoh et al., (2004) Anticancer Res. 24:1421-1425). Recent studies on relationship between post-treatment CTC count and overall survival (OS) in castration-resistant prostate cancer (CRPC) indicated that CTC counts predicted OS better than PSA decrement algorithms at all time points (de Bono et al., (2008) Clin Cancer Res. 4(19):6302-9).

Current CTC detection methods based on epithelial markers, e.g. Ep-CAM may miss FGFR2-IIIc positive circulating tumor cells, because FGFR2 IIc expression on prostate cancer cells is associated with loss of epithelial markers and gain of mesenchymal markers (Moffa and Ethier (2007) J Cell Physiol. 210(3):720-31).

Several frequently used methodologies for detecting CTCs used either alone or in combination can be categorized as—
i) Molecular biological: e.g. RT-PCR (reverse-transcription PCR)
ii) Immunochemical: e.g. antibody-coupled magnetic beads; immunofluorescent microscopy; flow cytometry (FACS) analysis

RT-PCR offers a highly sensitive method to detect genes. However, PCR detects living cells, dead cells, and free DNA, resulting in potential false-positives. The specificity of the amplified target genes is a limiting factor for its diagnostic or prognostic value.

Tumor cells bearing an oncogenic receptor FGFR2-IIIc isoform found on androgen-independent tumors are believed to be responsible for invasive tumor growth and metastasis by intra-organ spread and by dissemination via blood stream, respectively. The identification of prostate-derived circulating tumor cells (CTCs) by a FGFR2-IIIc specific antibody is an alternative step in the diagnosis and staging of prostate cancer. The continued presence of these cells in the circulation after prostatectomy may indicate the development of metastatic disease. Therefore, CTC detection shown in this example can provide additional sensitivity and specificity for diagnosing metastasis in HPCR patients.

### Example 6.2

### CTC Enumeration for Overall Survival Prediction in Prostate Cancer

It has been known that CTC enumeration at baseline and over time by immunomagnetic capture more reliably predicts unfavorable outcome measured as overall survival than PSA levels and changes (de Bono et al., (2008) Clin Cancer Res. 14(19):6302-9; Danila et al., (2007) Clin Cancer Res. 13(23):7053-8). The detection of FGFR2-IIIc as a potential biomarker for CTCs in prostate cancer adds an additional level of understanding to the molecular mechanisms of prostate cancer and metastatic disease. In addition, this assay can provide a specific test for currently unrecognized HRPC subpopulation.

### Example 6.3

### Immunomagnetic Purification of DU145 Tumor Cells Spiked in Normal Blood

To purify DU145 tumor cells spiked in normal blood, the following protocol can be used.
a. Prepare immunomagnetic beads: monoclonal antibody against FGFR2-IIIc (in 0.1 mg/ml in PBS containing 1% BSA) is immobilized onto magnetic beads pre-coupled with goat anti-mouse Fc (from Becton Dickinson) by an overnight incubation at 4° C.
b. Tumor cell spiking experiment: PC12 (FGFR2-IIIc negative), DU145 (FGFR2-IIIc positive) tumor cells are preload with fluorescent dye calcein AM for viable cells (from Molecular Probes, Eugene, Oreg.) by a 5-minute incubation at 37° C. Labeled cells are spiked in 7.5 ml normal blood cells at the following ratios: 1000 cells, 500 cells, 100 cells, 50 cells, 10 cells. These labeled cells are exposed to immunomagnetic beads, recovered fluorescent cells can be counted under a fluorescent microscope using a 20× magnification or by flow cytometry FACS analysis. A constant recovery rate is the demonstration of good efficiency of immunomagnetic selection of FGFR2-IIIc positive cells.

### Example 6.4

### Detection and Enrichment of CTCs from Patients with Prostate Cancer

This follows published procedure and the instrumentation by Veridex (de Bono et al., (2008) Clin Cancer Res. 14(19):6302-9). Blood samples from patients can be used to detect CTCs bearing FGFR2-IIIc by previously reported procedure (Berrepoot et al., (2004) Ann Oncol. 15:139-145; Aquino et al., (2002) J. Chemother. 14:412-416; Katoh et al. (2004) Anticancer Res. 24:1421-1425; Allard et al. (2004) Clin Cancer Res. 10:6897-6904; de Bono et al., (2008) Clin Cancer Res. 14(19):6302-9). Essentially, blood samples are drawn into 10-ml EDTA Vacutainer tubes (Becton Dickinson) to which a cell preservative was added (Berrepoot et al., (2004) Ann Oncol. 15:139-145; Aquino et al., (2002) J. Chemother. 14:412-416; Katoh et al. (2004) Anticancer Res. 24:1421-1425; Allard et al. (2004) Clin Cancer Res. 10:6897-6904; de Bono et al., (2008) Clin Cancer Res. 14(19):6302-9). Samples are maintained at room temperature and processed within 72 hours after collection. Cells are allowed to incubate with anti-FGFR2-IIIc loaded magnetic beads. Fluorescent nucleic acid dye DAPI (4,2-diamidino-2-phenylindole dihydrochloride) is used to stain nucleated cells. The identification and enumeration of FGFR2-IIIc positive CTCs can be performed with the use of the CellSpotter Analyzer, a semiautomated fluorescence-based microscopy system that permits computer-generated reconstruction of cellular images. Circulating tumor cells are counted as nucleated cells expressing FGFR2-IIIc. To confirm the epithelial cell nature of the isolated CTCs, cells can be double stained with an epithelial cell marker cytokeratin19, labeled with another fluorescent dye phycoerytherin (PE).

### Example 6.5

### RT-PCR of FGFR2-IIIc isoform

To demonstrate the specificity of the immunomagnetic selection for FGFR2-IIIc positive CTCs, RT-PCR experiment can be performed to confirm the isoform FGFR2-IIIc expression in isolated cells. RNA can be isolated using RNeasy Mini Kit, including RNase-Free DNase Set (Qiagen, Hilden, Germany). For reverse transcription, RNA is diluted in 15 μl of RNase-free water, incubated for 5 min at 65° C., and placed on ice. A 7.5 μl mixture containing 2 μl of oligo-p(dT)15 primer (0.8 μg/μl), 2 μl of deoxynucleoside triphosphate (5 mM), 0.5 μl of RNAsin (40 units/μl), 1 μl of Omniscript Reverse Transcriptase (4.5 units/μl), and 2 μl of reverse transcriptase buffer (×10) are prepared and added to the diluted RNA. After incubation at 37° C. for 1 h, Omniscript Reverse Transcriptase is inactivated for 5 min at 95° C., and cDNA can be stored at −20° C. PCR amplification is performed using IIIc exon specific primers (IIIc-F: aggttctcaaggccgccggtgt (SEQ ID NO: 71) and IIIc-R: caaccatgcagagtgaaagga (SEQ ID NO: 72). IIIb exon specific primers (IIIb-F: ggttctcaagcactcgggga (SEQ ID NO: 69) and IIIb-R: gccaggcagactggttggcc (SEQ ID NO: 70)) are used as reference. The design of isoform-specific primers for PCR analysis is shown in FIG. 16. Tumor cells, PC-3 (IIIb positive) and DU145 (IIIc positive) can be used as positive controls for the PCR experiments. The PCR product should be appear as a 140 base-pair band on agarose gel.

### Example 6.6

### Other Experiments

Other experiments include optimizing the test protocol and validate/enhance the clinical relevance of enumeration of CTCs in HRPC patient's clinical outcomes. Biostatistical methods are used for data analysis and interrogation.

### Example 7

### FGFR2 III-c as a Biomarker for Detection of Hormone-Refractory Prostate Cancer

This example describes the establishment of an immunohistochemical staining (1HC) test for the detection of invasive, hormone-resistant prostate cancer. As disclosed, FGFR2 isoform IIIc, is associated with androgen-independent tumor growth and metastasis, and it is expressed on the surface of cancerous prostate tissue.

Several biomarkers have been developed as immunohistochemical (IHC) staining tests for prostate cancer. These include prostate-specific antigen (PSA), prostate-specific membrane antigen (PSMA), prostate stem cell antigen (PSCA), androgen receptor (AR), chromogranin, synaptophysin, MIB-1, and a-methylacyl-CoA racemase (AMACR). These markers are not specific for metastatic status or metastatic potential. An examination of FGFR2 isoform IIIc and IIIb expression in the biopsies and surgical specimens should provide additional information for patient with hormone refractory disease and a potential for metastasis.

Prostate cancer cell lines DU145 and LNCaP, were originally obtained from the American Type Culture Collection (ATCC). These cell cultures are maintained using standard protocols.

Prostate cancer with matched normal prostate tissue Arrays, and human tissue arrays can be obtained from US Biomax (Rockville, Md. 20849); Multi-Tumor Microarrays will be obtained from Invitrogen (CA). These tissues should be in compliant with FDA and regulatory requirements. Patients' clinical data, e.g. Gleason score and pathological stage of disease are available. Patient's private information is protected.

IHC tests can be used for surgical samples from radical prostatectomy, or needle biopsies (NBX) and transurethral resections of the prostate (TURP).

Objectives of this example include (i) establishing the IHC test protocol by using tumor cell lines fixed in paraffin as cell pellets; (ii) evaluating the utility of this IHC diagnostic test using tissue specimens from patients with prostate cancer and patients with benign prostate hypertrophy.

The following experiments can be conducted:
i. Investigate the differential expression of the two functionally distinct isoform receptors of FGFR2 in prostate tumor cell lines, DU145 (IIIc positive) and LNCaP (IIIb positive). Demonstrate the specificities and sensitivity of mAbs to each isoform for IHC application. Establish the IHC protocol.
ii. Stain 30-organ tissue arrays to survey the distinct tissue distribution of IIIb and IIIc using Tissue Arrays from US Biomax (Rockville, Md. 20849)
iii. Examine about 20 cases of each, prostate carcinomas, benign prostate hyperplasias (BPHs), to distinguish between neoplastic and noncancerous tissues (Tumor arrays from Invitrogen, CA)
iv. Analyze IHC staining and define the grading and staining patterns; e.g. positive, negative scoring, and FGFR2-IIIc expression patterns in tumor tissues/cells (work with a pathologist expert)
v. Explore the clinical relevance of biomarker expression with disease severity and evaluate the benefit of using targeted antibody drug for blocking metastatic disease.

This biomarker can also be used in combination with other IHC tissue markers in a multi-biomarker analysis.

### Example 7.1

### IHC staining Protocol

A general staining protocol for mAbs against FGFR2 receptor is described below. Experimental conditions can be optimized for each mAbs of anti-FGFR-IIIc or IIIb.

Immunohistochemistry with Paraffin-Embedded Tissue Sections

Antibodies: Monoclonal anti-FGFR2IIIc and anti-FGFR2IIIb antibodies are generated as described above. A monoclonal anti-Cytokeratin (Pan) Clone AE1/AE3 antibody is from Zymed (San Francisco, Calif.) and a polyclonal anti-PSA antibody is from Dako Cytomation. Secondary antibody coupled with peroxidase, ChemMate™, DAKO Envision™ Detection Kit are from Dako Cytomation (Denmark).

Diaminobenzidine (DAB) can be used as chromogen followed by Meyer's hematoxylin counterstaining.

### I. Preparation of Slides

Cell pellets are created from DU145 and LNCaP cells, fixed in 10% formalin overnight, and then processed in the regular manner for pathology specimens to produce paraffin embedded cell blocks. Tissue slides are already prepared from paraffin-blocks by commercial vendors.

### II. Deparaffinization

1. Label all slides clearly with a pencil, noting antibody and dilution.
2. Deparaffinize and rehydrate as follows: Three times for 5 minutes in xylene; two times for 5 minutes in 100% ethanol; two times for 5 minutes in 95% ethanol: and once for 5 minutes in 80% ethanol,
3. Place all sections in endogenous blocking solution (methanol 2% hydrogen peroxide) for 20 minutes at room temperature.
4. Rinse sections twice for 5 minutes each in deionized water.
5. Rinse sections twice for 5 minutes in phosphate buffered saline (PBS), pH 7.4.

### III. Blocking and Staining

1. Block all sections with PBS/1% bovine serum albumin (PBA) for 1 hour at room temperature.
2. Incubate sections in rabbit serum diluted in PBA (2%) for 30 minutes at room temperature to reduce non-specific binding of antibody. Perform the incubation in a sealed humidity chamber to prevent air-drying of the tissue sections.
3. Gently shake off excess antibody and cover sections with mAb diluted in PBA. Replace the lid of the humidity chamber and incubate either at room temperature for 1 hour or overnight at 4° C.
4. Rinse sections twice for 5 minutes in PBS, shaking gently.
5. Gently remove excess PBS and cover sections with diluted HRP conjugated rabbit anti-mouse antibody in PBA for 30 minutes to 1 hour at room temperature in the humidity chamber.
6. Rinse sections twice for 5 minutes in PBS, shaking gently.

### Scoring IHC Staining:

Stained slides can be evaluated by experienced urological pathologists (consultants). A scoring method will be developed based on a varying degree of staining intensity and percentage of cells staining. The evaluation will be done in a blinded fashion.

### Statistical Analysis:

Univariate associations between FGFR2 expression and Gleason score, clinical stage and progression to androgen-independence can be calculated using Fisher's Exact Test. For all analyses, p<0.05 was considered statistically significant.

### Example 7.3

### Other Experiments

Other experiments include the utility of this assay in the selection and characterization of patients in the clinical development of AB-1 as a therapeutic agent in prostate cancer. Retrospective analysis of larger data sets from patients and correlation analyses on biomarker expression profile with disease severity and clinico-pathological parameters are also conducted.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. An isolated isoform-specific inhibitor which inhibits one or more oncogenic isoform-associated activities, and/or specifically binds to an oncogenic isoform polypeptide or nucleic acid, wherein the isoform-specific inhibitor is selected from the group consisting of an antibody molecule, a soluble receptor polypeptide, a receptor fusion, a peptide or a peptide analog thereof and an inhibitor nucleic acid, and wherein the oncogenic isoform polypeptide or nucleic acid is an oncogenic isoform of FGFR2, FGFR2, FGFR1, RON receptor tyrosine kinase, KIT receptor tyros kinase, PDGF and PDGF-receptor alpha.

2. (canceled)

3. (canceled)

4. The isoform-specific inhibitor of claim 1, wherein the oncogenic isoform polypeptide or nucleic acid is selected from the group con of FGFR2 isoform IIIc or a fragment thereof; isoform FGFR1L having a deletion of about 105 amino acids between exons 7 and 8; isoform RONΔ160 having an in-frame deletion of about 109 amino acids skipping exons 5 and 6 of the extracellular domain of RON; a KIT isoform having deletion of exon 11; exon 6-deleted PDGF isoform or a fragment thereof, and a PDGFR-alpha isoform having an in-frame deletion of exons 7 and 8.

5.-7. (canceled)

8. The isoform-specific inhibitor of claim 1, wherein the antibody molecule binds specifically to at least one epitope located in the alternative spliced form of Exon III from about amino acids 301 to 360 of FGFR2-IIIc (SEQ ID NO:2); about amino acids 314 to 324 of FGFR2-IIIc (AAGVNTTDKEI, SEQ ID NO:4); about amino acids 328 to 337 of FGFR2-IIIc (YIRNVTFEDA, SEQ ID NO:6); about amino acids 350 to 353 of FGFR2-IIIc (ISFH, SEQ ID NO:8), or an amino acid sequence encoded by a nucleotide sequence of SEQ ID NOs: 1, 3, 5 or 7; or an amino acid or nucleotide sequence substantially identical thereto.

9. The isoform-specific inhibitor of claim 1, wherein the antibody molecule shows less than 10% cross-reactivity with an amino acid sequence of human FGFR2 isoform IIIb selected from the group consisting of about amino acids 314 to 351 of human FGFR2 isoform IIIb (HSGINSSNAEVLALFNVTEADAGEYICKVSNYIGQANQ; SEQ ID NO: 56); about amino acids 314 to 328 of human FGFR2 isoform IIIb (HSGINSSNAEVLALF; SEQ ID NO: 57); and about amino acids 340 to 351 of human FGFR2 isoform IIIb (CKVSNYIGQANQ; SEQ ID NO: 58).

10-19. (canceled)

20. The isoform-specific inhibitor of claim 9, wherein the antibody molecule is a human, humanized, chimeric, camelid or in vitro generated antibody, or an antigen-binding fragment thereof chosen from one or more of: an Fab, F(ab′)₂, Fv, a single chain Fv fragment, a single domain antibody, a diabody (dAb), a bivalent or bispecific antibody or fragment thereof, a single domain variant thereof, or a camelid antibody.

21-27. (canceled)

28. The isoform-specific inhibitor of claim 1, which is functionally linked to one or more other molecular entities chosen from one or more of an antibody, a toxin, a radioisotope, a cytotoxic or cytostatic agent, and a label.

29.-33. (canceled)

34. A pharmaceutical composition comprising the isoform-specific inhibitor of claim 1 and a pharmaceutically acceptable carrier, excipient or stabilizer.

35. (canceled)

36. An isolated nucleic acid encoding the isoform-specific inhibitor of claim 1.

37. A host cell comprising the nucleic acid of claim 8.

38. A method of producing the isoform-specific inhibitor of claim 1, comprising culturing a host cell comprising a nucleic acid encoding the isoform-specific inhibitor under conditions suitable for gene expression.

39. A method of providing an isoform-binding antibody molecule that specifically binds to an oncogenic isoform polypeptide, comprising:
providing a isoform-specific epitope up to 60 amino acids in length or less;
obtaining an antibody molecule that specifically binds to the isoform polypeptide; and
evaluating one of more of:
(a) evaluating if the antibody molecule specifically binds to the isoform polypeptide;
(b) evaluating if there is a decrease in binding between the antibody molecule and the isoform polypeptide in the presence of one or more isoform-specific epitope, or
(c) evaluating efficacy of the antibody molecule in modulating inhibiting the activity of the oncogenic isoform polypeptide,
wherein the isoform-specific epitope comprises an amino acid sequence selected from the group consisting of:
(i) an amino acid sequence identical to the alternative spliced form of Exon III from about amino acids 301 to 360 of FGFR2-IIIc (SEQ ID NO:2); about amino acids 314 to 324 of FGFR2-IIIc (AAGVNTTDKEI, SEQ ID NO:4); about amino acids 328 to 337 of FGFR2-IIIc (YIRNVTFEDA, SEQ ID NO:6); about amino acids 350 to 353 of FGFR2-IIIc (ISFH, SEQ ID NO:8), or an amino acid sequence encoded by a nucleotide sequence of SEQ ID NOs: 1, 3, 5 or 7; or an amino acid or nucleotide sequence substantially identical thereto;
(ii) an amino acid sequence identical the junctional region between Ig-II and Ig-III of FGFR1L (SEQ ID NO:10) or a fragment thereof, or an amino acid sequence encoded by a nucleotide sequence of SEQ ID NO:9 or a fragment thereof; or an amino acid or nucleotide sequence substantially identical thereto;
(iii) an amino acid sequence identical to the junctional region between exon 4 and exon 7 of isoform RONΔ160 (SEQ ID NO:12) or a fragment thereof, or an amino acid sequence encoded by a nucleotide sequence of SEQ ID NO:11 or a fragment thereof; or an amino acid or nucleotide sequence substantially identical thereto;
(iv) an amino acid sequence identical to the junctional region of KIT between exons 10 and 12 of SEQ ID NO:14 or a fragment thereof, or an amino acid sequence encoded by a nucleotide sequence of SEQ ID NO:13 or a fragment thereof; or an amino acid or nucleotide sequence substantially identical thereto;
(v) an amino acid sequence identical to the junctional region of PDGF between exons 5 and 7 of SEQ ID NO:16 or a fragment thereof, or an amino acid sequence encoded by a nucleotide sequence of SEQ ID NO:15 or a fragment thereof; or an amino acid or nucleotide sequence substantially identical thereto; and
(vi) an amino acid sequence identical to the junctional region of PDGFR-alpha between exons 6 and 9 of SEQ ID NO:18 or a fragment thereof, or an amino acid sequence encoded by a nucleotide sequence of SEQ ID NO:17 or a fragment thereof; or an amino acid or nucleotide sequence substantially identical thereto.

40. An isoform-specific epitope comprising an amino acid sequence up to 60 amino acids in length or less selected from the group consisting of:
(i) an amino acid sequence identical to the alternative spliced form of Exon III from about amino acids 301 to 360 of FGFR2-IIIc (SEQ ID NO:2); about amino acids 314 to 324 of FGFR2-IIIc (AAGVNTTDKEI, SEQ ID NO:4); about amino acids 328 to 337 of FGFR2-IIIc (YIRNVTFEDA, SEQ ID NO:6); about amino acids 350 to 353 of FGFR2-IIIc (ISFH, SEQ ID NO:8), or an amino acid sequence encoded by a nucleotide sequence of SEQ ID NOs: 1, 3, 5 or 7; or an amino acid or nucleotide sequence substantially identical thereto;
(ii) an amino acid sequence identical the junctional region between Ig-II and Ig-III of FGFR1L (SEQ ID NO:10) or a fragment thereof, or an amino acid sequence encoded by a nucleotide sequence of SEQ ID NO:9 or a fragment thereof; or an amino acid or nucleotide sequence substantially identical thereto;
(iii) an amino acid sequence identical to the junctional region between exon 4 and exon 7 of isoform RONΔ160 (SEQ ID NO:12) or a fragment thereof, or an amino acid sequence encoded by a nucleotide sequence of SEQ ID NO:11 or a fragment thereof; or an amino acid or nucleotide sequence substantially identical thereto;
(iv) an amino acid sequence identical to the junctional region of KIT between exons 10 and 12 of SEQ ID NO:14 or a fragment thereof, or an amino acid sequence encoded by a nucleotide sequence of SEQ ID NO:13 or a fragment thereof; or an amino acid or nucleotide sequence substantially identical thereto;
(v) an amino acid sequence identical to the junctional region of PDGF between exons 5 and 7 of SEQ ID NO:16 or a fragment thereof, or an amino acid sequence encoded by a nucleotide sequence of SEQ ID NO:15 or a fragment thereof; or an amino acid or nucleotide sequence substantially identical thereto; and
(vi) an amino acid sequence identical to the junctional region of PDGFR-alpha between exons 6 and 9 of SEQ ID NO:18 or a fragment thereof, or an amino acid sequence encoded by a nucleotide sequence of SEQ ID NO:17 or a fragment thereof; or an amino acid or nucleotide sequence substantially identical thereto.

41. A method of reducing cell growth or proliferation, or inducing the killing of a cancerous or tumor cell expressing an oncogenic isoform of FGFR2, FGFR1, RON receptor tyrosine kinase, KIT receptor tyrosine kinase, PDGF and PDGF-receptor alpha, comprising: contacting the cancerous or tumor cell, or a cell proximal to the cancerous or tumor cell, with one or more isoform-specific inhibitors of claim 9, in an amount sufficient to reduce the expression or activity of the oncogenic isoform, thereby reducing the cell growth or proliferation of, or inducing the killing of, the cancerous or tumor cell.

42. (canceled)

43. (canceled)

44. A method treating or preventing a primary, recurring or metastasizing cancer of the prostate, testis, breast, pancreas, bladder, gastrointestinal, lung squamous cell carcinoma, non-small cell lung carcinoma, tyroid cancer, endometrial carcinoma, hematopoietic cancers, or brain, comprising administering to a subject the isoform-specific inhibitor of claim 9, in an amount effective to treat or prevent such cancer.

45. (canceled)

46. The method of claim 44, wherein the cancer is hormone-resistant or refractory prostate cancer associated with elevated expression of FGFR2-IIIc.

47.-49. (canceled)

50. The method of claim 46, wherein the subject has abnormal levels of one or more markers for a cancer selected from the group consisting of prostate-specific antigen (PSA), prostate specific membrane antigen (PSMA), prostate stem cell antigen (PSCA), androgen receptor (AR), chromogranin, synaptophysin, MIB-1, and α-methylacyl-CoA racemase (AMACR).

51.-54. (canceled)

55. A method for detecting the presence of an oncogenic isoform polypeptide or gene expression product in a sample in vitro, comprising: (i) contacting the sample (and optionally, a reference sample) with the isoform-specific inhibitor of claim 1, under conditions that allow interaction of the isoform binding molecule and the polypeptide or gene expression product to occur, and (ii) detecting formation of a complex between the isoform binding molecule, and the sample (and optionally, the reference sample).

56. (canceled)

57. (canceled)

58. A method for detecting the presence of an oncogenic isoform polypeptide or gene expression product in vivo, comprising: (i) administering to a subject the isoform-specific inhibitor of claim 1, under conditions that allow interaction of the isoform binding molecule and the polypeptide or gene expression product to occur; and (ii) detecting formation of a complex between the isoform binding molecule and the polypeptide or gene expression product.

59. A method of monitoring treatment or progression of, diagnosing and/or staging a cancerous disorder, in a subject, comprising: (i) identifying a subject having, or at risk of having, the cancerous disorder, (ii) obtaining a sample of a tissue or cell affected with the cancerous disorder, (iii) contacting said sample or a control sample with the isoform-specific inhibitor of claim 1, under conditions that allow an interaction of the binding molecule and the isoform polypeptide or gene product to occur, and (iv) detecting formation of a complex, wherein an increase in the formation of the complex between the isoform-binding molecule with respect to a reference sample is indicative of the cancerous disorder or the stage of the cancerous disorder.

60-64. (canceled)

65. The method of claim 59, further comprising the step of monitoring the subject for a change in one or more of: tumor size; level or expression of FGFR2IIIc; level of circulating prostate-derived FGFR2IIIc-expressing cells; level or expression of one or more of epithelial cell markers (Ep-CAM). FGF8, stromal derived factor α (SDF α), VEGF121, mesenchymal markers, PSA, PSMA, PSCA, AR, chromogranin, synaptophysin, MIB-1, AMACR, alkaline phosphatase, or serum hemoglobin; the rate of appearance of new lesions; the appearance of new disease-related symptoms; or the size of soft tissue mass.


SEQUENCE LISTING (81 sequences)

SEQ ID NO: 1 - DNA, 180 bp, Homo sapiens
tacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctg

SEQ ID NO: 2 - protein, 60 aa, Homo sapiens
YGPDGLPYLKVLKAAGVNTTDKEIEVLYIRNVTFEDAGEYTCLAGNSIGISFHSAWLTVL

SEQ ID NO: 3 - DNA, 33 bp, Homo sapiens
  CDS (1)..(33)
gccgccggtgttaacaccacggacaaagagatt

SEQ ID NO: 4 - protein, 11 aa, Homo sapiens
AAGVNTTDKEI

SEQ ID NO: 5 - DNA, 30 bp, Homo sapiens
  CDS (1)..(30)
tatattcggaatgtaacttttgaggacgct

SEQ ID NO: 6 - protein, 10 aa, Homo sapiens
YIRNVTFEDA

SEQ ID NO: 7 - DNA, 12 bp, Homo sapiens
  CDS (1)..(12)
atatcctttcac

SEQ ID NO: 8 - protein, 4 aa, Homo sapiens
ISFH

SEQ ID NO: 9 - DNA, 90 bp, Homo sapiens
  CDS (1)..(90)
aatggcaaagaattcaaacctgaccacagaattggaggctacaagactgctggagttaataccaccgacaaagagatggaggtgcttcac

SEQ ID NO: 10 - protein, 30 aa, Homo sapiens
NGKEFKPDHRIGGYKTAGVNTTDKEMEVLH

SEQ ID NO: 11 - DNA, 90 bp, Homo sapiens
  CDS (1)..(90)
cctggctcctggcaacaggaccactgcccacctaagcttactgaggagccagtgctgatagcagtgcaacccctctttggcccacgggca

SEQ ID NO: 12 - protein, 30 aa, Homo sapiens
PGSWQQDHCPPKLTEEPVLIAVQPLFGPRA

SEQ ID NO: 13 - DNA, 90 bp, Homo sapiens
  CDS (1)..(90)
atgatgtgcattattgtgatgattctgacctacaaatatttacaggttgttgaggagataaatggaaacaattatgtttacatagaccca

SEQ ID NO: 14 - protein, 30 aa, Homo sapiens
MMCIIVMILTYKYLQVVEEINGNNYVYIDP

SEQ ID NO: 15 - DNA, 54 bp, Homo sapiens
  CDS (1)..(54)
tgcgcgaccacaagcctgaatccggattatcgggaagaggacacggatgtgagg

SEQ ID NO: 16 - protein, 18 aa, Homo sapiens
CATTSLNPDYREEDTDVR

SEQ ID NO: 17 - DNA, 90 bp, Homo sapiens
  CDS (1)..(90)
ctcactgagatcaccactgatgtggaaaagattcaggaaataaggaataatgaaacttcctggactattttggccaacaatgtctcaaac

SEQ ID NO: 18 - protein, 30 aa, Homo sapiens
LTEITTDVEKIQEIRNNETSWTILANNVSN

SEQ ID NO: 19 - protein, 821 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 20 - DNA, 2466 bp, Homo sapiens
atggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcaccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtggtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgaggaatacttggacctcagccaacctctcgaacagtattcacctagttaccctgacacaagaagttcttgttcttcaggagatgattctgttttttctccagaccccatgccttacgaaccatgccttcctcagtatccacacataaacggcagtgttaaaacatga

SEQ ID NO: 21 - protein, 822 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 22 - protein, 682 aa, Homo sapiens
MVSWGRFICLVVVTMATLSLARPSFSLVEDTTLEPEDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRIPPNPSLMSIFRK

SEQ ID NO: 23 - protein, 817 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNERYKLLPCPDKHNKRCKPEERGDLTEAGAAGSSRCVDSRKRVRQEKISTG

SEQ ID NO: 24 - protein, 819 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNERILTLTTNENFQSTSGREGTEIHALQCLRSEVTPAISCESPLADTGSKVPN

SEQ ID NO: 25 - protein, 819 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNESFQSSLKSSSTGIPGWPPGSEVFSEVAFRGILNYDIERPILCAGSKKIYDI

SEQ ID NO: 26 - protein, 830 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEGRLPAWASQEKENSQTSLFAISHVTLSSISKTRSSAKRDEKPGSSPHLALVRSQGLPQSVVP

SEQ ID NO: 27 - protein, 771 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEPLS

SEQ ID NO: 28 - protein, 768 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNE

SEQ ID NO: 29 - protein, 254 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVGSQGL

SEQ ID NO: 30 - protein, 705 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 31 - protein, 769 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEI

SEQ ID NO: 32 - protein, 821 aa, Homo sapiens; identical to SEQ ID NO: 19

SEQ ID NO: 33 - protein, 822 aa, Homo sapiens [sequence omitted]

SEQ ID NO: 34 - protein, 1400 aa, Homo sapiens
MELLPPLPQSFLLLLLLPAKPAAGEDWQCPRTPYAASRDFDVKYVVPSFSAGGLVQAMVTYEGDRNESAVFVAIRNRLHVLGPDLKSVQSLATGPAGDPGCQTCAACGPGPHGPPGDTDTKVLVLDPALPALVSCGSSLQGRCFLHDLEPQGTAVHLAAPACLFSAHHNRPDDCPDCVASPLGTRVTVVEQGQASYFYVASSLDAAVAGSFSPRSVSIRRLKADASGFAPGFVALSVLPKHLVSYSIEYVHSFHTGAFVYFLTVQPASVTDDPSALHTRLARLSATEPELGDYRELVLDCRFAPKRRRRGAPEGGQPYPVLQVAHSAPVGAQLATELSIAEGQEVLFGVFVTGKDGGPGVGPNSVVCAFPIDLLDTLIDEGVERCCESPVHPGLRRGLDFFQSPSFCPNPPGLEALSPNTSCRHFPLLVSSSFSRVDLFNGLLGPVQVTALYVTRLDNVTVAHMGTMDGRILQVELVRSLNYLLYVSNFSLGDSGQPVQRDVSRLGDHLLFASGDQVFQVPIRGPGCRHFLTCGRCLRAWHFMGCGWCGNMCGQQKECPGSWQQDHCPPKLTEFHPHSGPLRGSTRLTLCGSNFYLHPSGLVPEGTHQVTVGQSPCRPLPKDSSKLRPVPRKDFVEEFECELEPLGTQAVGPTNVSLTVTNMPPGKHFRVDGTSVLRGFSFMEPVLIAVQPLFGPRAGGTCLTLEGQSLSVGTSRAVLVNGTECLLARVSEGQLLCATPPGATVASVPLSLQVGGAQVPGSWTFQYREDPVVLSISPNCGYINSHITICGQHLTSAWHLVLSFHDGLRAVESRCERQLPEQQLCRLPEYVVRDPQGWVAGNLSARGDGAAGFTLPGFRFLPPPHPPSANLVPLKPEEHAIKFEYIGLGAVADCVGINVTVGGESCQHEFRGDMVVCPLPPSLQLGQDGAPLQVCVDGECHILGRVVRPGPDGVPQSTLLGILLPLLLLVAALATALVFSYWWRRKQLVLPPNLNDLASLDQTAGATPLPILYSGSDYRSGLALPAIDGLDSTTCVHGASFSDSEDESCVPLLRKESIQLRDLDSALLAEVKDVLIPHERVVTHSDRVIGKGHFGVVYHGEYIDQAQNRIQCAIKSLSRITEMQQVEAFLREGLLMRGLNHPNVLALIGIMLPPEGLPHVLLPYMCHGDLLQFIRSPQRNPTVKDLISFGLQVARGMEYLAEQKFVHRDLAARNCMLDESFTVKVADFGLARDILDREYYSVQQHRHARLPVKWMALESLQTYRFTTKSDVWSFGVLLWELLTRGAPPYRHIDPFDLTHFLAQGRRLPQPEYCPDSLYQVMQQCWEADPAVRPTFRVLVGEVEQIVSALLGDHYVQLPATYMNLGPSTSHEMNVRPEQPQFSPMPGNVRRPRPLSEPPRPT

SEQ ID NO: 35 - protein, 976 aa, Homo sapiens [sequence omitted]

SEQ ID NO: 36 - protein, 211 aa, Homo sapiens [sequence omitted]

SEQ ID NO: 37 - protein, 1089 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 38 - protein, 822 aa, Homo sapiens [sequence omitted]

SEQ ID NO: 39 - protein, 820 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 40 - protein, 820 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 41 - protein, 150 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSACPDLQEAKWCSASFHSITPLPFGLGTRLSD

SEQ ID NO: 42 - protein, 302 aa, Homo sapiens
MWSWKCLLFWAVLVTATLCTARPSPTLPEQDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKVIMAPVFVGQSTGKETTVSGAQVPVGRLSCPRMGSFLTLQAHTLHLSRDLATSPRTSNRGHKVEVSWEQRAAGMGGAGL

SEQ ID NO: 43 - protein, 61 aa, Homo sapiens
MWSWKCLLFWAVLVTATLCTARPSPTLPEQACPDLQEAKSCSASFHSITPLPFGLGTRLSD

SEQ ID NO: 44 - protein, 300 aa, Homo sapiens
MWSWKCLLFWAVLVTATLCTARPSPTLPEQDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKVIMAPVFVGQSTGKETTVSGAQVPVGRLSCPRMGSFLTLQAHTLHLSRDLATSPRTSNRGHKVEVSWEQRAAGMGGAGL

SEQ ID NO: 45 - protein, 1400 aa, Homo sapiens; identical to SEQ ID NO: 34

SEQ ID NO: 46 - protein, 967 aa, Homo sapiens
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTPLLIGFVIVAGMMCIIVMILTYKYLQVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 47 - protein, 976 aa, Homo sapiens [sequence omitted]

SEQ ID NO: 48 - protein, 196 aa, Homo sapiens
MRTLACLLLLGCGYLAHVLAEEAEIPREVIERLARSQIHSIRDLQRLLEIDSVGSEDSLDTSLRAHGVHATKHVPEKRPLPIRRKRSIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTDVR

SEQ ID NO: 49 - protein, 211 aa, Homo sapiens [sequence omitted]

SEQ ID NO: 50 - protein, 1089 aa, Homo sapiens; identical to SEQ ID NO: 37

SEQ ID NO: 51 - protein, 1007 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 52 - protein, 820 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 53 - protein, 366 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQGRRC

SEQ ID NO: 54 - DNA, 1476 bp, Homo sapiens
atggtcagctggggtcgtttcatctgcctggtcgtggtcaccatggcaaccttgtccctggcccggccctccttcagtttagttgaggataccacattagagccagaaggagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtggtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagagatctgacaaaactcacacatgcccaccgtgcccagcacctgaactcctggggggaccgtcagtcttcctcttccccccaaaacccaaggacaccctctacatcacccgggaacctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgaagttccactacacgcagaagagcctctccctgtctccgggtaaatga

SEQ ID NO: 55 - protein, 491 aa, Homo sapiens
MVSWGRFICLVVVTMATLSLARPSFSLVEDTTLEPEGAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLERSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 56 - protein, 38 aa, Homo sapiens
HSGINSSNAEVLALFNVTEADAGEYICKVSNYIGQANQ

SEQ ID NO: 57 - protein, 15 aa, Homo sapiens
HSGINSSNAEVLALF

SEQ ID NO: 58 - protein, 12 aa, Homo sapiens
CKVSNYIGQANQ

SEQ ID NO: 59 - protein, 10 aa, Artificial Sequence
  Description of Artificial Sequence Synthetic peptide
GGGGSGGGGS

SEQ ID NO: 60 - DNA, 114 bp, Homo sapiens
cactcggggataaatagttccaatgcagaagtgctggctctgttcaatgtgaccgaggcggatgctggggaatatatatgtaaggtctccaattatatagggcaggccaaccag

SEQ ID NO: 61 - DNA, 45 bp, Homo sapiens
cactcggggataaatagttccaatgcagaagtgctggctctgttc

SEQ ID NO: 62 - DNA, 36 bp, Homo sapiens
tgtaaggtctccaattatatagggcaggccaaccag

SEQ ID NO: 63 - DNA, 2469 bp, Homo sapiens
atggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaagcactcggggataaatagttccaatgcagaagtgctggctctgttcaatgtgaccgaggcggatgctggggaatatatatgtaaggtctccaattatatagggcaggccaaccagtctgcctggctcactgtcctgccaaaacagcaagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgaggaatacttggacctcagccaacctctcgaacagtattcacctagttaccctgacacaagaagttcttgttcttcaggagatgattctgttttttctccagaccccatgccttacgaaccatgccttcctcagtatccacacataaacggcagtgttaaaacatga

SEQ ID NO: 64 - DNA, 174 bp, Homo sapiens
tacgggcccgacgggctgccctacctcaaggttctcaagcactcggggataaatagttccaatgcagaagtgctggctctgttcaatgtgaccgaggcggatgctggggaatatatatgtaaggtctccaattatatagggcaggccaaccagtctgcctggctcactgtcctg

SEQ ID NO: 65 - protein, 58 aa, Homo sapiens
YGPDGLPYLKVLKHSGINSSNAEVLALFNVTEADAGEYICKVSNYIGQANQSAWLTVL

SEQ ID NO: 66 - DNA, 648 bp, Homo sapiens
atgggcagcccccgctccgcgctgagctgcctgctgttgcacttgctggtcctctgcctccaagcccaggtaactgttcagtcctcacctaattttacacagcatgtgagggagcagagcctggtgacggatcagctcagccgccgcctcatccggacctaccaactctacagccgcaccagcgggaagcacgtgcaggtcctggccaacaagcgcatcaacgccatggcagaggacggcgaccccttcgcaaagctcatcgtggagacggacacctttggaagcagagttcgagtccgaggagccgagacgggcctctacatctgcatgaacaagaaggggaagctgatcgccaagagcaacggcaaaggcaaggactgcgtcttcacggagattgtgctggagaacaactacacagcgctgcagaatgccaagtacgagggctggtacatggccttcacccgcaagggccggccccgcaagggctccaagacgcggcagcaccagcgtgaggtccacttcatgaagcggctgccccggggccaccacaccaccgagcagagcctgcgcttcgagttcctcaactacccgcccttcacgcgcagcctgcgcggcagccagaggacttgggcccccgagccccgatag

SEQ ID NO: 67 - protein, 48 aa, Rattus sp.
LPYLKVLKAAGVNTTDKEIEVLYIRNVTFEDAGEYTCLAGNSIGISFH

SEQ ID NO: 68 - protein, 46 aa, Rattus sp.
LPYLKVLKHSGINSSNAEVLALFNVTEMDAGEYICKVSNYIGQANQ

SEQ ID NO: 69 - DNA, 20 bp, Artificial Sequence
  Description of Artificial Sequence Synthetic primer
ggttctcaagcactcgggga

SEQ ID NO: 70 - DNA, 20 bp, Artificial Sequence
  Description of Artificial Sequence Synthetic primer
gccaggcagactggttggcc

SEQ ID NO: 71 - DNA, 22 bp, Artificial Sequence
  Description of Artificial Sequence Synthetic primer
aggttctcaaggccgccggtgt

SEQ ID NO: 72 - DNA, 21 bp, Artificial Sequence
  Description of Artificial Sequence Synthetic primer
caaccatgcagagtgaaagga

SEQ ID NO: 73 - protein, 5 aa, Artificial Sequence
  Description of Artificial Sequence Synthetic peptide
SGGGG

SEQ ID NO: 74 - protein, 10 aa, Artificial Sequence
  Description of Artificial Sequence Synthetic peptide
SGGGGSGGGG

SEQ ID NO: 75 - protein, 15 aa, Artificial Sequence
  Description of Artificial Sequence Synthetic peptide
SGGGGSGGGGSGGGG

SEQ ID NO: 76 - protein, 20 aa, Artificial Sequence
  Description of Artificial Sequence Synthetic peptide
SGGGGSGGGGSGGGGSGGGG

SEQ ID NO: 77 - protein, 25 aa, Artificial Sequence
  Description of Artificial Sequence Synthetic peptide
SGGGGSGGGGSGGGGSGGGGSGGGG

SEQ ID NO: 78 - protein, 30 aa, Artificial Sequence
  Description of Artificial Sequence Synthetic peptide
SGGGGSGGGGSGGGGSGGGGSGGGGSGGGG

SEQ ID NO: 79 - protein, 35 aa, Artificial Sequence
  Description of Artificial Sequence Synthetic peptide
SGGGGSGGGGSGGGGSGGGGSGGGGSGGGGSGGGG

SEQ ID NO: 80 - protein, 40 aa, Artificial Sequence
  Description of Artificial Sequence Synthetic peptide
SGGGGSGGGGSGGGGSGGGGSGGGGSGGGGSGGGGSGGGG

SEQ ID NO: 81 - protein, 6 aa, Artificial Sequence
  Description of Artificial Sequence Synthetic 6xHis tag
HHHHHH